SEQUENCE LISTING (131 sequences)

SEQ ID NO: 1 - DNA, 24 bp, ARTIFICIAL SEQUENCE
  FORWARD PRIMER FOR RSV-A
agatcaacttctgtcatccagcaa

SEQ ID NO: 2 - DNA, 25 bp, ARTIFICIAL SEQUENCE
  REVERSE PRIMER FOR RSV-A
gcacatcataattaggagtatcaat

SEQ ID NO: 3 - DNA, 26 bp, ARTIFICIAL SEQUENCE
  FORWARD PRIMER FOR RSV-B
aagatgcaaatcataaattcacagga

SEQ ID NO: 4 - DNA, 33 bp, ARTIFICIAL SEQUENCE
  REVERSE PRIMER FOR RSV-B
tgatatccagcatctttaagtatctttatagtg

SEQ ID NO: 5 - DNA, 25 bp, ARTIFICIAL SEQUENCE
  FORWARD PRIMER FOR INFLUENZA A  (N1)
atggtaatggtgtttggataggaag

SEQ ID NO: 6 - DNA, 22 bp, ARTIFICIAL SEQUENCE
  REVERSE PRIMER FOR INFLUENZA A (N1)
aatgctgctcccactagtccag

SEQ ID NO: 7 - DNA, 21 bp, ARTIFICIAL SEQUENCE
  FORWARD PRIMER FOR INFLUENZA A (N2)
aagcatggctgcatgtttgtg

SEQ ID NO: 8 - DNA, 24 bp, ARTIFICIAL SEQUENCE
  REVERSE PRIMER FOR INFLUENZA A (N2)
accaggatatcgaggataacagga

SEQ ID NO: 9 - DNA, 24 bp, ARTIFICIAL SEQUENCE
  FORWARD PRIMER FOR INFLUENZA A (M)
catggaatggctaaagacaagacc

SEQ ID NO: 10 - DNA, 24 bp, ARTIFICIAL SEQUENCE
  REVERSE PRIMER FOR INFLUENZA A (M)
aagtgcaccagcagaataactgag

SEQ ID NO: 11 - DNA, 23 bp, ARTIFICIAL SEQUENCE
  FORWARD PRIMER FOR INFLUENZA (H1)
ggtgttcatcacccgtctaacat

SEQ ID NO: 12 - DNA, 23 bp, ARTIFICIAL SEQUENCE
  REVERSE PRIMER FOR INFLUENZA A (H1)
gtgtttgacacttcgcgtcacat

SEQ ID NO: 13 - DNA, 27 bp, ARTIFICIAL SEQUENCE
  FORWARD PRIMER FOR INFLUENZA A (H2)
gctatgcaaactaaacggaatycctcc

SEQ ID NO: 14 - DNA, 26 bp, ARTIFICIAL SEQUENCE
  REVERSE PRIMER FOR INFLUENZA A (H2)
tattgttgtacgatcctttggcaacc

SEQ ID NO: 15 - DNA, 23 bp, ARTIFICIAL SEQUENCE
  FORWARD PRIMER FOR INFLUENZA (H3)
gctactgagctggttcagagttc

SEQ ID NO: 16 - DNA, 24 bp, ARTIFICIAL SEQUENCE
  REVERSE PRIMER FOR INFLUENZA A (H3)
gaagtcttcattgataaactccag

SEQ ID NO: 17 - DNA, 24 bp, ARTIFICIAL SEQUENCE
  FORWARD PRIMER FOR INFLUENZA (H5)
ttactgttacacatgcccaagaca

SEQ ID NO: 18 - DNA, 24 bp, ARTIFICIAL SEQUENCE
  REVERSE PRIMER FOR INFLUENZA (H5)
aggyttcactccatttagatcgca

SEQ ID NO: 19 - DNA, 22 bp, ARTIFICIAL SEQUENCE
  FORWARD PRIMER FOR INFLUENZA B
agaccagagggaaactatgccc

SEQ ID NO: 20 - DNA, 24 bp, ARTIFICIAL SEQUENCE
  REVERSE PRIMER INFLUENZA B
ctgtcgtgcattataggaaagcac

SEQ ID NO: 21 - DNA, 20 bp, ARTIFICIAL SEQUENCE
  FORWARD PRIMER FOR SARS CoV
aagcctcgccaaaaacgtac

SEQ ID NO: 22 - DNA, 20 bp, ARTIFICIAL SEQUENCE
  REVERSE PRIMER FOR SARS CoV
aagtcagccatgttcccgaa

SEQ ID NO: 23 - DNA, 20 bp, ARTIFICIAL SEQUENCE
  FORWARD PRIMER FOR 229E CoV
ggcgcaagaattcagaacca

SEQ ID NO: 24 - DNA, 19 bp, ARTIFICIAL SEQUENCE
  REVERSE PRIMER TO 229E CoV
taagagccgcagcaactgc

SEQ ID NO: 25 - DNA, 21 bp, ARTIFICIAL SEQUENCE
  FORWARD PRIMER FOR OC43 CoV
tgtgcctattgcaccaggagt

SEQ ID NO: 26 - DNA, 19 bp, ARTIFICIAL SEQUENCE
  REVERSE PRIMER FOR OC43 CoV
cccgatcgacaatgtcagc

SEQ ID NO: 27 - DNA, 24 bp, ARTIFICIAL SEQUENCE
  FORWARD PRIMER FOR METAPNEUMOVIRUS EUROPEAN
aaccgtgtactaagtgatgcactc

SEQ ID NO: 28 - DNA, 22 bp, ARTIFICIAL SEQUENCE
  REVERSE PRIMER FOR METAPNEUMOVIRUS EUROPEAN
cattgtttgaccggccccataa

SEQ ID NO: 29 - DNA, 24 bp, ARTIFICIAL SEQUENCE
  FORWARD PRIMER FOR METAPNEUMOVIRUS CANADIAN
aagtccaaaggcaggrctgttatc

SEQ ID NO: 30 - DNA, 25 bp, ARTIFICIAL SEQUENCE
  REVERSE PRIMER FOR METAPNEUMOVIRUS CANADIAN
cctgaagcattrccaagaacaacac

SEQ ID NO: 31 - DNA, 27 bp, ARTIFICIAL SEQUENCE
  FORWARD PRIMER FOR PARAINFLUENZA 1
tacttttgacacatttagttccaggag

SEQ ID NO: 32 - DNA, 27 bp, ARTIFICIAL SEQUENCE
  REVERSE PRIMER FOR PARAINFLUENZA 1
cggtacttctttgaccaggtataattg

SEQ ID NO: 33 - DNA, 21 bp, ARTIFICIAL SEQUENCE
  FORWARD PRIMER FOR PARAINFLUENZA 2
ggacttggaacaagatggcct

SEQ ID NO: 34 - DNA, 25 bp, ARTIFICIAL SEQUENCE
  REVERSE PRIMER FOR PARAINFLUENZA 2
agcatgagagcytttaatttctgga

SEQ ID NO: 35 - DNA, 24 bp, ARTIFICIAL SEQUENCE
  FORWARD PRIMER FOR PARAINFLUENZA 3
gctttcagacaagatggaacagtg

SEQ ID NO: 36 - DNA, 23 bp, ARTIFICIAL SEQUENCE
  REVERSE PRIMER FOR PARAINFLUENZA 3
gcatkattgacccaatctgatcc

SEQ ID NO: 37 - DNA, 24 bp, ARTIFICIAL SEQUENCE
  FORWARD PRIMER FOR PARAINFLUENZA 4A
aacagaaggaaatgatggtggaac

SEQ ID NO: 38 - DNA, 20 bp, ARTIFICIAL SEQUENCE
  REVERSE PRIMER FOR PARAINFLUENZA 4A
tgctgtggatgtatgggcag

SEQ ID NO: 39 - DNA, 25 bp, ARTIFICIAL SEQUENCE
  FORWARD PRIMER FOR PARAINFLUENZA 4B
agaagaaaacaacgatgagacaagg

SEQ ID NO: 40 - DNA, 23 bp, ARTIFICIAL SEQUENCE
  REVERSE PRIMER FOR PARAINFLUENZA 4B
gtttccctggttcactctcttca

SEQ ID NO: 41 - DNA, 25 bp, ARTIFICIAL SEQUENCE
  FORWARD PRIMER FOR CYTOMEGALOVIRUS
tacagcacgctcaacaccaacgcct

SEQ ID NO: 42 - DNA, 25 bp, ARTIFICIAL SEQUENCE
  REVERSE PRIMER FOR CYTOMEGALOVIRUS
cccggccttcaccaccaaccgaaaa

SEQ ID NO: 43 - DNA, 25 bp, ARTIFICIAL SEQUENCE
  FORWARD PRIMER FOR MEASLES VIRUS
caagcatcatgatygccattcctgg

SEQ ID NO: 44 - DNA, 26 bp, ARTIFICIAL SEQUENCE
  REVERSE PRIMER FOR MEASLES VIRUS
cctgaatcyctgcctatgatgggttt

SEQ ID NO: 45 - DNA, 18 bp, ARTIFICIAL SEQUENCE
  FORWARD PRIMER FOR ADENOVIRUS
cccmttyaaccaccaccg

SEQ ID NO: 46 - DNA, 20 bp, ARTIFICIAL SEQUENCE
  REVERSE PRIMER FOR ADENOVIRUS
acatccttbckgaagttcca

SEQ ID NO: 47 - DNA, 28 bp, ARTIFICIAL SEQUENCE
  FORWARD PRIMER FOR ENTEROVIRUS
tcctccggcccctgaatgcggctaatcc

SEQ ID NO: 48 - DNA, 25 bp, ARTIFICIAL SEQUENCE
  REVERSE PRIMER FOR ENTEROVIRUS
gaaacacggwcacccaaagtastcg

SEQ ID NO: 49 - DNA, 21 bp, ARTIFICIAL SEQUENCE
  FORWARD PRIMER FOR M. PNEUMONIAE
ccaaccaaacaacaacgttca

SEQ ID NO: 50 - DNA, 20 bp, ARTIFICIAL SEQUENCE
  REVERSE PRIMER FOR M. PNEUMONIAE
accttgactggaggccgtta

SEQ ID NO: 51 - DNA, 23 bp, ARTIFICIAL SEQUENCE
  FORWARD PRIMER FOR L. PNEUMOPHILAE
gcatwgatgttartccggaagca

SEQ ID NO: 52 - DNA, 20 bp, ARTIFICIAL SEQUENCE
  REVERSE PRIMER FOR L. PNEUMOPHILAE
cggttaaagccaattgagcg

SEQ ID NO: 53 - DNA, 20 bp, ARTIFICIAL SEQUENCE
  FORWARD PRIMER FOR C. PNEUMONIAE
catggtgtcattcgccaagt

SEQ ID NO: 54 - DNA, 20 bp, ARTIFICIAL SEQUENCE
  REVERSE PRIMER FOR C. PNEUMONIAE
cgtgtcgtccagccatttta

SEQ ID NO: 55 - DNA, 21 bp, ARTIFICIAL SEQUENCE
  FORWARD PRIMER FOR RIFT VALLEY FEVER VIRUS
ggattgacctgtgcctgttgc

SEQ ID NO: 56 - DNA, 26 bp, ARTIFICIAL SEQUENCE
  REVERSE PRIMER FOR RIFT VALLEY FEVER VIRUS
gcattagaaatgtcctcttttgctgc

SEQ ID NO: 57 - DNA, 23 bp, ARTIFICIAL SEQUENCE
  FORWARD PRIMER FOR CCHF
agaacacgtgccgcttacgccca

SEQ ID NO: 58 - DNA, 27 bp, ARTIFICIAL SEQUENCE
  REVERSE PRIMER FOR CCHF
ccattcyttyttraactcytcaaacca

SEQ ID NO: 59 - DNA, 27 bp, ARTIFICIAL SEQUENCE
  FORWARD PRIMER FOR EBOLA VIRUS
aacaccgggtcttaattcttatatcaa

SEQ ID NO: 60 - DNA, 28 bp, ARTIFICIAL SEQUENCE
  REVERSE PRIMER FOR EBOLA VIRUS
ggtggtaaaattcccatagtagttcttt

SEQ ID NO: 61 - DNA, 20 bp, ARTIFICIAL SEQUENCE
  FORWARD PRIMER FOR MARBURG VIRUS
ttccgtcacaagccgaaatt

SEQ ID NO: 62 - DNA, 29 bp, ARTIFICIAL SEQUENCE
  REVERSE PRIMER FOR MARBURG VIRUS
ttattttagttgagaaaagaggttcatgc

SEQ ID NO: 63 - DNA, 18 bp, ARTIFICIAL SEQUENCE
  FORWARD PRIMER FOR WEST NILE VIRUS
gctccgctgtccctgtga

SEQ ID NO: 64 - DNA, 21 bp, ARTIFICIAL SEQUENCE
  REVERSE PRIMER FOR WEST NILE VIRUS
cactctcctcctgcatggatg

SEQ ID NO: 65 - DNA, 22 bp, ARTIFICIAL SEQUENCE
  FORWARD PRIMER FOR ST. LOUIS ENCEPHALITIS VIRUS
catttgttcagctgtcccagtc

SEQ ID NO: 66 - DNA, 22 bp, ARTIFICIAL SEQUENCE
  REVERSE PRIMER FOR ST. LOUIS ENCEPHALITIS VIRUS
ctcacccttcccatgaattgac

SEQ ID NO: 67 - DNA, 24 bp, ARTIFICIAL SEQUENCE
  FORWARD PRIMER FOR HERPES SIMPLEX VIRUS
cccggatgcggtccagacgattat

SEQ ID NO: 68 - DNA, 24 bp, ARTIFICIAL SEQUENCE
  REVERSE PRIMER FOR HERPES SIMPLEX VIRUS
cccgcggaggttgtacaaaaagct

SEQ ID NO: 69 - DNA, 25 bp, ARTIFICIAL SEQUENCE
  FORWARD PRIMER FOR HIV-1
ttcttggagcagcggaagcacatgg

SEQ ID NO: 70 - DNA, 25 bp, ARTIFICIAL SEQUENCE
  REVERSE PRIMER FOR HIV-1
ttmatgccccagacgtagttcaaca

SEQ ID NO: 71 - DNA, 19 bp, ARTIFICIAL SEQUENCE
  FORWARD PRIMER FOR HIV-2
ggctgcacgccctatgata

SEQ ID NO: 72 - DNA, 19 bp, ARTIFICIAL SEQUENCE
  REVERSE PRIMER FOR HIV-2
tctgcatggctgcttgatg

SEQ ID NO: 73 - DNA, 18 bp, ARTIFICIAL SEQUENCE
  FORWARD PRIMER FOR N. MENIGITIDIS
tctgaagccattggccgt

SEQ ID NO: 74 - DNA, 18 bp, ARTIFICIAL SEQUENCE
  REVERSE PRIMER FOR N. MENIGITIDIS
caaacacaccacgcgcat

SEQ ID NO: 75 - DNA, 22 bp, ARTIFICIAL SEQUENCE
  FORWARD PRIMER FOR S. PNEUMONIAE
agcgatagctttctccaagtgg

SEQ ID NO: 76 - DNA, 23 bp, ARTIFICIAL SEQUENCE
  REVERSE SEQUENCE FOR S. PNEUMONIAE
cttagccaacaaatcgtttaccg

SEQ ID NO: 77 - DNA, 27 bp, ARTIFICIAL SEQUENCE
  FORWARD PRIMER FOR H. INFLUENZAE
aagctccttgmattttttgtattagaa

SEQ ID NO: 78 - DNA, 23 bp, ARTIFICIAL SEQUENCE
  REVERSE PRIMER FOR H. INFLUENZAE
gctgaattggcttrgataccgag

SEQ ID NO: 79 - DNA, 22 bp, ARTIFICIAL SEQUENCE
  FORWARD PRIMER FOR INFLUENZA B
agaccagagggaaactatgccc

SEQ ID NO: 80 - DNA, 24 bp, ARTIFICIAL SEQUENCE
  REVERSE PRIMER FOR INFLUENZA B
ctgtcgtgcattataggaaagcac

SEQ ID NO: 81 - DNA, 20 bp, ARTIFICIAL SEQUENCE
  FORWARD PRIMER DIRECTED TO SARS CoV
aagcctcgccaaaaacgtac

SEQ ID NO: 82 - DNA, 20 bp, ARTIFICIAL SEQUENCE
  REVERSE PRIMER DIRECTED TO SARS CoV
aagtcagccatgttcccgaa

SEQ ID NO: 83 - DNA, 20 bp, ARTIFICIAL SEQUENCE
  FORWARD PRIMER DIRECTED TO 229E-CoV
ggcgcaagaattcagaacca

SEQ ID NO: 84 - DNA, 19 bp, ARTIFICIAL SEQUENCE
  REVERSE PRIMER DIRECTED TO 229E-CoV
taagagccgcagcaactgc

SEQ ID NO: 85 - DNA, 21 bp, ARTIFICIAL SEQUENCE
  FORWARD PRIMER FOR OC43 CoV
tgtgcctattgcaccaggagt

SEQ ID NO: 86 - DNA, 19 bp, ARTIFICIAL SEQUENCE
  REVERSE PRIMER FOR OC43 CoV
cccgatcgacaatgtcagc

SEQ ID NO: 87 - DNA, 25 bp, ARTIFICIAL SEQUENCE
  FORWARD PRIMER FOR CYTOMEGALOVIRUS
tacagcacgctcaacaccaacgcct

SEQ ID NO: 88 - DNA, 25 bp, ARTIFICIAL SEQUENCE
  REVERSE PRIMER DIRECTED TO CYTOMEGALOVIRUS
cccggccttcaccaccaaccgaaaa

SEQ ID NO: 89 - DNA, 23 bp, ARTIFICIAL SEQUENCE
  FORWARD PRIMER FOR VARICELLA ZOSTER VIRUS
acgtggatcgtcggatcagttgt

SEQ ID NO: 90 - DNA, 23 bp, ARTIFICIAL SEQUENCE
  REVERSE PRIMER FOR VARICELLA ZOSTER VIRUS
tcgctatgtgctaaaacacgcgg

SEQ ID NO: 91 - DNA, 25 bp, ARTIFICIAL SEQUENCE
  FORWARD PRIMER FOR MEASLES VIRUS
caagcatcatgatygccattcctgg

SEQ ID NO: 92 - DNA, 26 bp, ARTIFICIAL SEQUENCE
  REVERSE PRIMER FOR MEASLES VIRUS
cctgaatcyctgcctatgatgggttt

SEQ ID NO: 93 - DNA, 18 bp, ARTIFICIAL SEQUENCE
  REVERSE PRIMER FOR ADENOVIRUS
cccmttyaaccaccaccg

SEQ ID NO: 94 - DNA, 20 bp, ARTIFICIAL SEQUENCE
  REVERSE PRIMER FOR ADNEOVIRUS
acatccttbckgaagttcca

SEQ ID NO: 95 - DNA, 28 bp, ARTIFICIAL SEQUENCE
  FORWARD PRIMER FOR ENTEROVIRUS
tcctccggcccctgaatgcggctaatcc

SEQ ID NO: 96 - DNA, 25 bp, ARTIFICIAL SEQUENCE
  REVERSE PRIMER FOR ENTEROVIRUS
gaaacacggwcacccaaagtastcg

SEQ ID NO: 97 - DNA, 19 bp, ARTIFICIAL SEQUENCE
  Primer directed to SARS virus
acgtcgtttaaaccgtagt

SEQ ID NO: 98 - DNA, 25 bp, ARTIFICIAL SEQUENCE
  Forward Primer for Enterovirus A/B 702/495
tccggcccctgaatgcggctaatcc

SEQ ID NO: 99 - DNA, 20 bp, ARTIFICIAL SEQUENCE
  Forward Primer for Enterovirus A/B 702/495
cccctgaatgcggctaatcc

SEQ ID NO: 100 - DNA, 22 bp, ARTIFICIAL SEQUENCE
  Foward primer for S. Pneumoniae
agcgatagctttctccaagtgg

SEQ ID NO: 101 - DNA, 23 bp, ARTIFICIAL SEQUENCE
  Reverse primer for S. Pneumoniae
cttagccaacaaatcgtttaccg

SEQ ID NO: 102 - DNA, 21 bp, ARTIFICIAL SEQUENCE
  FORWARD PRIMER RSV A  VIRUS
ggtgcagggcaagtgatgtta

SEQ ID NO: 103 - DNA, 21 bp, ARTIFICIAL SEQUENCE
  REVERSE PRIMER RSV A  VIRUS
gccagcagcattgcctaatac

SEQ ID NO: 104 - DNA, 21 bp, ARTIFICIAL SEQUENCE
  FORWARD PRIMER RSV A  VIRUS
caggaacaagcccaattatca

SEQ ID NO: 105 - DNA, 20 bp, ARTIFICIAL SEQUENCE
  reverse PRIMER RSV A  VIRUS
ctcttaaaccatggcatctc

SEQ ID NO: 106 - DNA, 22 bp, ARTIFICIAL SEQUENCE
  FORWARD PRIMER RSV B  VIRUS
atggttcagggcaagtaatgct

SEQ ID NO: 107 - DNA, 21 bp, ARTIFICIAL SEQUENCE
  REVERSE PRIMER RSV A  VIRUS
tctcctcccaacttctgtgca

SEQ ID NO: 108 - DNA, 22 bp, ARTIFICIAL SEQUENCE
  FORWARD PRIMER RSV B  VIRUS
tctggcaccaacatcatcaatc

SEQ ID NO: 109 - DNA, 23 bp, ARTIFICIAL SEQUENCE
  REVERSE PRIMER RSV B  VIRUS
ggggtgagatcttctttgaagct

SEQ ID NO: 110 - DNA, 21 bp, ARTIFICIAL SEQUENCE
  PRIMER MYCOPLASMA 1
ccaaccaaacaacaacgttca

SEQ ID NO: 111 - DNA, 20 bp, ARTIFICIAL SEQUENCE
  PRIMER DIRECTED TO MYCOPLASMA 1
accttgactggaggccgtta

SEQ ID NO: 112 - DNA, 24 bp, ARTIFICIAL SEQUENCE
  PRIMER MYCOPLASMA 2
ccgcgaagagcaatgaaaaactcc

SEQ ID NO: 113 - DNA, 24 bp, ARTIFICIAL SEQUENCE
  PRIMER FOR MYCOPLASMA 2
tcgaggcggatcatttggggaggt

SEQ ID NO: 114 - DNA, 23 bp, ARTIFICIAL SEQUENCE
  primer for legionella 1
gcatwgatgttartccggaagca

SEQ ID NO: 115 - DNA, 20 bp, ARTIFICIAL SEQUENCE
  PRIMER FOR LEGIONELLA 1
cggttaaagccaattgagcg

SEQ ID NO: 116 - DNA, 21 bp, ARTIFICIAL SEQUENCE
  PRIMER FOR LEGIONELLA 2
aaaggcatgcaagacgctatg

SEQ ID NO: 117 - DNA, 25 bp, ARTIFICIAL SEQUENCE
  PRIMER FOR LEGIONELLA 2
tgttaagaacgtctttcatttgctg

SEQ ID NO: 118 - DNA, 21 bp, ARTIFICIAL SEQUENCE
  PRIMER FOR LEGIONELLA 3
ggcgactatagcgatttggaa

SEQ ID NO: 119 - DNA, 22 bp, ARTIFICIAL SEQUENCE
  PRIMER FOR LEGIONELLA 3
gcgatgacctactttcgcatga

SEQ ID NO: 120 - DNA, 20 bp, ARTIFICIAL SEQUENCE
  PRIMER FOR CHLAMYDIA
catggtgtcattcgccaagt

SEQ ID NO: 121 - DNA, 20 bp, ARTIFICIAL SEQUENCE
  PRIMER FOR CHLAMYDIA
cgtgtcgtccagccatttta

SEQ ID NO: 122 - DNA, 23 bp, ARTIFICIAL SEQUENCE
  PRIMER FOR HA1
ggtgttcatcacccgtctaacat

SEQ ID NO: 123 - DNA, 23 bp, ARTIFICIAL SEQUENCE
  PRIMER FOR HA1
gtgtttgacacttcgcgtcacat

SEQ ID NO: 124 - DNA, 28 bp, ARTIFICIAL SEQUENCE
  PRIMER FOR HA2
gctatgcaaaactaaacggaatycctcc

SEQ ID NO: 125 - DNA, 26 bp, ARTIFICIAL SEQUENCE
  PRIMER FOR HA2
tattgttgtacgatcctttggcaacc

SEQ ID NO: 126 - DNA, 23 bp, ARTIFICIAL SEQUENCE
  PRIMER FOR HA3
gctactgagctggttcagagttc

SEQ ID NO: 127 - DNA, 24 bp, ARTIFICIAL SEQUENCE
  PRIMER FOR HA3
gaagtcttcattgataaactccag

SEQ ID NO: 128 - DNA, 23 bp, ARTIFICIAL SEQUENCE
  PRIMER FOR HA3
gctactgagctggttcagagttc

SEQ ID NO: 129 - DNA, 20 bp, ARTIFICIAL SEQUENCE
  PRIMER FOR HA3
atgctgagcgactccagtcc

SEQ ID NO: 130 - DNA, 24 bp, ARTIFICIAL SEQUENCE
  PRIMER FOR HA5-HUMAN
ttactgttacacatgcccaagaca

SEQ ID NO: 131 - DNA, 24 bp, ARTIFICIAL SEQUENCE
  PRIMER FOR HA5-HUMAN
aggyttcactccatttagatcgca

(19) United States
(12) Patent Application Publication
(10) Pub. No.: US 2006/0003352 A1
(43) Pub. Date: Jan. 5, 2006

(54) Mass tag PCR for mutliplex diagnostics

(76) Inventors: W. Ian Lipkin, New York, NY (US); Jingyue Ju, Englewood Cliffs, NJ (US); Thomas Briese, White Plains, NY (US)

Correspondence Address: COOPER & DUNHAM, LLP, 1185 AVENUE OF THE AMERICAS, NEW YORK, NY, 10036, US

(21) Appl. No.: 11/119,231

(22) Filed: Apr. 28, 2005

Related U.S. Application Data
(60) Provisional application No. 60/566,967, filed on Apr. 29, 2004.

(51) Int. Cl. C12Q 1/68; C12P 19/34

(52) U.S. Cl. 435/6; 435/91.2

(57) ABSTRACT

This invention provides a mass tag-based method for simultaneously detecting in a sample the presence of one or more of a plurality of different target nucleic acids. This invention also provides related kits

This application claims benefit of U.S. Provisional Application No. 60/566,967, filed Apr. 29, 2004, the contents of which are hereby incorporated by reference.

The invention disclosed herein was made with Government support under grant no. AI51292 from the National Institutes of Health. Accordingly, the U.S. Government has certain rights in this invention.

Throughout this application, various publications are referenced. Full citations for these references may be found at the end of the specification immediately preceding the claims. The disclosures of these publications in their entireties are hereby incorporated by reference into this application to more fully describe the state of the art to which this invention pertains.

### BACKGROUND OF THE INVENTION

Establishing a causal relationship between infection with a virus and a specific disease may be complex. In most acute viral diseases, the responsible agent is readily implicated because it replicates at high levels in the affected tissue at the time the disease is manifest, morphological changes consistent with infection are evident, and the agent is readily cultured with standard microbiological techniques. In contrast, implication of viruses in chronic diseases may be confounded because persistence requires restricted gene expression, classical hallmarks of infection are absent, and/or Methods for cloning nucleic acids of microbial pathogens directly from clinical specimens offer new opportunities to investigate microbial associations in chronic diseases. The power of these methods is that they can succeed where methods for pathogen identification through serology or cultivation may fail due to absence of specific reagents or fastidious requirements for agent replication. Over the past decade, the application of molecular pathogen discovery methods resulted in identification of novel agents associated with both acute and chronic diseases, including Borna disease virus, Hepatitis C virus, Sin Nombre virus, HHV-6, HHV-8, Bartonella henselae, and Tropherema whippeli.

Various methods are employed or proposed for cultivation-independent characterization of infectious agents. These can be broadly segregated into methods based on direct analysis of microbial nucleic acid sequences. (e.g., cDNA microarrays, consensus PCR, representational difference analysis, differential display), direct analysis of microbial protein sequences (e.g., mass spectrometry), immunological systems for microbe detection (e.g., expression libraries, phage display) and host response profiling. A comprehensive program in pathogen discovery would need to exploit most, if not all, of these technologies.

The decision to employ a specific method is guided by the clinical features, epidemiology, and spectrum of potential pathogens to be implicated. Expression libraries, comprised of cDNAs or synthetic peptides, may be useful tools in the event that large quantities of acute and convalescent sera or cerebrospinal fluid are available for screening purposes; however, the approach is cumbersome, labor-intensive, and success is dependent on the presence of a specific, high affinity humoral immune response. The utility of host response mRNA profile analysis has been demonstrated in several in vitro paradigms and some inbred animal models; nonetheless, it is important to formally consider the possibility that a variety of organisms may activate similar cascades of chemokines, cytokines, and other soluble factors that influence host gene expression to produce what are likely to be convergent gene expression profiles. Thus, at least in virology, it is prudent to explore complementary methods for pathogen identification based on agent-encoded nucleic acid motifs. Given the potential for high density printing of microarrays, it is feasible to design slides or chips decorated with both host and pathogen targets. This would provide an unprecedented opportunity to simultaneously survey host response mRNA profiles and viral flora, providing insights into microbial pathogenesis not apparent with either method of analysis alone.

Representational difference analysis (RDA) is an important tool for pathogen identification and discovery. However, RDA is a subtractive cloning method for binary comparisons of nucleic acid populations. Thus, although ideal for analysis of cloned cells or tissue samples that differ only in a single variable of interest, RDA is less well suited to investigation of syndromes wherein infection with any of several different pathogens results in similar clinical manifestations, or infection is not invariably associated with disease. An additional caveat is that because the method is dependent upon the presence of a limited number of restriction sites, RDA is most likely to succeed for agents with large genomes. Indeed, in this context, it is noteworthy that the two viruses detected by RDA in the listing above were herpesviruses.

Consensus PCR (cPCR) has been a remarkably productive tool for biology. In addition to identifying pathogens, particularly genomes of prokaryotic pathogens, this method has facilitated identification of a wide variety of host molecules, including cytokines, ion channels, and receptors. Nonetheless, until recently, a difficulty in applying cPCR to pathogen discovery in virology has been that it is difficult to identify conserved viral sequences of sufficient length to allow cross-hybridization, amplification, and discrimination using traditional cPCR format. While this may not be problematic when one is targeting only a single virus family, the number of assays required becomes infeasible when preliminary data are insufficient to allow a directed, limited analysis.

Real-time PCR methods have significantly changed diagnostic molecular microbiology by providing rapid, sensitive, specific tools for detecting and quantitating genetic targets. Because closed systems are employed, real-time PCR is less likely than nested PCR to be confounded by assay contamination due to inadvertent aerosol introduction of amplicon/positive control/cDNA templates that can accumulate in diagnostic laboratories. The specificity of real time PCR is both a strength and a limitation. Although the potential for false positive signal is low so is the utility of the method for screening to detect related but not identical genetic targets. Specificity in real-time PCR is provided by two primers (each approximately 20 matching nucleotides (nt) in length) combined with a specific reporter probe of about 27 nt. The constraints of achieving hybridization at all three sites may confound detection of diverse, rapidly evolving microbial genomes such as those of single-stranded RNA viruses. These constraints can be compensated in part by increasing numbers of primer sets accommodating various templates. However, because real-time PCR relies on fluorescent reporter dyes, the capacity for multiplexing is limited to the number of emission peaks that can be unequivocally separated. At present up to four dyes can be identified simultaneously. Although the repertoire may increase, it will not likely change dramatically.

### SUMMARY OF THE INVENTION

This invention provides a method for simultaneously detecting in a sample the presence of one or more of a plurality of different target nucleic acids comprising the steps of:
(a) contacting the sample with a plurality of nucleic acid primers simultaneously and under conditions permitting, and for a time sufficient for, primer extension to occur, wherein (i) for each target nucleic acid at least one predetermined primer is used which is specific for that target nucleic acid, (ii) each primer has a mass tag of predetermined size bound thereto via a labile bond, and (iii) the mass tag bound to any primer specific for one target-nucleic acid has a different mass than the mass tag bound to any primer specific for any other target nucleic acid;
(b) separating any unextended primers from any extended primers;
(c) simultaneously cleaving the mass tags from any extended primers; and
(d) simultaneously determining the presence and sizes of any mass tags so cleaved,
   wherein the presence of a cleaved mass tag having the same size as a mass tag of predetermined size previously bound to a predetermined primer indicates the presence in the sample of the target nucleic acid specifically recognized by that predetermined primer.

This invention further provides the instant method, wherein the method detects the presence in the sample of 10 or more, 50 or more, 100 or more, or 200 or more different target nucleic acids. This invention further provides the instant method, wherein the sample is contacted with 4 or more, or 10 or more, or 50 or more, or 100 or more, or 200 or more different primers.

This invention further provides the instant method, wherein one or more primers comprises the sequence set forth in one of SEQ ID NOs:1-96, and 98-101. This invention further provides the instant method, wherein at least two different primers are specific for the same target nucleic acid. This invention further provides the instant method, wherein a first primer is a forward primer for the target nucleic acid and a second primer is a reverse primer for the same target nucleic acid.

This invention further provides the instant method, wherein the mass tags bound to the first and second primers are of the same size. This invention further provides the instant method, wherein the mass tags bound to the first and second primers are of a different size.

This invention further provides the instant method, wherein at least one target nucleic acid is from a pathogen.

This invention further provides the instant method, wherein the presence and size of any cleaved mass tag is determined by mass spectrometry. This invention further provides the instant method, wherein the mass spectrometry is selected from the group consisting of atmospheric pressure chemical ionization mass spectrometry, electrospray ionization mass spectrometry, and matrix assisted laser desorption ionization mass spectrometry.

### BRIEF DESCRIPTION OF THE FIGURES

FIG. 1: This figure shows the structure of mass tag precursors and four photoactive mass tags.

FIG. 2: This figure shows an ACPI mass spectrum of mass tag precursors for digital virus detection.

FIG. 3: This figure shows DNA sequencing sample preparation for MS analysis using biotinylated dideoxynucleotides and a streptavidin coated solid phase.

FIG. 4: This figure shows a mass spectrum from Sanger sequencing reactions using dd(A, G, C)TP-11-biotin and ddTTP-16-biotin.

FIG. 5: This figure shows synthesis of NHS ester of one mass tag for tagging amino-primer (SEQ ID NO:97).

FIG. 6: This figure shows the general structure of mass tags and photocleavage mechanism to release the mass tags from DNA for MS detection.

FIG. 7: This figure shows four mass tagged biotinylated ddNTPs.

FIG. 8: This figure shows the structure of four mass tag precursors and the four photoactive mass tags.

FIG. 9: This figure shows APCI mass spectra for four mass tags after cleavage from primers. 2-nitrosacetophenone, m/Z 150; 4 fluoro-2-nitrosacetophenone, m/z 168; 5-methoxy-2-nitrosacetophenone, m/z 180; and 4,5-dimethoxy-2-nitrosacetophenone.

FIG. 10: This figure shows four mass tag-labeled DNA molecules.

FIG. 11: This figure shows differential real-time PCR for HCoV SARS, OC43, and 229E.

FIG. 12: This figure shows 58 tags cleaved from oligonucleotides and detected using ACPI-MS. Each peak represents a different tag structure as a unique signature of the oligonucleotide it was originally attached to.

FIG. 13: This figure shows singleplex mass tag PCR for (1) influenza A virus matrix protein, (2) human coronavirus SARS, (3) 229E, (4) OC43, and (5) the bacterial agent M. pneumoniae. (6) shows a 100 bp ladder.

FIG. 14: This figure shows mass spectrum representative of data collected using a miniaturized cylindrical ion trap mass analyzer coupled with a corona discharge ionization source.

FIG. 15: This figure shows mass spectrum of perfluoro-dimethylcyclohexane collected on a prototype atmospheric sampling glow discharge ionization source.

FIG. 16: This figure shows the sensitivity of a 21-plex mass tag PCR. Dilutions of cloned gene target standards (10 000, 1 000, 500, 100 molecules/assay) diluted in human placenta DNA were analyzed by mass tag PCR. Each reaction mix contained 2× Multiplex PCR Master Mix (Qiagen), the indicated standard and 42 primers at 1×nM concentration labeled with different mass tags. Background in reactions without standard (no template control, 12.5 ng human DNA) was subtracted and the sum of Integrated Ion Current for both tags was plotted.

FIG. 17: This figure shows analysis of clinical specimens; respiratory infection. RNA from clinical specimens was extracted by standard procedures and reverse transcribed into cDNA (Superscript RT system, Invitrogen, Carlsbad, Calif.; 20 ul volume). Five microliter of reaction was then subjected to mass tag PCR.

FIG. 18: This figure shows multiplex mass tag PCR analysis of six human respiratory specimens. Mass tag primer sets employed in a single tube assay are indicated at the bottom of the figure.

FIG. 19: This figure shows structures of MASSCODE tags.

FIG. 20: This figure shows differential real-time PCR for West Nile virus and St. Louis encephalitis virus.

FIGS. 21A-21B: (A) This figure shows serial dilutions of plasmid standards (5×10⁵, 5×10⁴, 5×10³, 5×10², 5×10¹, and 5×10⁰) for RSV group A, RSV group B, Influenza A, HCoV-SARS, HCoV-229E, HCoV-OC43, and M. pneumoniae were each analyzed by mass tag PCR in a multiplex format. (B) This figure shows simultaneous detection of multiple targets in multiplex format using mixtures of two templates per assay (5×10⁴ copies each): HCoV-SARS and M. pneumoniae, HCoV-229E and M. pneumoniae, HCoV-OC43 and M. pneumoniae, and HCoV-229E and HCoV-OC43.

FIG. 22: This figure shows a schematic of the mass tag PCR procedure.

FIG. 23: Thus figure shows identification of various infections using masscode tags.

### DETAILED DESCRIPTION OF THE INVENTION

Terms

As used herein, and unless stated otherwise, each of the following terms shall have the definition set forth below.

“Mass tag” shall mean any chemical moiety (i) having a fixed mass, (ii) affixable to a nucleic acid, and (iii) whose mass is determinable using mass spectrometry. Mass tags include, for example, chemical moieties such as small organic molecules, and have masses which range, for example, from 100 Da to 2500 Da.

“Nucleic acid” shall mean any nucleic acid molecule, including, without limitation, DNA, RNA and hybrids thereof. The nucleic acid bases that form nucleic acid molecules can be the bases A, C, G, T and U, as well as derivatives thereof. Derivatives of these bases are well known in the art, and are exemplified in PCR Systems, Reagents and Consumables (Perkin Elmer Catalogue 1996-1997, Roche Molecular Systems, Inc., Branchburg, N.J., USA).

“Pathogen” shall mean an organic entity including, without limitation, viruses and bacteria, known or suspected to be involved in the pathogenesis of a disease state in an organism such as an animal or human.

“Sample” shall include, without limitation, a biological sample derived from an animal or a human, such as cerebro-spinal fluid, lymph, blood, blood derivatives (e.g. sera), liquidized tissue, urine and fecal material.

“Simultaneously detecting”, with respect to the presence of target nucleic acids in a sample, means determining, in the same reaction vessels(s), whether none, some or all target nucleic acids are present in the sample. For example, in the instant method of simultaneously detecting in a sample the presence of one or more of 50 target nucleic acids, the presence of each of the 50 target nucleic acids will be determined simultaneously, so that results of such detection could be, for example, (i) none of the target nucleic acids are present, (ii) five of the target nucleic acids are present, or (iii) all 50 of the target nucleic acids are present.

“Specific”, when used to describe a primer in relation to a target nucleic acid, shall mean that, under primer extension-permitting conditions, the primer specifically binds to a portion of the target nucleic acid and is extended.

“Target nucleic acid” shall mean a nucleic acid whose presence in a sample is to be detected by any of the instant methods.

“5-UTR” shall mean the 5′-end untranslated region of a nucleic that encodes a protein.

The following abbreviations shall have the meanings set forth below: “A” shall mean Adenine; “bp” shall mean base pairs; “C” shall mean Cytosine; “DNA” shall mean deoxyribonucleic acid; “G” shall mean Guanine; “mRNA” shall mean messenger ribonucleic acid; “RNA” shall mean ribonucleic acid; “PCR” shall mean polymerase chain reaction; “T” shall mean Thymine; “U” shall mean Uracil; “Da” shall mean dalton.

Finally, with regard to the embodiments of this invention, where a numerical range is stated, the range is understood to encompass the embodiments of each and every integer between the lower and upper numerical limits. For example, the numerical range from 1 to 5 is understood to include 1, 2, 3, 4, and 5.

### EMBODIMENTS OF THE INVENTION

To address the need for enhanced multiplex capacity in diagnostic molecular microbiology we have established a PCR platform based on mass tag reporters that are easily distinguished in Mass Spectrometry (MS) as discrete signal peaks. Major advantages of the PCR/MS system include: (1) hybridization to only two sites is required (forward and reverse primer binding sites) vs real time PCR where an intermediate third oligonucleotide is used (probe binding site); this enhances flexibility in primer design; (2) tried and proven consensus PCR primers can be adapted to PCR/MS; this reduces the time and resources that must be invested to create new reagents and assay controls; (3) the large repertoire of tags allows highly multiplexed assays; additional tags can be easily synthesized to allow further complexity; and (4) sensitivity of real time PCR is maintained. We view PCR/MS as a tool with which to rapidly screen clinical materials for the presence of candidate pathogens. Thereafter, targeted secondary tests, including real time PCR, can be used to quantitate microbe burden and pursue epidemiologic studies.

Specifically, this invention provides a method for simultaneously detecting in a sample the presence of one or more of a plurality of different target nucleic acids comprising the steps of:
(a) contacting the sample with a plurality of nucleic acid primers simultaneously and under conditions permitting, and for a time sufficient for, primer extension to occur, wherein (i) for each target nucleic acid at least one predetermined primer is used which is specific for that target nucleic acid, (ii) each primer has a mass tag of predetermined size bound thereto via a labile bond, and (iii) the mass tag bound to any primer specific for one target nucleic acid has a different mass than the mass tag bound to any primer specific for any other target nucleic acid;
(b) separating any unextended primers from any extended primers;
(c) simultaneously cleaving the mass tags from any extended primers; and
(d) simultaneously determining the presence and sizes of any mass tags so cleaved,
   wherein the presence of a cleaved mass tag having the same size as a mass tag of predetermined size previously bound to a predetermined primer indicates the presence in the sample of the target nucleic acid specifically recognized by that predetermined primer.

In one embodiment of the instant method, the method detects the presence in the sample of 10 or more different target nucleic acids. In another embodiment, the method detects the presence in the sample of 50 or more different target nucleic acids. In a further embodiment, the method detects the presence in the sample of 100 or more different target nucleic acids. In a further embodiment, the method detects the presence in the sample of 200 or more different target nucleic acids.

In one embodiment of the instant method, the sample is contacted with 4 or more different primers. In another embodiment, the sample is contacted with 10 or more different primers. In a further embodiment, the sample is contacted with 50 or more different primers. In a further embodiment, the sample is contacted with 100 or more different primers. In yet a further embodiment, the sample is contacted with 200 or more different primers.

In one embodiment of the instant method, one or more primers comprises the sequence set forth in one of SEQ ID NOs:1-96, and 98-101.

In another embodiment of the instant method, at least two different primers are specific for the same target nucleic acid. For example, in one embodiment a first primer is a forward primer for the target nucleic acid and a second primer is a reverse primer for the same target nucleic acid. In this example, the mass tags bound to the first and second primers can be of the same size or of different sizes. In another embodiment, a first primer is directed to a 5′-UTR of the target nucleic acid and a second primer is directed to a 3D polymerase region of the target nucleic acid.

In one embodiment of the instant method, wherein each primer is from 15 to 30 nucleotides in length. In another embodiment, each mass tag has a molecular weight of from 100 Da to 2,500 Da. In a further embodiment, the labile bond is a photolabile bond, such as a photolabile bond cleavable by ultraviolet light.

In another embodiment of the instant method, at least one target nucleic acid is from a pathogen. Pathogens include, without limitation, B. anthracis, a Dengue virus, a West Nile virus, Japanese encephalitis virus, St. Louis encephalitis virus, Yellow Fever virus, La Crosse virus, California encephalitis virus, Rift Valley Fever virus, CCHF virus, VEE virus, EEE virus, WEE virus, Ebola virus, Marburg virus, LCMV, Junin virus, Machupo virus, Variola virus, SARS corona virus, an enterovirus, an influenza virus, a parainfluenza virus, a respiratory syncytial virus, a bunyavirus, a flavivirus, and an alphavirus.

In another embodiment, the pathogen is a respiratory pathogen. Respiratory pathogens include, for example, respiratory syncytial virus A, respiratory syncytial virus B, Influenza A (N1), Influenza A (N2), Influenza A (M), Influenza A (H1), Influenza A (H2), Influenza A (H3), Influenza A (H5), Influenza B, SARS coronavirus, 229E coronavirus, OC43 coronavirus, Metapneumovirus European, Metapneumovirus Canadian, Parainfluenza 1, Parainfluenza 2, Parainfluenza 3, Parainfluenza 4A, Parainfluenza 4B, Cytomegalovirus, Measles virus, Adenovirus, Enterovirus, M. pneumoniae, L. pneumophilae, and C. pneumoniae.

In a further embodiment, the pathogen is an encephalitis-inducing pathogen. Encephalitis-inducing pathogens include, for example, West Nile virus, St. Louis encephalitis virus, Herpes Simplex virus, HIV 1, HIV 2, N. meningitides, S. pneumoniae, H. influenzae, Influenza B, SARS coronavirus, 229E-CoV, OC43-CoV, Cytomegalovirus, and a Varicella Zoster virus. In a further embodiment, the pathogen is a hemorrhagic fever-inducing pathogen. In a further embodiment, the sample is a forensic sample, a food sample, blood, or a derivative of blood, a biological warfare agent or a suspected biological warfare agent.

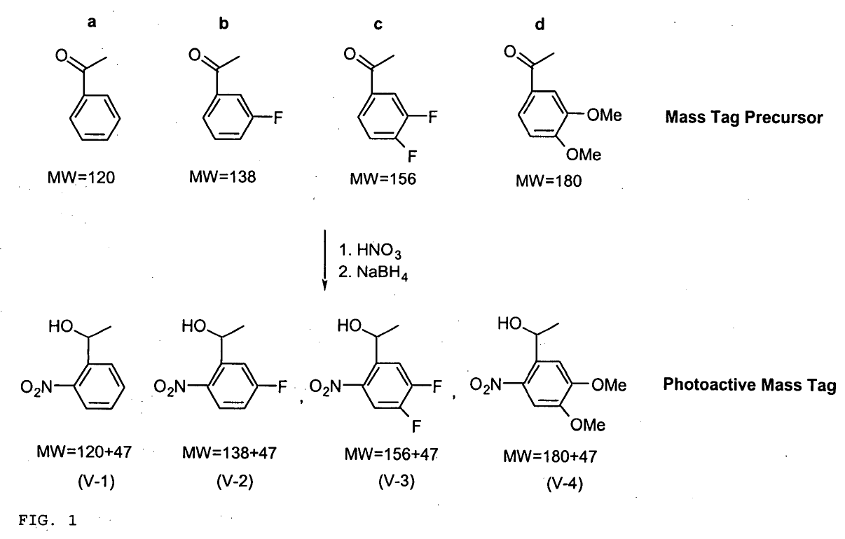

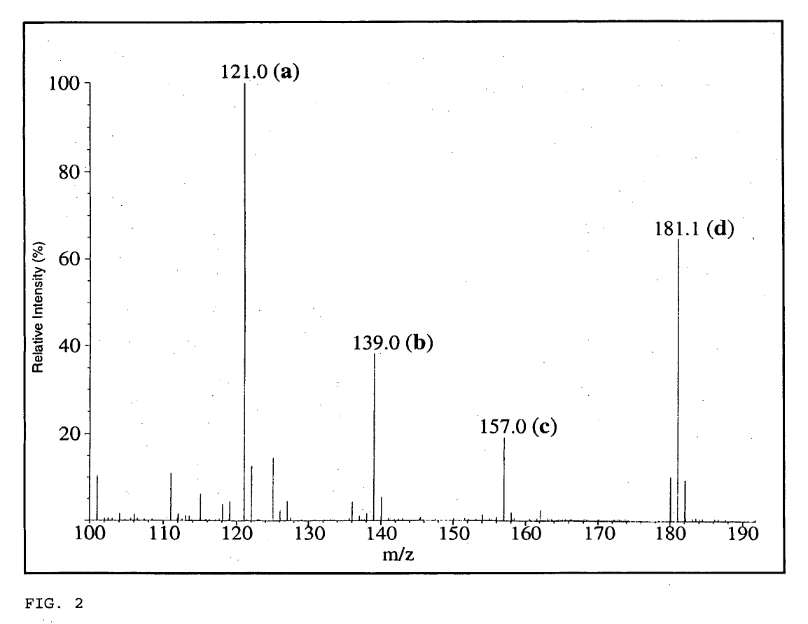

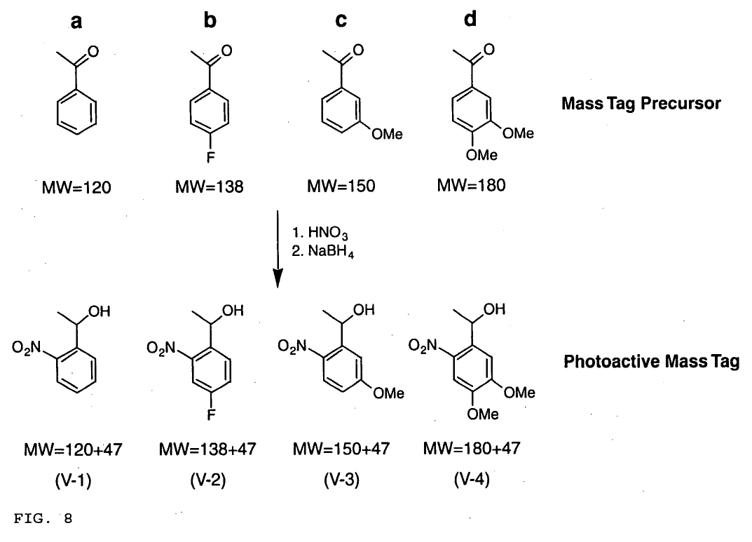

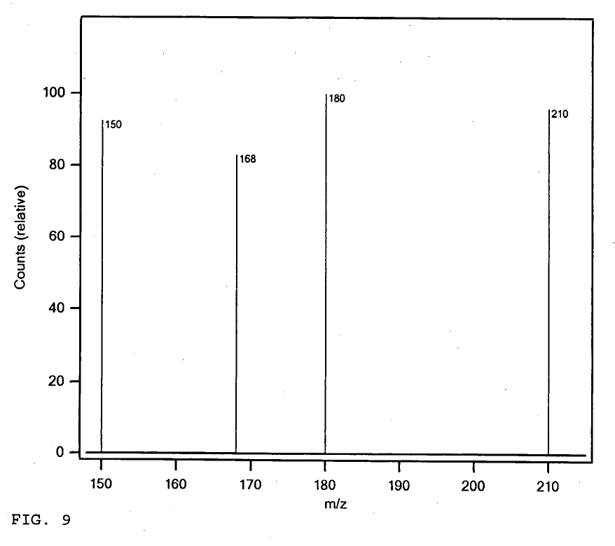

In one embodiment of the instant method, the mass tag is selected from the group consisting of structures V1 to V4 of FIG. 1 or FIG. 8.

In another embodiment of the instant method, the presence and size of any cleaved mass tag is determined by mass spectrometry. Mass spectrometry includes, for example, atmospheric pressure chemical ionization mass spectrometry, electrospray ionization mass spectrometry, and matrix assisted laser desorption ionization mass spectrometry.

In one embodiment of the instant method, the target nucleic acid is a ribonucleic acid. In another embodiment, the target nucleic acid is a deoxyribonucleic acid. In a further embodiment, the target nucleic acid is from a viral source.

This invention provides a kit for simultaneously detecting in a sample the presence of one or more of a plurality of different target nucleic acids comprising a plurality of nucleic acid primers wherein (i) for each target nucleic acid at least one predetermined primer is used which is specific for that target nucleic acid, (ii) each primer has a mass tag of predetermined size bound thereto via a labile bond, and (iii) the mass tag bound to any primer specific for one target nucleic acid has a different mass than the mass tag bound to any primer specific for any other target nucleic acid.

This invention also provides a kit for simultaneously detecting in a sample the presence of one or more of a plurality of different target nucleic acids comprising (a) a plurality of nucleic acid primers wherein (i) for each target nucleic acid at least one predetermined primer is used which is specific for that target nucleic acid, (ii) each primer has a mass tag of predetermined size bound thereto via a labile bond, and (iii) the mass tag bound to any primer specific for one target nucleic acid has a different mass than the mass tag bound to any primer specific for any other target nucleic acid; and (b) a mass spectrometer.

This invention further provides a kit for simultaneously detecting in a sample the presence of one or more of a plurality of different target nucleic acids comprising (a) a plurality of nucleic acid primers wherein (i) for each target nucleic acid at least one predetermined primer is used which is specific for that target nucleic acid, (ii) each primer has a mass tag of predetermined size bound thereto via a labile bond, and (iii) the mass tag bound to any primer specific for one target nucleic acid has a different mass than the mass tag bound to any primer specific for any other target nucleic acid, and (b) instructions for use.

Finally, this invention provides a kit for simultaneously detecting in a sample the presence of one or more of a plurality of different target nucleic acids comprising (a) a plurality of nucleic acid primers wherein (i) for each target nucleic acid at least one predetermined primer is used which is specific for that target nucleic acid, (ii) each primer has a mass tag of predetermined size bound thereto via a labile bond, and (iii) the mass tag bound to any primer specific for one target nucleic acid has a different mass than the mass tag bound to any primer specific for any other target nucleic acid; (b) a mass spectrometer; and (c) instructions for simultaneously detecting in a sample the presence of one or more of a plurality of different target nucleic acids using the primers and the mass spectrometer.

This invention will be better understood by reference to the Experimental Details which follow, but those skilled in the art will readily appreciate that the specific experiments detailed are only illustrative of the invention as described more fully in the claims which follow thereafter.

### EXPERIMENTAL DETAILS

### Example 1

Abbreviations: 5′-UTR, 5′-untranslated region; ALS, Amyotrophic Lateral Sclerosis; APCI, atmospheric pressure chemical ionization; ESI, electrospray ionization; PCR, polymerase chain reaction; MALDI-TOF, matrix assisted laser desorption ionization time of flight; MS, mass spectrometry

Background

Establishing a causal relationship between infection with a virus and a specific disease may be complex. In most acute viral diseases, the responsible agent is readily implicated because it replicates at high levels in the affected tissue at the time the disease is manifest, morphological changes consistent with infection are evident, and the agent is readily cultured with standard microbiological techniques. In contrast, implication of viruses in chronic diseases may be confounded because persistence requires restricted gene expression, classical hallmarks of infection are absent, and/or mechanisms of pathogenesis are indirect or subtle. Methods for cloning nucleic acids of microbial pathogens directly from clinical specimens offer new opportunities to investigate microbial associations in chronic diseases (21). The power of these methods is that they can succeed where methods for pathogen identification through serology or cultivation may fail due to absence of specific reagents or fastidious requirements for agent replication. Over the past decade, the application of molecular pathogen discovery methods resulted in identification of novel agents associated with both acute and chronic diseases, including Borna disease virus, Hepatitis C virus, Sin Nombre virus, HHV-6, HHV-8, Bartonella henselae, and Tropherema whippeli (5-7, 17, 19, 22, 23, 27).

Various methods are employed or proposed for cultivation-independent characterization of infectious agents. These can be broadly segregated into methods based on direct analysis of microbial nucleic acid sequences (e.g., cDNA microarrays, consensus PCR, representational difference analysis, differential display), direct analysis of microbial protein sequences (e.g., mass spectrometry), immunological systems for microbe detection (e.g., expression libraries, phage display) and host response profiling. A comprehensive program in pathogen discovery will need to exploit most, if not all, of these technologies.

The decision to employ a specific method is guided by the clinical features, epidemiology, and spectrum of potential pathogens to be implicated. Expression libraries, comprised of cDNAs or synthetic peptides, may be useful tools in the event that large quantities of acute and convalescent sera or cerebrospinal fluid are available for screening purposes; however, the approach is cumbersome, labor-intensive, and success is dependent on the presence of a specific, high affinity humoral immune response. The utility of host response mRNA profile analysis has been demonstrated in several in vitro paradigms and some inbred animal models (8, 26, 30); nonetheless, it is important to formally consider the possibility that a variety of organisms may activate similar cascades of chemokines, cytokines, and other soluble factors that influence host gene expression to produce what are likely to be convergent gene expression profiles. Thus, at least in virology, it is prudent to explore complementary methods for pathogen identification based on agent-encoded nucleic acid motifs. Given the potential for high density printing of microarrays, it is feasible to design slides or chips decorated with both host and pathogen targets. This would provide an unprecedented opportunity to simultaneously survey host response mRNA profiles and viral flora, providing insights into microbial pathogenesis not apparent with either method of analysis alone. Representational difference analysis (RDA) is an important tool for pathogen identification and discovery. However, RDA is a subtractive cloning method for binary comparisons of nucleic acid populations (12, 18). Thus, although ideal for analysis of cloned cells or tissue samples that differ only in a single variable of interest, RDA is less well suited to investigation of syndromes wherein infection with any of several different pathogens results in similar clinical manifestations, or infection is not invariably associated with disease. An additional caveat is that because the method is dependent upon the presence of a limited number of restriction sites, RDA is most likely to succeed for agents with large genomes. Indeed, in this context, it is noteworthy that the two viruses detected by RDA in the listing above (see first paragraph) were herpesviruses (5, 6). Consensus PCR (cPCR) has been a remarkably productive tool for biology. In addition to identifying pathogens, particularly genomes of prokaryotic pathogens, this method has facilitated identification of a wide variety of host molecules, including cytokines, ion channels, and receptors. Nonetheless, until recently, a difficulty in applying cPCR to pathogen discovery in virology has been that it is difficult to identify conserved viral sequences of sufficient length to allow cross-hybridization, amplification, and discrimination using traditional cPCR format. While this may not be problematic when one is targeting only a single virus family, the number of assays required becomes infeasible when preliminary data are insufficient to allow a directed, limited analysis. To address this issue, we adapted cPCR to Differential Display, a PCR-based method for simultaneously displaying the genetic composition of multiple sample populations in an acrylamide gel format (16). This hybrid method, domain-specific differential display (DSDD), employs short, degenerate primer sets designed to hybridize to viral genes representing larger taxonomic categories than can be resolved in cPCR. The major advantages to this approach are: (i) reduction in numbers of reactions required to identify genomes of known viruses, and (ii) potential to detect viruses less closely related to known viruses than those found through cPCR. The differential display format also permits identification of syndrome-specific patterns of gene expression (host and pathogen) that need not be present in all clinical samples. Additionally, because multiple samples can be analyzed in side-by-side comparisons, DSDD allows examination of the timecourse of gene expression patterns. Lastly, recent experience with isolation of the West Nile virus responsible for the outbreak of encephalitis in New York in the summer of 1999 indicates that DSDD may be advantageous in instances where template is suboptimal due to degradation (e.g., postmortem field specimens).

The development and application of sensitive high throughput methods for detecting a wide range of viruses is anticipated to provide new insights into the pathogenesis of chronic diseases. We are funded through AI51292 to support these objectives by establishing DNA microarray, multiplexed bead-based flow cytometric (MB-BFC) and domain specific differential display (DSDD) assay platforms for viral surveillance and discovery in chronic diseases. Each of these methods has its strengths; however, none is ideal. Microarrays provide a platform wherein one can simultaneously query thousands of microbial and host gene targets but lack sensitivity and are difficult to modify as new targets are identified. Bead-based arrays are flexible but similar in sensitivity to microarrays.

Domain specific differential display is sensitive and flexible but labor intensive. Real time PCR (not a component of our original application but useful to note for purposes of method comparisons), is rapid and sensitive, but cannot be used for broad range detection of viral sequences, because of stringent sequence constraints for the three oligonucleotides comprising the system (two primers, one probe).

Mass-Tag PCR would integrate PCR and mass spectrometry (MS) into a stable and sensitive digital assay platform. It is similar in sensitivity and efficiency to real time PCR but provides the advantages of simultaneous detection and discrimination of multiple targets, due to less stringent constraints on primer selection. Additionally, whereas multiplexing is limited in real time PCR by overlapping fluorescence emission spectra, Mass-Tag PCR allows discrimination of a large repertoire of mass tags with molecular weights between 150 and 2500 daltons.

In Mass-Tag PCR, virus identity is be defined by the presence of label of a specific molecular weight associated with an amplification product. Primers are be designed such that the tag can be cleaved by irradiation with UV light. Following PCR, the amplification product can be immobilized on a solid support and excess soluble primer removed. After cleavage by UV irradiation (˜350 nm), the released tag will be analyzed by mass spectrometry. Detection is sensitive, fast, independent of DNA fragment length, and ideally suited to the multiplex format required to survey clinical materials for infection with a wide range of infectious agents.

Results

Mass spectrometry (MS) is a rapid, sensitive method for detection of small molecules. With the development of new ionization techniques such as matrix assisted laser desorption ionization (MALDI) and electrospray ionization (ESI), mass spectrometry has become an indispensable tool in many areas of biomedical research. Although these ionization methods are suitable for the analysis of bioorganic molecules, such as peptides and proteins, improvements in both detection and sample preparation will be required before mass spectrometry can be used to directly detect long DNA fragments. A major confound in exploiting MS for genetic investigation has been that long DNA molecules are fragmented during the analytic process. The mass tag approach overcomes this limitation by detecting small stable mass tags that serve as signatures for specific DNA sequences rather than the DNA sequences themselves.

Atmospheric pressure chemical ionization (APCI) has advantages over ESI and MALDI for some applications. Because buffer and inorganic salts impact ionization efficiency, performance in ESI is critically dependent upon sample preparation conditions. In MALDI, matrix must be added prior to sample introduction into the mass spectrometer; speed is often limited by the need to search for an ideal irradiation spot to obtain interpretable mass spectra. APCI requires neither desalting nor mixing with matrix to prepare crystals on a target plate. Therefore in APCI, mass tag solutions can be injected directly. Because mass tags are volatile and have small mass values, they are easily detected by APCI ionization with high sensitivity. The APCI mass tag system is easily scaled up for high throughput operation.

We have established methods for synthesis and APCI analysis of mass tags coupled to DNA fragments. Precursors of four mass tags [(a) acetophenone; (b) 3-fluoroacetophenone; (c) 3,4-difluoroacetophenone; and (d) 3,4-dimethoxyacetophenone] are shown in FIG. 1. Upon nitration and reduction, the photoactive tags are produced and used to code for the identity of up to four different primer pairs (or target sequences). In a simulation experiment, we have obtained clean APCI mass spectra for the 4 mass tag precursors (a, b, c, d) as shown in FIG. 2. The peak with m/z of 121 is a, 139 is b, 157 is c and 181 is d. This result indicates that the 4 compounds we designed as mass tags are stable and produce discrete high resolution digital data in an APCI mass spectrometer. In the research described below, each of the unique m/z from each mass tag translates to the identity of a viral sequence (V) [Tag-1 (m/z, 150)=V-1; Tag-2 (m/z, 168)=V-2; Tag-3 (m/z, 186)=V-3; Tag-4 (m/z, 210)=V-4]. A variety of functional groups can be introduced to the mass tag parent structure for generating a large number of mass tags with different molecular weights. Thus, a library of primers labeled with mass tags that can discriminate between hundreds of viral sequence targets.

DNA Sequencing with Biotinylated Dideoxynucleotides on a Mass Spectrometer

PCR amplification can be nonspecific; thus, products are commonly sequenced to verify their identity as bona fide targets. Here we apply the rapidity and sensitivity of mass tag analyses to direct MS-sequencing of PCR amplified transcripts.

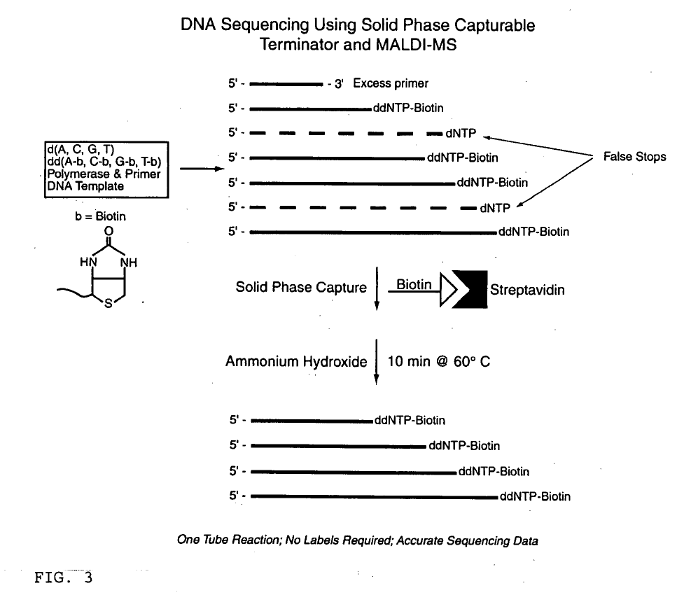

MALDI-TOF MS has recently been explored widely for DNA sequencing. The Sanger dideoxy procedure (25) is used to generate the DNA sequencing fragments. The mass resolution in theory can be as good as one dalton; however, in order to obtain accurate measurement of the mass of the sequencing DNA fragments, the samples must be free from alkaline and alkaline earth salts and falsely stopped DNA fragments (fragments terminated at dNTPs instead of ddNTPs). Our method for preparing DNA sequencing fragments using biotinylated dideoxynucleotides and a streptavidin-coated solid phase is shown in FIG. 3. DNA template, dNTPs (A, C, G, T) and ddNTP-biotin (A-b, C-b, G-b, T-b), primer and DNA polymerase are combined in one tube. After polymerase extension and termination reactions, a series of DNA fragments with different lengths are generated. The sequencing reaction mixture is then incubated for a few minutes with a streptavidin-coated solid phase. Only the DNA sequencing fragments that are terminated with biotinylated dideoxynucleotides at the 3′ end are captured on the solid phase. Excess primers, falsely terminated DNA fragments, enzymes and all other components from the sequencing reaction are washed away. The biotinylated DNA sequencing fragments are then cleaved off the solid phase by disrupting the interaction between biotin and streptavidin using ammonium hydroxide or formamide to obtain a pure set of DNA sequencing fragments. These fragments are then mixed with matrix (3-hydroxypicolinic acid) and loaded onto a mass spectrometer to produce accurate mass spectra of the DNA sequencing fragments. Since each type of nucleotide has a unique molecular mass, the mass difference between adjacent peaks of the mass spectra gives the sequence identity of the nucleotides. In DNA sequencing with mass spectrometry, the purity of the samples directly affects the quality of the obtained spectra. Excess primers, salts, and fragments that are prematurely terminated in the sequencing reactions (false stops) will create extra noise and extraneous peaks (11). Excess primers can also dimerize to form high molecular weight species that give a false signal in mass spectrometry (29). False stops occur in DNA sequencing reaction when a deoxynucleotide rather than a dideoxynucleotide terminates a sequencing fragment. A deoxynucleotide terminated false stop has a mass difference of 16 daltons compared with its dideoxy counterpart. This mass difference is identical to the difference between adenine and guanine. Thus, false stops can be misinterpreted or interfere with existing peaks in the mass spectra. Our method is designed to eliminate these confounds. We previously established a procedure for accurately sequencing DNA using fluorescent dye-labeled primers and biotinylated dideoxynucleotides. In this procedure, accurate and clean DNA sequencing data were obtained by removing falsely stopped fragments prior to analysis through use of an intermediate purification step on streptavidin-coated magnetic beads (13, 14).

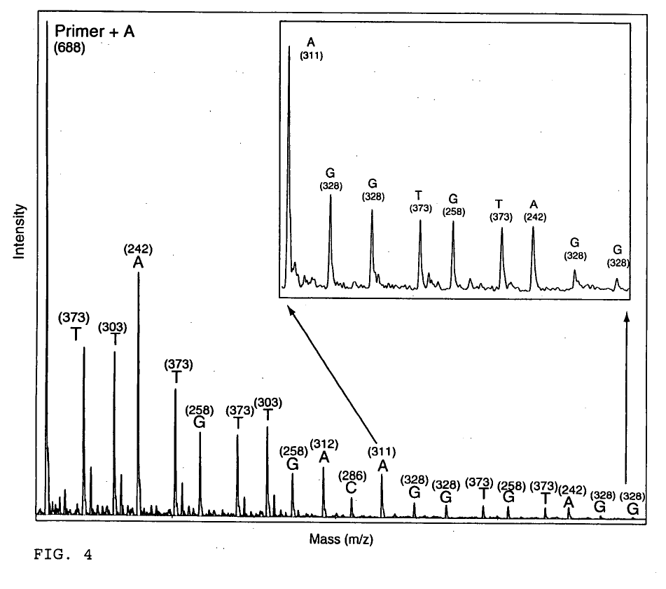

Sequencing experiments for a 55 bp synthetic template using MALDI-TOF mass spectrometry were recently performed (9). Four commercially available biotinylated dideoxynucleotides ddATP-11-biotin, ddGTP-11-biotin, ddCTP-11-biotin and ddTTP-11-biotin (NEN, Boston) were used to produce the sequencing ladder in a single tube by cycle sequencing. Clean sequence peaks were obtained on the mass spectra, with the first peak being primer extended by one biotinylated dideoxynucleotide. Although the identity of A and G residues were determined unambiguously, C and T could not be differentiated because the one dalton mass difference between the ddCTP-11-biotin and ddTTP-11-biotin cannot be consistently resolved by using the current mass detector for DNA fragments. Nonetheless, these results confirmed that clean sequencing ladders can be obtained by capture/release of DNA sequencing fragments with biotin located on the 3′ dideoxy terminators. The procedure has been improved by using biotinylated ddTTPs that have large mass differences in comparison to ddCTP-11-biotin. Pairing ddTTP-16-biotin (Enzo, Boston), which has a large mass difference in comparison to ddCTP-11-biotin, with ddATP-11-biotin, ddCTP-11-biotin, and ddGTP-11-biotin, allowed unambiguous sequence determination in the mass spectra (FIG. 4). Mass spectrum from Sanger sequencing reactions using dd(A,G,C)TP-11-biotin and ddTTP-16-biotin. All four bases are unambiguously identified in the spectrum. Data presented here were generated using a synthetic template mimicking a portion of the HIV type 1 protease gene. DNA sequencing was performed in one tube by combining the biotinylated ddNTPs, regular dNTPs, DNA polymerase, and reaction buffer (9).

TABLE 1
Cloned enterovirus targets

| Virus | 5′ UTR | pol |
|---|---|---|
| Echovirus 3 | + | + |
| Echovirus 6 | + | + |
| Echovirus 9 | + | + |
| Echovirus 16 | + | + |
| Echovirus 17 | + | + |
| Echovirus 25 | + | + |
| Echovirus 30 | + | + |
| Poliovirus 1 | + | + |
| Poliovirus 2 | + | + |
| Poliovirus 3 | + | + |
| Coxsackie A9 | + | + |
| Coxsackie B2 | + | + |

In Propagation

Coxsackie (A9), Coxsackie A16, Coxsackie B1, Coxsackie B3,
Coxsackie B4, Coxsackie B5, Coxsackie B6, Echovirus 7,
Echovirus 13, Echovirus 18

Cloning Viral Targets as Controls for Mass-Tag PCR

Multiple sequence alignment algorithms have been used by our bioinformatics core to extract the most conserved genomic regions amongst the GenBank published enteroviral sequences. Regions wherein sequence conservation meets or exceeds 80% for an enteroviral serogroup or genetically related subgroup have been identified in the 5′-untranslated region (UTR) and the polymerase gene (3D) of the enterovirus genus. A representative collection of virus isolates has been obtained to generate calibrated standards for Mass-Tag PCR (Table 1). The current panel includes 22 isolates representing all characterized serogroups of pathogenic relevance (A, B, C, and D; covering about 90% of all US enterovirus isolates in the past 10 years; the remaining 10% include non-typed isolates). Twelve isolates have been grown and the relevant regions cloned for spotting onto DNA microarrays and use as transcript controls for DSDD, multiplex bead based, and real time PCR assays. Viruses can be propagated in the appropriate cell lines to generate working and library stocks (Rd, Vero, HeLa, Fibroblast, or WI-38 cells). Library stocks can be frozen and maintained in curated collections at −70° C. Viral RNA can be extracted from working stocks using Tri-Reagent (Molecular Research Center, Inc.). Purified RNA can be reverse transcribed into cDNA using random hexamer priming [to avoid 3′ bias] (Superscript II, Invitrogen/Life Technologies).

Target regions of 100-200 bp representing the identified core sequences will be amplified by PCR from cDNA template using virus-specific primers. Products are cloned (via a single deoxyadenosine residue added in template-independent fashion by common Taq-polymerases to 3′-ends of amplification products) into the transcription vector pGEM T-Easy (Promega Corp.). After transformation and amplification in Escherichia coli, plasmids are analyzed by restriction mapping and automated dideoxy sequencing (Columbia Genome Center) to determine insert orientation and fidelity of PCR. Plasmid libraries will be maintained as both cDNAs and glycerol stocks.

Multiple sequence alignment algorithms can be used to identify highly conserved (>95%) sequence stretches of 20-30 bp length within the identified core sequences to serve as targets for primer design.

Synthesis of Primers for Use in Mass-Tag PCR

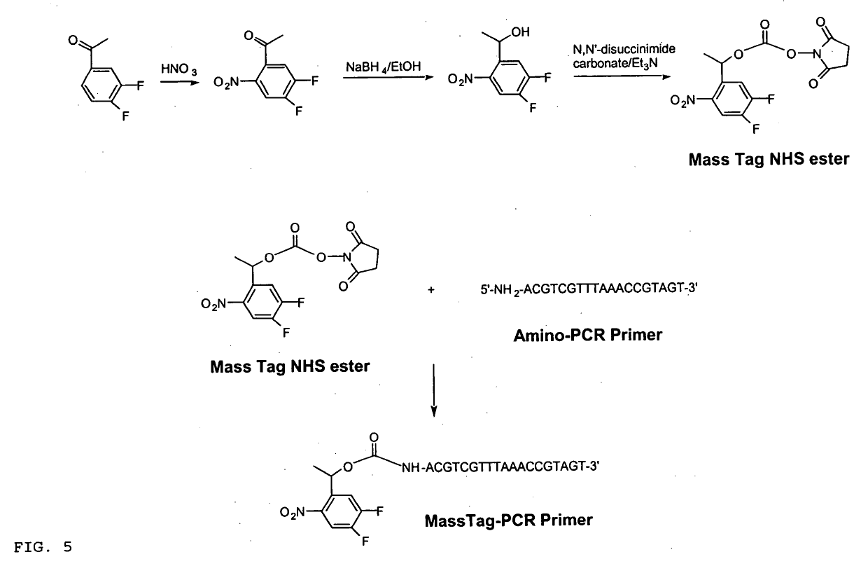

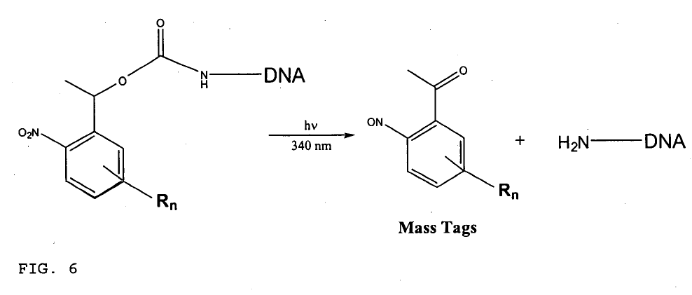

Highly conserved target regions within the core sequences suitable for primer design are identified by using multiple sequence alignment algorithms adjusted for the appropriate window size (20-30 bp) and conservation threshold (>95%). Final alignments are color-coded to facilitate manual inspection. Parameters implicated in primer performance including melting temperature, 3′-terminal stability, internal stability, and propensity of potential primers to form stem loops or primer-dimers can be assessed using standard primer selection software programs OLIGO (Molecular Biology Insights), Primer Express (PE Applied Biosystems), and Primer Premiere (Premiere Biosoft International). Primers can be synthesized with a primary amine-group at the 5′-end for subsequent coupling to NHS esters of the mass tags (FIG. 5). Mass tags with molecular weights between 150 and 2500 daltons can be generated by introducing various functional groups [Rn] in the mass tag parent structure to code for individual primers and thus for the targeted viral sequence (see FIG. 6; also showing the photocleavage reaction). MS is capable of detecting small stable molecules with high sensitivity, a mass resolution greater than one dalton, and the detection requires only microseconds. The mass tagging approach has been successfully used to detect multiplex single nucleotide polymorphisms (15).

Sensitivity and Specificity of Mass-Tag PCR for Detection of Enteroviral Transcripts

Although the method disclosed here is useful for detecting viral RNA, plasmid DNA is an inexpensive, easily quantitated sequence target; thus, primer sets can be initially validated by using dilutions of linearized plasmid DNA. Plasmids are selected to carry the viral insert in mRNA sense orientation with respect to the T7 promoter sequence. Plasmids will be linearized by restriction digestion using an appropriate enzyme that cleaves in the polylinker region downstream of the insert. Where the cloned target sequence is predicted to contain the available restriction sites, a suitable unique restriction site is introduced via the PCR primer used during cloning of the respective target. Purified linearized plasmid DNA is serially diluted in background DNA (human placenta DNA, Sigma) to result in 5×10⁵, 5×10⁴, 5×10³, 5×10², 5×10¹, and 5×10⁰ copies per assay.

Once optimal primer sets for detection of all relevant enteroviruses are identified, the sensitivity of the entire procedure including RNA extraction and reverse transcription is assessed. Synthetic RNA transcripts of each target sequence are generated from the linearized plasmid DNA using T7 RNA polymerase. Transcripts are serially diluted in background RNA relevant to the primary hypothesis (e.g., ALS, normal spinal cord RNA). Individual dilutions representing 5×10⁵, 5×10⁴, 5×10³, 5×10², 5×10¹, and 5×10⁰ copies per assay in a background of 25 ng/ul total RNA are extracted with Tri-Reagent, reverse transcribed, and then subjected to Mass-Tag PCR.

Specificity of the identified primer sets relevant to multiplexing can be assessed by using one desired primer set in conjunction with its respective target sequence at 5 times threshold concentration in the presence of all other, potentially cross-reacting, target sequences at a 10²-, 10⁴- and 10⁶-fold excess.

PCR amplification is performed using photocleavable mass tagged primers in the presence of a biotinylated nucleotide (e.g. Biotin-16-dUTP, Roche) to allow removal of excess primer after PCR. Amplification products will be purified from excess primer by binding to a streptavidin-coated solid phase such as streptavidin-Sepharose (Pharmacia) or streptavidin coated magnetic beads (Dynal) via biotin-streptavidin interaction.

Molecular mass tags can be made cleavable by irradiation with near UV light (˜350 nm), and the released tags introduced by either chromatography or flow injection into a pneumatic nebulizer for detection in an atmospheric pressure chemical ionization mass spectrometer. Alternatively, to increase the specificity of detection by analyzing only PCR products of the expected size range, the mass tagged amplicons, can be size-selected (without the requirement for biotinylated nucleotides) using HPLC.

Multiplex Detection and Identification of Enteroviral Transcripts

A method that allows simultaneous detection of a broad range of enteroviruses with similar sensitivity was developed. A series of 4 primer sets were identified in the 5′-UTR predicted to detect all enteroviruses. These can be combined into two or perhaps even one mixed set for multiplex PCR. Two different genomic regions, 5′-UTR and polymerase, are targeted with independent primer panels, in order to confirm presence of enterovirus infection.

Once the presence of enteroviral sequences are confirmed using broad range primer sets, a different primer set is used to discriminate amongst the various enteroviral species. Whereas broad range primers are be selected from the highly conserved 5′-UTR and polymerase 3D gene regions, the primer sets used to identify the enterovirus species target the most divergent genomic regions in VP3 and VP1.

Limitations must be considered in that although cerebral spinal fluid is unlikely to contain more than a single enterovirus (the virus responsible for clinical disease in an individual patient), individual stool samples may contain several enteroviruses. It is important, therefore, that assays not favor amplification or detection of one viral species over another. Second, multiplexing can result in loss of sensitivity. Thus, panels should be assessed for sensitivity (and specificity) with addition of new primer sets.

Direct MS-Sequencing of PCR Amplified Enteroviral Transcripts for Virus Species Identification

MALDI MS has been explored widely for DNA sequencing; however, this approach requires that the DNA sequencing fragments be free from alkaline and alkaline earth salts, as well as other contaminants, to ensure accurate measurements of the masses of the DNA fragments. We explored a novel MS DNA sequencing method that generates Sanger-sequencing fragments using biotinylated dideoxynucleotides labeled with mass tags.

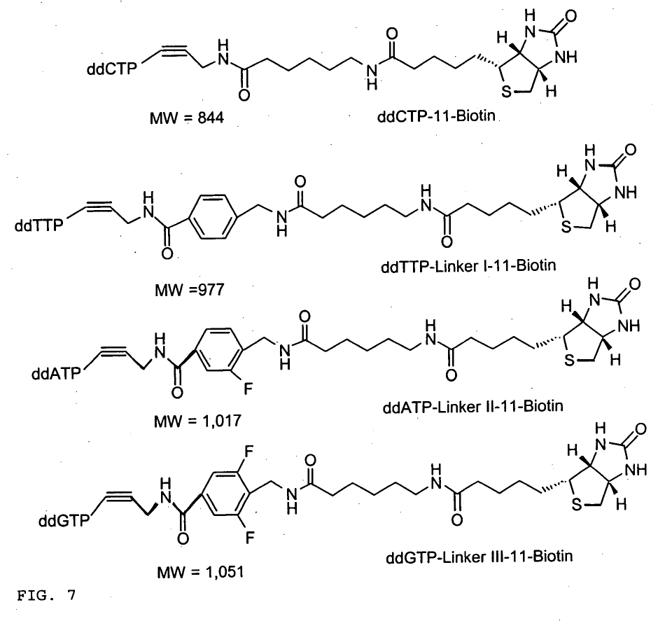

The ability to distinguish various nucleotide bases in DNA using mass spectrometry is dependent on the mass differences of the DNA ladders in the mass spectra. Smith et al. have shown that using dye labeled ddNTP paired with a regular dNTP to space out the mass difference can increase the detection resolution in a single nucleotide extension assay (10). Preliminary studies using biotin-11-dd(A, C, G)TPs and biotin-16-ddTTP, indicated that the smallest mass difference between any two nucleotides is 16 daltons. To enhance the ability to distinguish peaks in the sequencing spectra, the mass separation of the individual ddNTPs can be increased by systematically modifying the biotinylated dideoxynucleotides by incorporating mass linkers assembled using 4-aminomethyl benzoic acid derivatives. The mass linkers can be modified by incorporating one or two fluorine atoms to further space out the mass differences between the nucleotides. The structures of the newly designed biotinylated ddNTPs are shown in FIG. 7. Linkers are attached to the 5 position on the pyrimidine bases (C and T), and to the 7 position on the purines (A and G) to facilitate conjugation with biotin. It has been established that modification of these positions on the bases in the nucleotides, even with bulky energy transfer (ET) fluorescent dyes, still allows efficient incorporation of the modified nucleotides into the DNA strand by DNA polymerase (24, 31). Biotin and the mass linkers are considerably smaller than the ET dyes, ameliorating difficulties in incorporation of ddNTP-linker-biotin molecules into DNA strands in sequencing reactions.

The DNA sequencing fragments that carry a biotin at the 3′-end are made free from salts and other components in the sequencing reaction by capture with streptavidin-coated magnetic beads. Thereafter, the correctly terminated biotinylated DNA fragments are released and loaded onto the mass spectrometer. Results indicate that MS can produce high resolution of DNA-sequencing fragments, fast separation on microsecond time scales, and eliminate the compressions associated with gel electrophoresis.

Amplification products obtained by PCR with broad range 5′-UTR or polymerase 3D primer sets can be used as template. Sequencing permits discrimination between bona fide enteroviral amplification products and artifacts. Where analysis of the semi-divergent sequence region located toward the 3′-end of the 5′-UTR region is inadequate for speciation, targeting the more divergent VP3 and/or VP1 regions is preferred.

### REFERENCES FOR EXAMPLE 1

1. Berger, M. M., N. Kopp, C. Vital, B. Redl, M. Aymard, and B. Lina 2000. Detection and cellular localization of enterovirus RNA sequences in spinal cord of patients with ALS. Neurology. 54:20-25.
2. Briese, T., W. G. Glass, and W. I. Lipkin 2000. Detection of West Nile virus sequences in cerebrospinal fluid. Lancet. 355:1614-1615.
3. Briese, T., X. Y. Jia, C. Huang, L. J. Grady, and W. I. Lipkin 1999. Identification of a Kunjin/West Nile-like flavivirus in brains of patients with New York encephalitis. Lancet. 354:1261-1262.
4. Casas, I., G. F. Palacios, G. Trallero, D. Cisterna, M. C. Freire, and A. Tenorio 2001. Molecular characterization of human enteroviruses in clinical samples: comparison between VP2, VP1, and RNA polymerase regions using RT nested PCR assays and direct sequencing of products J. Med. Virol. 65:138-148.
5. Challoner, P. B., K. T. Smith, J. D. Parker, D. L. MacLeod, S. N. Coulter, T. M. Rose, E. R. Schultz, J. L. Bennett, R. L. Garber, M. Chang, P. A. Schad, P. M. Stewart, R. C. Nowinski, J. P. Brown, and G. C. Burmer 1995. Plaque-associated expression of human herpesvirus 6 in multiple sclerosis. Proc. Natl. Acad. Sci. USA. 92:7440-7444.
6. Chang, Y., E. Cesarman, M. S. Pessin, F. Lee, J. Culpepper, D. M. Knowles, and P. S. Moore 1994. Identification of herpesvirus-like DNA sequences in AIDS-associated Kaposi's sarcoma. Science. 266:1865-1869.
7. Choo, Q. L., G. Kuo, A. J. Weiner, L. R. Overby, D. W. Bradley, and M. Houghton 1989. Isolation of a cDNA clone derived from a blood-borne non-A, non-B viral hepatitis genome. Science. 244:359-362.
8. Diehn, M., and D. A. Relman 2001. Comparing functional genomic datasets: lessons from DNA microarray analyses of host-pathogen interactions. Curr. Opin. Microbiol. 4:95-101.
9. Edwards, J. R., Y. Itagaki, and J. Ju 2001. DNA sequencing using biotinylated dideoxynucleotides and mass spectrometry. Nucleic Acid Res. 29:1-6.
10. Fei, Z., T. Ono, and L. M. Smith 1998. MALDI-TOF mass spectrometric typing of single nucleotide polymorphisms with mass-tagged ddNTPs. Nucleic Acids Res. 26:2827-2828.
11. Fu, D. J., K. Tang, A. Braun, D. Reuter, B. Darnhofer-Demar, D. P. Little, M. J. O'Donnell, C. R. Cantor, and H. Koster 1998. Sequencing exons 5 to 8 of the p53 gene by MALDI-TOF mass spectrometry. Nat. Biotechnol. 16:381-384.
12. Hubank, M., and D. G. Schatz 1994. Identifying differences in mRNA expression by representational difference analysis of cDNA. Nucleic Acids Res. 22:5640-5648.
13. Ju, J. 1999. Nucleic Acid Sequencing with Solid Phase Capturable Terminators. U.S. Pat. No. 5,876,936.
14. Ju, J., and K. Konrad 2000. Nucleic Acid Sequencing with Solid Phase Capturable Terminators Comprising a Cleavable Linking Group. U.S. Pat. No. 6,046,005.
15. Kokoris, M., K. Dix, K. Moynihan, J. Mathis, B. Erwin, P. Grass, B. Hines, and A. Duesterhoeft 2000. High-throughput SNP genotyping with the Masscode system. Mol. Diagn. 5:329-340.
16. Liang, P., and A. B. Pardee 1992. Differential display of eukaryotic messenger RNA by means of the polymerase chain reaction. Science. 257:967-971.
17. Lipkin, W. I., G. H. Travis, K. M. Carbone, and M. C. Wilson 1990. Isolation and characterization of Borna disease agent cDNA clones. Proc. Natl. Acad. Sci. USA. 87:4184-4188.
18. Lisitsyn, N., N. Lisitsyn, and M. Wigler 1993. Cloning the differences between two complex genomes. Science. 259:946-951.
19. Nichol, S. T., C. F. Spiropoulou, S. Morzunov, P. E. Rollin, T. G. Ksiazek, H. Feldmann, A. Sanchez, J. Childs, S. Zaki, and C. J. Peters 1993. Genetic identification of a hantavirus associated with an outbreak of acute respiratory illness. Science. 262:914-917.
20. Palacios, G., I. Casas, A. Tenorio, and C. Freire 2002. Molecular identification of enterovirus by analyzing a partial VP1 genomic region with different methods J. Cin. Microbiol. 40:182-192.
21. Relman, D. A. 1999. The search for unrecognized pathogens. Science. 284:1308-1310.
22. Relman, D. A., J. S. Loutit, T. M. Schmidt, S. Falkow, and L. S. Tompkins 1990. The agent of bacillary angiomatosis. An approach to the identification of uncultured pathogens. N. Engl. J. Med. 323:1573-1580.
23. Relman, D. A., T. M. Schmidt, R. P. MacDermott, and S. Falkow 1992. Identification of the uncultured bacillus of Whipple's disease. N. Engl. J. Med. 327:293-301.
24. Rosenblum, B. B., L. G. Lee, S. L. Spurgeon, S. H. Khan, S. M. Menchen, C. R. Heiner, and S. M. Chen 1997. New dye-labeled terminators for improved DNA sequencing patterns. Nucleic Acids Res. 25:4500-4504.
25. Sanger, F., S. Nickeln, and A. R. Coulson 1977. DNA sequencing with chain-terminating inhibitors Proc Natl Acad Sci USA. 74:5463-5467.
26. Taylor, L. A., C. M. Carthy, D. Yang, K. Saad, D. Wong, G. Schreiner, L. W. Stanton, and B. M. McManus 2000. Host gene regulation during coxsackievirus B3 infection in mice: assessment by microarrays. Circ. Res. 87:328-334.
27. VandeWoude, S., J. A. Richt, M. C. Zink, R. Rott, O. Narayan, and J. E. Clements 1990. A Borna Virus cDNA Encoding a Protein Recognized by Antibodies in Humans with Behavioral Diseases. Science. 250:1278-1281.
28. Walker, M. P., R. Schlaberg, A. P. Hays, R. Bowser, and W. I. LIpkin 2001. Absence of echovirus sequences in brain and spinal cord of amyotrophic lateral sclerosis patients. Annals Neurol. 49:249-253.
29. Wu, K. J., A. Steding, and C. H. Becker 1993. Matrix-assisted laser desorption time-of-flight mass spectrometry of oligonucleotides using 3-hydroxypicolinic acid as an ultraviolet-sensitive matrix. Rapid Commun. Mass Spectrom. 7:142-146.
30. Zhu, H., J. P. Cong, G. Mamtora, T. Gingeras, and T. Shenk 1998. Cellular gene expression altered by human cytomegalovirus: global monitoring with oligonucleotide arrays. Proc. Natl. Acad. Sci. USA. 95:14470-14475.
31. Zhu, Z., J. Chao, H. Yu, and A. S. Waggoner 1994. Directly labeled DNA probes using fluorescent nucleotides with different length linkers. Nucleic acids Res. 22:3418-3422.

### Example 2

### Multiplex Mass Tag PCR Detection of Respiratory Pathogens

Background and Significance

The advent of SARS in 2003 poignantly demonstrated the urgency of establishing rapid, sensitive, specific, inexpensive tools for differential laboratory diagnosis of infectious diseases. Through unprecedented global collaborative efforts, the causative agent was rapidly implicated and characterized, facilitating development of serologic and molecular assays for infection, and containment of the outbreak. Nonetheless, as the northern hemisphere entered the winter season of 2004, the diagnosis of SARS still rested on clinical and epidemiological as well as laboratory criteria.

Methods for cloning nucleic acids of microbial pathogens directly from clinical specimens offer new opportunities to investigate microbial associations in diseases. The power of these methods is not only sensitivity and speed but also the potential to succeed where methods for pathogen identification through serology or cultivation may fail due to absence of specific reagents or fastidious requirements for agent replication.

Various methods are employed or proposed for cultivation-independent characterization of infectious agents. These can be broadly segregated into methods based on direct analysis of microbial nucleic acid sequences, direct analysis of microbial protein sequences, immunological systems for microbe detection, and host response profiling. Any comprehensive armamentarium should include most, if not all, of these tools. Nonetheless, classical methods for microbiology remain important. Indeed, the critical breakthrough during the SARS outbreak was the cultivation of the agent in tissue culture.

Real-time PCR methods have significantly changed diagnostic molecular microbiology by providing rapid, sensitive, specific tools for detecting and quantitating genetic targets. Because closed systems are employed, real-time PCR is less likely than nested PCR to be confounded by assay contamination due to inadvertent aerosol introduction of amplicon/positive control/cDNA templates that can accumulate in diagnostic laboratories. The specificity of real time PCR is both a strength and a limitation. Although the potential for false positive signal is low so is the utility of the method for screening to detect related but not identical genetic targets. Specificity in real-time PCR is provided by two primers (each approximately 20 matching nucleotides (nt) in length) combined with a specific reporter probe of about 27 nt. The constraints of achieving hybridization at all three sites may confound detection of diverse, rapidly evolving microbial genomes such as those of single-stranded RNA viruses. These constraints can be compensated in part by increasing numbers of primer sets accommodating various templates. However, because real-time PCR relies on fluorescent reporter dyes, the capacity for multiplexing is limited to the number of emission peaks that can be unequivocally separated. At present up to four dyes can be identified simultaneously. Although the repertoire may increase, it will unlikely to change dramatically.

To address the need for enhanced multiplex capacity in diagnostic molecular microbiology we have established a PCR platform based on mass tag reporters that are easily distinguished in MS as discrete signal peaks. Major advantages of the PCR/MS system include: (1) hybridization to only two sites is required (forward and reverse primer binding sites) vs real time PCR where an intermediate third oligonucleotide is used (probe binding site); this enhances flexibility in primer design; (2) tried and proven consensus PCR primers can be adapted to PCR/MS; this reduces the time and resources that must be invested to create new reagents and assay controls; (3) the large repertoire of tags allows highly multiplexed assays; additional tags can be easily synthesized to allow further complexity; and (4) sensitivity of real time PCR is maintained. We view PCR/MS as a tool with which to rapidly screen clinical materials for the presence of candidate pathogens. Thereafter, targeted secondary tests, including real time PCR, can be used to quantitate microbe burden and pursue epidemiologic studies.

Preliminary Data

We have developed bioinformatic tools to facilitate sequence alignments, motif identification, and primer design; established banks of viral strains, cDNA templates, and primers; and built relationships with collaborators in national and global public health laboratory networks that provide access to data, organisms, sera, and cDNAs that facilitate assay development and validation. Over the past two years we have integrated PCR and MS into a stable and sensitive digital assay platform similar in sensitivity and efficiency to real time PCR but with the advantages of simultaneous detection and discrimination of multiple targets. Using the 4 tags created for DNA sequencing we initially tested the method with flavivirus and bunyavirus targets as a proof of principle for an encephalitis project. The collaboration was later expanded to include two industrial partners: QIAGEN GmbH, a partner with a large validated library of proprietary photocleavable mass tags (Masscode™) and expertise in manufacture and commercial distribution, and Griffin Analytical Technologies, a partner actively engaged in design and fabrication of low cost portable MS instruments for field applications.

Selection of APCI LCMS Platform

Mass spectrometry is a rapid, sensitive method for detection of small molecules. With the development of Ionization techniques such as matrix assisted laser desorption ionization (MALDI) and electrospray ionization (ESI), MS has become a indispensable tool in many areas of biomedical research. Although these ionization methods are suitable for the analysis of bioorganic molecules, such as peptides and proteins, improvements in both detection and sample preparation will be required before mass spectrometry can be used to directly detect long DNA fragments. A major confound in exploiting MS for genetic investigation has been that long DNA molecules are fragmented during the analytic process. The mass tag approach we have developed overcomes this limitation by detecting small stable mass tags that serve as signatures for specific DNA sequences rather than the DNA sequences themselves.

We have explored the kinetics of photocleavable primer conjugation. Ionization and detection of the photocleaved mass tags have been extensively characterized using atmospheric pressure chemical ionization (APCI) as the ionization source while using a single quadrupole mass spectrometer as the detector (Jingyue et al., Kim et al. 2003; Kokoris et al. 2000). Because buffer and inorganic salts impact ionization efficiency, performance in ESI was determined to be critically dependent upon sample preparation conditions. In MALDI, matrix must be added prior to sample introduction into the mass spectrometer, which is a time consuming step that requires costly sample spotting instrumentation. Similary, speed is often limited by the need to search for an ideal irradiation spot to obtain interpretable mass spectra.

In contrast, APCI is much more tolerant of residual inorganic salts (than ESI) and does not require mixing with matrix to prepare crystals on a target plate. Thus, mass tag solutions can be injected directly into the MS via a Liquid Chromatography (LC) delivery system. Since mass tags ionize well under APCI conditions and have small mass values (less that 800 amu), they are detected with high sensitivity (<5 femtomolar limit of detection) with the APCI-Quadrupole LCMS platform.

Methods for synthesis and APCI-MS analysis of mass tags coupled to DNA fragments are illustrated in FIG. 8 where precursors are (a) acetophenone; (b) 4-fluoroacetophenone; (c) 3-methoxyacetophenone; and (d) 3,4-dimethoxyacetophenone.

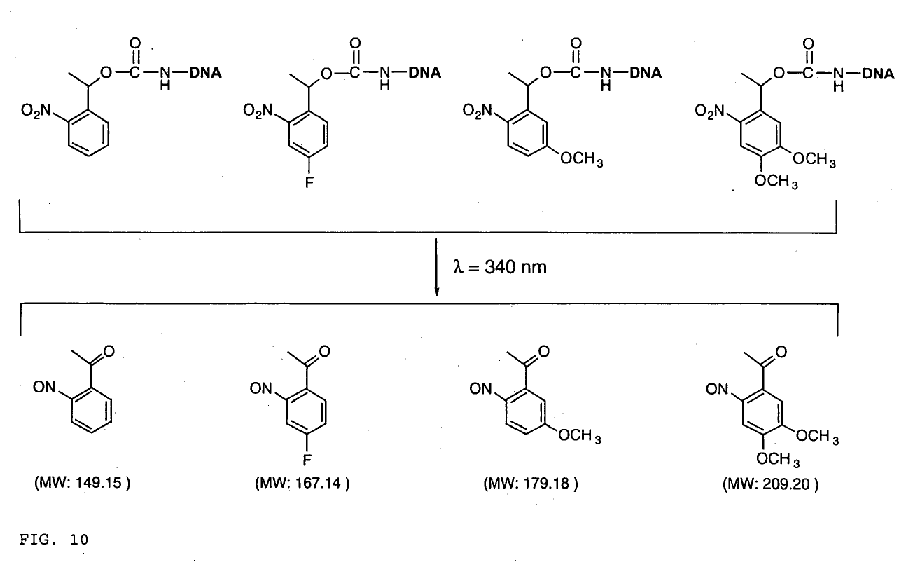

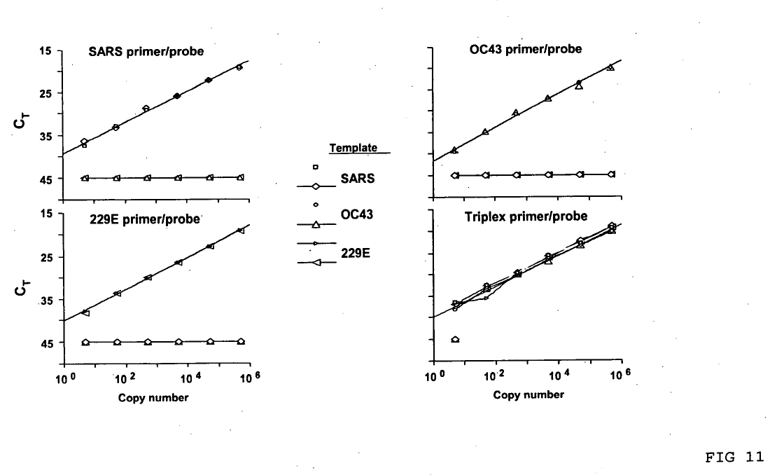

Upon nitration and reduction, the photoactive tags are produced and used to code for the identity of different primer pairs. An example for photocleavage and detection of four tags is shown in FIG. 9 which shows APCI mass spectra for four mass tags after from the corresponding primers (mass tag # 1,2-nitrosoacetophenone, m/z 150; mass tag # 2,4-fluoro-2-nitrosoacetophenone, m/z 168; mass tag # 3,5-methoxy-2-nitrosoacetophenone, m/z 180; mass tag # 4,4,5-dimethoxy-2-nitrosoacetopheone, m/z 210). The four mass tag-labeled primers were mixed together and the mixture was irradiated under UV light (λ˜340 nm) for 5 seconds, introduced into an APCI mass spectrometer and analyzed for the four masses to produce the above spectrum. The peak with m/z of 150 is mass-tag 1, 168 is mass-tag 2, 180 is mass-tag 3 and 210 is mass-tag 4. The mechanism for release of these tags from DNA is shown in FIG. 10—Four mass tag-labeled DNA molecules (Bottom) Chemical structures of the corresponding photocleaved mass tags (2-nitrosoacetophenone, 4-fluoro-2-nitrosoacetophenone, 5-methoxy-2-nitrosoacetophenone and 4,5-dimethoxy-2-nitrosoacetophenone) after UV irradiation at 340 nm. This result indicates that the 4 compounds designed as mass tags are stable and produce discrete high-resolution digital data in an APCI mass spectrometer. The unique m/z from each mass tag translates to the identity of a viral sequence. In a recent collaboration with Qiagen, which has used a library of mass tags to discriminate up to 25 SNPs (Kokoris et al. 2000), we have significantly expanded the number of the mass tags.

Establishment of a PCR/MS Assay for Respiratory Pathogens

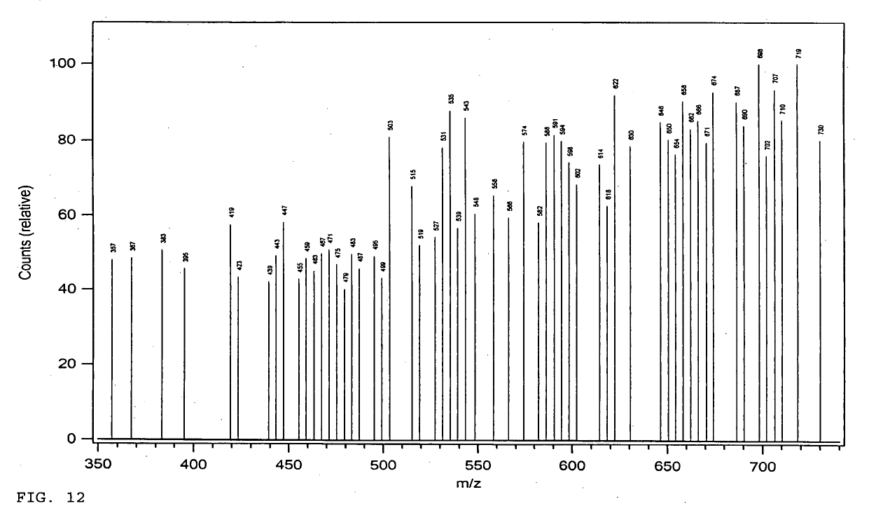

During the SARS 2003 Beijing outbreak we established a specific and sensitive real time PCR assay for SARS-CoV (Zhai et al, 2004). The assay was extended to allow simultaneous detection of SARS-CoV as well as human coronaviruses OC43 and 229E in light of recent data from China suggesting the potential for coinfection and increased morbidity (FIG. 11). This human coronavirus assay (3 viral genes and 1 housekeeping gene) exhausted the repertoire of fluorescent tags with which to pursue multiplex real time PCR analysis of clinical materials. The importance of extending rapid molecular assays to include other respiratory pathogens is reinforced by the reappearance of SARS in China and reports of a new highly virulent influenza virus strain in Vietnam.

To build a more comprehensive respiratory pathogen surveillance assay we adapted the human coronavirus primers to the PCR/MS platform, and added reagents required to detect other relevant microbes. Influenza A virus was included through a set of established primer sequences obtained through Georg Pauli (Robert Koch Institute, Germany; Schwaiger et al 2000). For the bacterial pathogen M. pneumoniae we also used unmodified primer sequences published for real time PCR (Welti et al 2003) to evaluate their use on the PCR/MS platform. Using a panel of mass tags developed by QIAGEN, experiments were performed demonstrating the feasibility of detecting several respiratory pathogens in a single multiplexed assay on the PCR/MS platform.

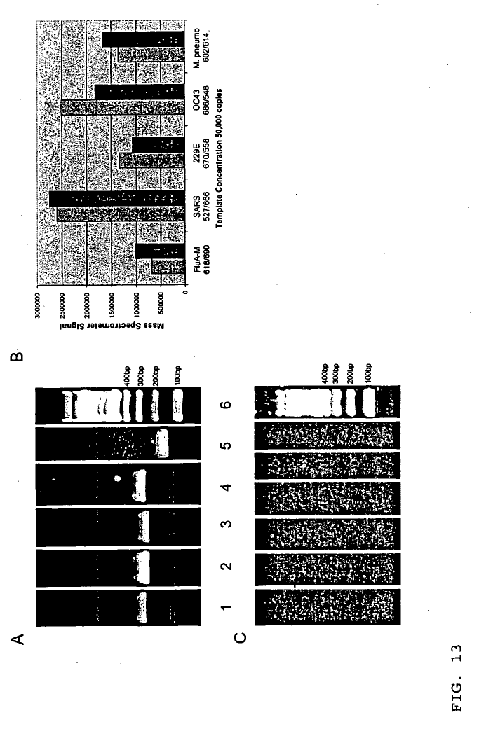

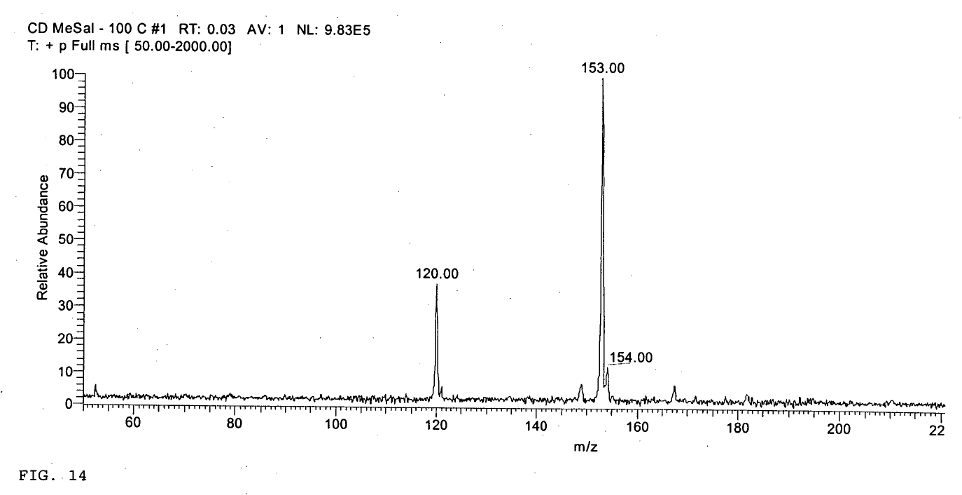

The current Masscode™ photocleavable mass tag repertoire comprises over 80 tags. FIG. 12 demonstrates the specificity of the mass tag detection approach in an example where 58 different mass tags conjugated to oligonucleotides via a photocleavable linkage were identified after UV cleavage and MS. Each of the 10 primers for the 5-plex assay (SARS-CoV, CoV-229E, CoV-OC43, Influenza A virus, and M. pneumoniae) was conjugated to a different mass tag such that the identity of a given pathogen was encoded by a specific binary signal (e.g. SARS-CoV, forward primer, 527 amu; reverse primer 666 amu; see FIG. 13B).

The presence of mass tags did not impair performance of primers in PCR and yielded clear signals for all 5 agents (FIG. 13A, 13B—Singleplex mass tag PCR for (1) Influenza A virus matrix protein (618 amu fwd-primer, 690 amu rev-primer), human coronaviruses (2) SARS (527/666), (3) 229E (670/558), (4) OC43 (686/548), and the bacterial agent (5) M. pneumoniae (602/614). (6) 100 bp ladder). No noise was observed using unmodified or mass tag-modified primer sets in a background of 125 ng of normal total human DNA per assay (FIG. 13C). In subsequent experiments we extended the respiratory pathogen panel to include respiratory syncytial virus groups A and B. Non-optimized pilot studies in this 7-plex system indicated a detection threshold of <500 molecules. As a test of feasibility for PCR/MS detection of coinfection, mixtures of DNA templates representing two different pathogens were analyzed successful detection of two targets confirmed the suitability of this technology for clinical applications where coinfection may be critical to pathogenesis and epidemiology.

Establishment of a Platform for Portable MS

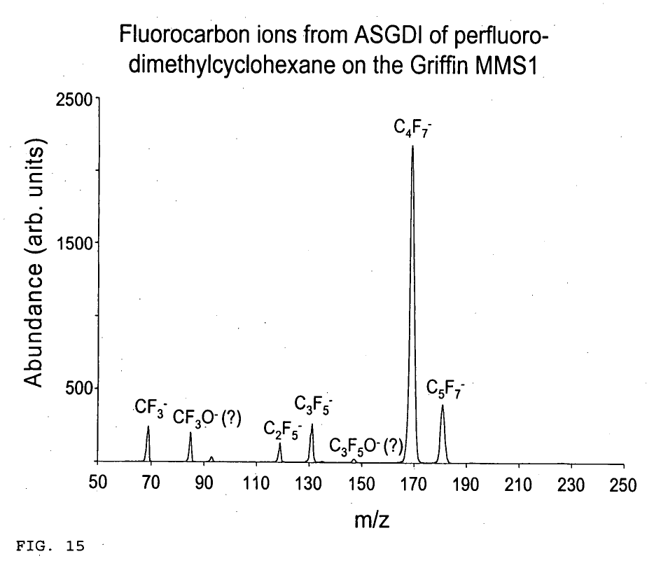

Griffin has developed a portable mass spectrometer that is roughly the size of a tower computer (including vacuum system), weighs less than 50 lbs, and consumes ˜150 W depending on operating conditions. This system has a mass range of 400 Da with unit mass resolution. It has been used to detect part-per-trillion level atmospheric constituents. FIG. 14 shows a representative spectrum of methyl salicylate collected on a miniature cylindrical ion trap mass analyzer coupled to a corona discharge ionization source (data collected in Prof. R. G. Cooks research laboratory at Purdue University). This data demonstrates the feasibility of using this type of instrumentation to detect the mass tags of interest as well as the specificity of the ionization source. FIG. 14 shows mass spectrum representative of data collected using a miniature cylindrical ion trap mass analyzer coupled with a corona discharge ionization source.

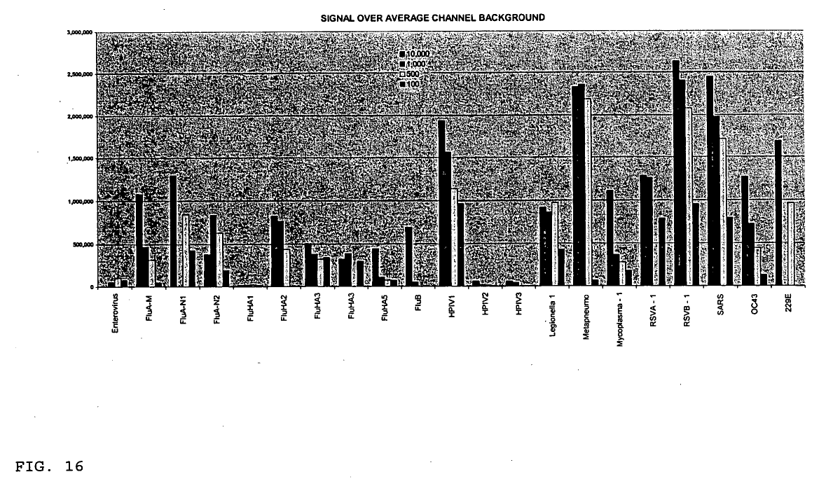

FIG. 15 shows a mass spectrum of perflouro-dimethclcyclohexane collected on a prototype atmospheric sampling glow discharge ionization (ASGDI) source. ASGDI is an external ionization source related to the APCI source discussed here.

Experimental Design

Labeled amplification products are generated during PCR amplification with mass tagged primers. After isolation from non-incorporated primers by binding to silica in Qiagen 96-well or 384-well PCR purification modules, products are eluted into the injection module of the mass-spectrometer. The products traverse the path of a UV light source prior to entering the nebulizer, releasing photocleavable tags. (one each from the forward and reverse primer). Mass tags are then ionized. Analysis of the mass code spectrum defines the pathogen composition of the specimen.

A non-comprehensive list of target pathogens is listed in Tables 2 and 3. Forward and reverse primer pairs for pathogens listed in Table 2 are (reading from top to bottom starting with RSV-A and ending with M. Pneumoniae), SEQ ID NOS:1 and 2, 3 and 4, 9 and 10, 21 and 22, 23 and 24, 26 and 27, and 49 and 50.

TABLE 2

Respiratory Panel Mass-Tag Primers

| | Forward | | Reverse | |
|---|---|---|---|---|
| Pathogen | primer | Sequence | primer | Sequence |
| RSV A | RSA- | AgATCAACTTCTgTC | RSV- | gCACATCATAATTAggAg |
| | U1137 | ATCCAgCAA | L1192 | TATCAAT |
| RSV B | RSB- | AAgATgCAAATCAT | RSV-1318 | TgATATCCAgCATCTTTA |
| | U1248 | AAATTCACAggA | | AgTATCTTTATAgTg |
| Influenza A | | | | |
| (N1) | | | | |
| Influenza A | | | | |
| (N2) | | | | |
| Influenza A | AM-U151 | CATggAATggCTAAA | AM-L397 | AAgTgCACCAgCAgAATA |
| (M) | | gACAAgACC | | ACTgAg |
| Influenza B | | | | |
| SARS-CoV | CIID- | AAg CCT CgC CAA | CIID- | AAg TCA gCC ATg TTC |
| | 28891F | AAA CgT AC | 29100R | CCg AA |
| 229E-CoV | Taq-Co22- | ggC gCA AgA ATT | Taq-Co22- | TAA gAg CCg CAg CAA |
| | 418F | CAg AAC CA | 636R | CTg C |
| OC43-CoV | Taq-Co43- | TgT gCC TAT TgC | Taq-Co43- | CCC gAT CgA CAA TgT |
| | 270F | ACC Agg AgT | 508R | CAg C |
| Metapneumo- | | | | |
| virus | | | | |
| Parainfluenza | | | | |
| 1 | | | | |
| Parainfluenza | | | | |
| 2 | | | | |
| Parainfluenza | | | | |
| 3 | | | | |
| Parainfluenza | | | | |
| 4 | | | | |
| M | MTPM1 | CCAACCAAACAACA | MTPM2 | ACCTTgACTggAggCCgTT |
| pneumoniae | | ACgTTCA | | A |
| L. | | | | |
| pneumophilae | | | | |
| C. | | | | |
| pneumoniae | | | | |

Design and Synthesis of Primers

Primers are designed using the same approach as employed for the 7-plex assay. Available sequences are be extracted from GenBank. Conserved regions suitable for primer design are identified using standard software programs as well as custom software (patent application XYZ). Primer properties can be assessed by commercial primer selection software including OLIGO (Molecular Biology Insights), Primer Express (PE Applied Biosystems), and Primer Premiere (Premiere Biosoft International). Primers are evaluated for signal strength and specificity against a background of total human DNA.

Isolation and Cloning of Template Standards

Targeted genes can be cloned into the transcription vector pGEM-Teasy (Invitrogen) by conventional RT-PCR cloning methods. Quantitated plasmid standards are used in initial assay establishment. Thereafter, RNA transcripts generated by in vitro transcription, quantitated and diluted in a background of random human RNA (representing brain, liver, spleen, lung and placenta in equal proportions) are employed to establish sensitivity and specificity parameters of RT-PCR/MS assays. One representative isolate for each targeted pathogen/gene is used during initial establishment of the assay.

Inherent in the exquisite sensitivity of PCR is the risk of false positive results due to inadvertent introduction of synthetic templates such as those comprising positive control and calibration reagents, and so calibration reagents are preferred components of kits. Thus, to allow recognition of control vs authentic, natural amplification products, calibration reagents are modified by introducing a restriction enzyme cleavage site in between the primer binding sites through site directed mutagenesis. This approach has been employed in projects concerned with epidemiology of viral infection in various chronic diseases including Bornaviruses in neuropsychiatric disease (NIH/MH57467), measles virus in autism (CDC/American Academy of Pediatrics), and enteroviruses in type I diabetes mellitus (NIH/AI55466).

Multiplex Assay Using Cloned Template Standards

Initially, the performancance of individual primer sets with unmodified primers is tested. Amplification products in these single assays can be detected by gel electrophoresis. This strategy will not serve for multiplex assays because products of individual primer sets will be similar in size i.e. <300 bp. Thus, after confirmation of performance in single assays, mass tagged primers are generated for multiplex analyses. All assays are first optimized for PCR using serial dilutions of plasmid DNA, and then for RT-PCR using serial dilutions of synthetic transcripts. A multiplex assay is considered successful if it detects all target sequences at a sensitivity of 50 copies plasmid DNA per assay and 100 copies RNA per assay. Successful multiplex assay performance includes detection of all permutative combinations of two agents to ensure the feasibility of diagnosing simultaneous infection.

Optimizing Multiplex Assay Using Cell Culture Extracts

After establishing performance parameters with calibrated synthetic reagents, cell culture extracts of authentic pathogens are used. Performance of assays with RNA extracted using readily available commercial systems that do or do not include organic solvents (e.g, Tri-Reagent vs RNeasy) is assessed. A protocol disclosed here employs Tri-Reagent. Similarly, although Superscript reverse transcriptase (Invitrogen) and HotStart polymerase (QIAGEN) can be used, performance of ThermoScript RT (Invitrogen) at elevated temperature can be assessed, as are single-step RT-PCR systems like the Access Kit (Promega). To optimize efficiency where clinical material mass is limited and to reduce the complexity of sample preparation, both viral and bacterial agents can be identified using RT-PCR. Where an agent is characterized by substantive phylogenetic diversity, cell culture systems should include at least three divergent isolates of each pathogen

Sample Processing

Samples may be obtained by nasal swabs, sputum and lavage specimens will be spiked with culture material to optimize recovery methods for viral as well as bacterial agents.

Portable APCI MS Instruments to Support Multiplex PCR/MS Platform

The multiplex mass tag approach is well-suited to implementation on a miniaturized MS system, as the photocleavable mass tags are all relatively low in molecular weight (<500 Da.), and hence the constraints on the mass spectrometer in terms of mass range and mass resolution are not high. The technical challenge associated with this approach is the development of an atmospheric-pressure chemical ionization (APCI) source for use on a miniaturized MS to generate the mass tag ions. Such a source has been coupled with a miniaturized MS in an academic setting.

Detection of NIAD Category A, B, and C Priority Agents

Using the same approach as outlined for respiratory pathogen detection, a multiplex assay for detection of selected NIAD Category A, B, and C priority agents can be created (Table 3). Primers and PCR conditions for several agents are already established and can be adapted to the PCR/MS platform.

TABLE 3
NIAD Priority Agents

B. anthracis
Dengue viruses
West Nile virus
Japanese encephalitis virus
St. Louis encephalitis virus
Yellow Fever virus
La Crosse virus
California encephalitis virus
Rift Valley Fever virus
CCHF virus
VEE virus
EEE virus
WEE virus
Ebola virus
Marburg virus
LCMV
Junin virus
Machupo virus
Variola virus

### Example 3

Background

Efficient laboratory diagnosis of infectious diseases is increasingly important to clinical management and public health. Methods for direct detection of nucleic acids of microbial pathogens in clinical specimens are rapid, sensitive and may succeed where fastidious requirements for agent replication confound cultivation. Nucleic acid amplification systems are indispensable tools in HIV and HCV diagnosis, and are increasingly applied to pathogen typing, surveillance, and diagnosis of acute infectious disease. Clinical syndromes are only infrequently specific for single pathogens; thus, assays for simultaneous consideration of multiple agents are needed. Current multiplex assays employ gel-based formats where products are distinguished by size, fluorescent reporter dyes that vary in color, or secondary enzyme hybridization assays. Gel-based assays are reported that detect 2-8 different targets with sensitivities of 2-100 pfu or <1-5 pfu, depending on whether amplification is carried out in a single or nested format, respectively (Ellis and Zambon 2002, Coiras et all. 2004). Fluorescence reporter systems achieve quantitative detection with sensitivity similar to nested amplification; however, their capacity to simultaneously query multiple targets is limited to the number of fluorescent emission peaks that can be unequivocally separated. At present up to four fluorescent reporter dyes are detected simultaneously (Vet et al. 1999, Verweij et al. 2004). Multiplex detection of up to 9 pathogens was achieved in hybridization enzyme systems; however, the method requires cumbersome post-amplification processing (Gröndahl et al. 1999).

To address the need for sensitive multiplex assays in diagnostic molecular microbiology we created a polymerase chain reaction (PCR) platform wherein microbial gene targets are coded by 64 distinct mass tags. Here we describe this system, mass tag PCR, and demonstrate its utility in differential diagnosis of respiratory tract infections.

Oligonucleotide primers for mass tag PCR were designed to detect the broadest number of members for a given pathogen species through efficient amplification of a 50-300 basepair product. In some instances we selected established primer sets; in others we employed a software program designed to cull sequence information from GenBank, perform multiple alignments, and maximize multiplex performance by selecting primers with uniform melting temperatures and minimal cross-hybridization potential. Primers, synthesized with a 5′ C6-spacer and aminohexyl modification, were covalently conjugated via a photocleavable linkage to small molecular weight tags (Kokoris et al. 2000) to encode their respective microbial gene targets. Forward and reverse primers were labeled with differently sized tags to produce a dual code for each target that facilitates assessment of signal specificity.

Microbial gene target standards for sensitivity and specificity assessment were cloned by PCR using cDNA template obtained by reverse transcription of extracts from infected cultured cells or by assembly of overlapping synthetic polynucleotides. Cloned standards representing genetic sequence of the targeted microbial pathogens were diluted in 12.5 ug/ml human placenta DNA (Sigma, St. Louis, Mo., USA) and subjected to multiplex PCR amplification using the following cycling protocol: 9×C for X sec., 55 C for X sec., 72 C for X sec.; 50 cycles, MJ PTC200 (MJ Research, Waltham, Mass., USA). Amplification products were purified using QIAquick 96 PCR purification cartridges (Qiagen, Hilden, Germany) with modified binding and wash buffers (RECIPES). Mass tags of the amplified products were analyzed after ultraviolet photolysis and positive-mode atmospheric pressure chemical ionization (APCI) by single quadrapole mass spectrometry. FIG. 1 indicates discrimination of individual microbial targets in a 21-plex assay comprising sequences of 16 human pathogens. The threshold of detection met or exceeded 500 molecules corresponding in sensitivity to less than 0.1 TCID₅₀/ml (0.001 TCID₅₀/assay), in titered cell culture virus of coronaviruses as well as parainfluenza viruses (data not shown). For 19 of 21 microbial targets the detection threshold was less than 100 molecules (Table 4).

We next analyzed samples from individuals with respiratory infection using a larger panel comprising 30 gene targets (26 pathogens). Mass Tag PCR correctly identified infection with respiratory syncitial, human parainfluenza, SARS corona, adeno, entero, metapneumo and influenza viruses (Table 4 and FIG. 16). A smaller panel comprising 18 gene targets (18 central nervous system pathogens) was used to analyze cerebrospinal fluid from individuals with meningitis or encephalitis. Two of, four cases of West Nile virus encephalitis were identified. Fifteen of seventeen cases of enteroviral meningitis were detected representing serotypes CV-B2, CV-B3, CV-B5, E-6, E-11, E-13, E-18, and E-30.

Our results indicate that mass tag PCR is a useful method for molecular characterization of microflora. Sensitivity is similar to real time PCR assays but with the advantage of allowing simultaneous screening for several candidate pathogens. Potential applications include differential diagnosis of infectious diseases, blood product surveillance, forensic microbiology, and biodefense.

FIG. 16 shows the sensitivity of 21-plex mass tag PCR. Dilutions of cloned gene target standards (10 000, 1 000, 500, 100 molecules/assay) diluted in human placenta DNA were analyzed by mass tag PCR. Each reaction mix contained 2× Multiplex PCR Master Mix (Qiagen), the indicated standard and 42 primers at 1×nM concentration labeled with different mass tags. Background in reactions without standard (no template control, 12.5 ng human DNA) was subtracted and the sum of Integrated Ion Current for both tags was plotted.

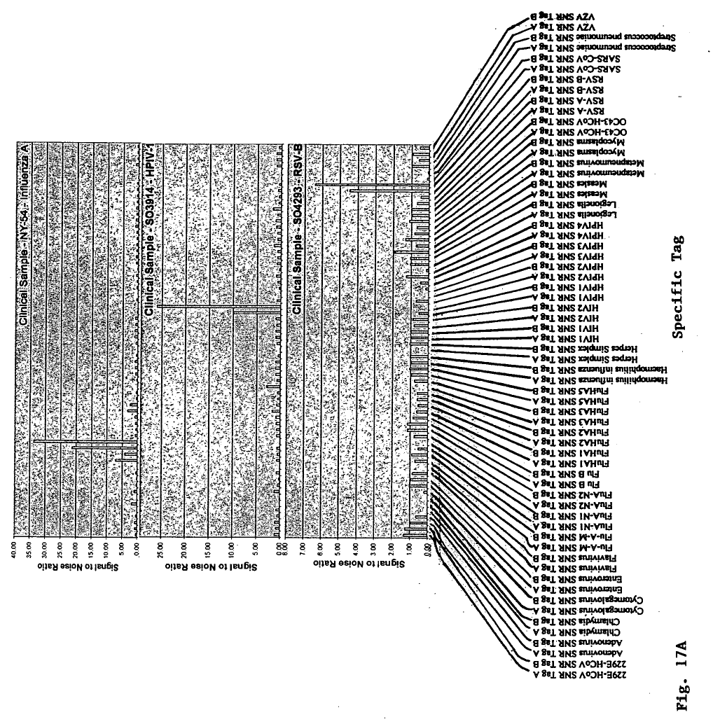

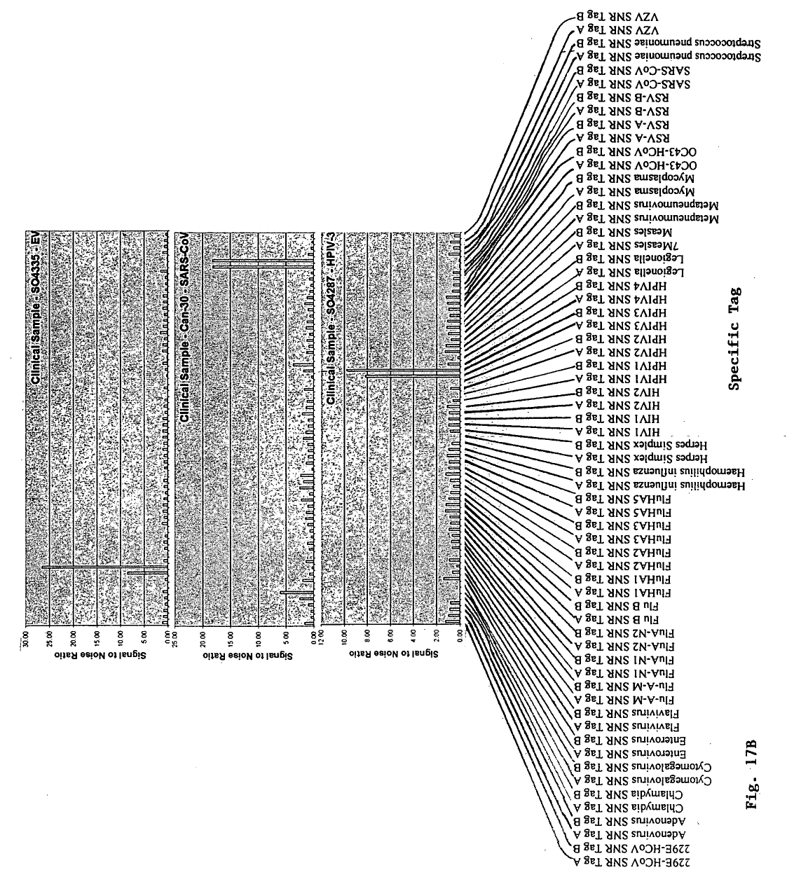

FIG. 17 shows analysis of clinical specimens. (A) Respiratory infection; (B) Encephalitis. RNA from clinical specimens was extracted by standard procedures and reverse transcribed into cDNA (Superscript RT system, Invitrogen, Carlsbad, Calif.; 20 ul volume). Five microliter of reaction was then subjected to mass tag PCR. (A) Detection of Influenza A (H1N1), RSV-B, SARS-CoV, HPIV-3, HPIV-4, and ENTERO using a 31-plex assay including 64 primers targeting Influenza A virus (FLUAV) matrix gene, and for typing H1, H2, H3, H5, N1, and N2 sequence, as well as influenza B virus (FLUBV), respiratory syncytial virus (RSV) groups A and B, human coronaviruses 229E, OC43, and SARS(HCoV-229E, -OC43, and -SARS), human parainfluenza virus (HPIV) types 1, 2, 3, and 4 (groups A and B combined), metapneumovirus, enteroviruses (EV, targeting all serogroups), adenoviruses (HAdV, targeting all serogroups), Mycoplasma pneumoniae, Chlamydia pneumoniae, Legionalla pneumophila, Streptococcus pneumoniae, Haemophilus influenzae, Human herpesvirus 1 (HHV-1, Herpes simplex virus), Human herpesvirus 3 (HHV-3; Varicella-zoster virus), Human herpesvirus 5 (HHV-5, Human cytomegalovirus), Human immunodeficiency virus 1 (HIV-1) and Human immunodeficiency virus 1HIV-2. (B) Detection of ENTERO XX, YY, and ZZ using an 18-plex assay including 36 primers targeting FLUAV matrix gene, H1, H2, H3, H5, N1, and N2 sequence, FLUBV, HCoV 229E, OC43, and SARS, EV, HAdV, HHV-1, -3, and -5, HIV-1, and -2, measles virus (MEV), West Nile virus (WNV), St. Louis virus (SLEV), S. pneumoniae, H. influenzae, and Neisseria meningitides.

TABLE 4
Sensitivity of 22-plex mass tag PCR. Numbers in cells indicate target copy threshold.

| Influenza | Influenza | Influenza | Influenza | Influenza | Influenza | Influenza | Influenza | | | |
|---|---|---|---|---|---|---|---|---|---|---|
| A | A | A | A | A | A | A | B | RSV | RSV | Metapneumo |
| Matrix | N1 | N2 | HA1 | HA2 | HA3 | HA5 | HA | Group A | group B | virus |
| 100 | 100 | 100 | 100 | 100 | 100 | 100 | 500 | 100 | 100 | 100 |
| CoV- | CoV- | CoV- | | | | | | | Enterovirus | Adenovirus |
| SARS | OC43 | 229E | HPIV-1 | HPIV-2 | HPIV-3 | C. pneumoniae | M. pneumoniae | L. pneumophila | (genus) | (genus) |
| 100 | 100 | 100 | 100 | 100 | 100 | 100 | 100 | 100 | 5 000 | 5 000 |

### Example 4

Multiplex PCR

Conventional multiplex PCR assays are established, however, none allow sensitive detection of more than 10 genetic targets. The most sensitive of these assays, real time PCR, is limited to four fluorescent reporter dyes. Gel based systems are cumbersome and limited to visual distinction of products that differ by 20 bp; multiplexing is restricted to the number of products that can be distinguished at 20 bp intervals within the range of 100 to 250 bp (amplification efficiency decreases with larger products); nesting or Southern hybridization is required for high sensitivity. A 9-plex assay has been achieved using hybridization capture enzyme assay.

Disclosed here are panels of nucleic acid sequences to be used in assays for the detection of infectious agents. The sequences include primers for polymerase chain reaction, enzyme sites for initiating isothermal amplification, hybridization selection of nucleic acid targets, as well as templates to serve as controls for validation of these assays. This example focuses on the use of these panels for multiplex mass tag PCR applications. Nucleic acid databases were queried to identify regions of sequence conservation within viral and bacterial taxa wherein primers could be designed that met the following critera: (i) the presence of motifs required to create specific or low degeneracy PCR primers that targeted all members of a microbial group (or subgroup); (ii) Tm of 59-61 C; (iii) GC content of 48-60%; (iv) length of 18-24 bp; (v) no more than three consecutive identical bases; (vi) 3 or more G and/or C residues in the 5′-hexamer; (vii) less than 3 G and/or C residues in the 3′-pentamer; (vii) no propensity for secondary structure (stem-loop) formation; (viii) no inter-primer complementarity that could predispose to primer-dimer formation; (ix) amplification of an 80-250 bp region with no or little secondary structure at 59-61 C. Primers meeting these criteria were then evaluated empirically for equal performance in context of the respective multiplex panel. In the event that no ideal primer candidates could be identified, primers that did not meet one or more of these criteria were synthesized and evaluated for appropriate performance. Those that yielded 80-250 bp amplification products, had Tm of 59-61 C, and showed no primer-dimer artifacts were selected for inclusion into panels.

As a proof-of-principle we designed a panel of primers for detection of 31 target sequences of respiratory pathogens (25-plex respiratory panel) and demonstrated successful detection of all potential targets in a 25-plex PCR reaction. Detection of amplification products was achieved through use of the MASSCODE® technology. Individual primers were conjugated with a unique masscode tag through a photocleavable linkage. Photocleavage of the masscode tag from the purified PCR product and mass spectrometric analysis identifies the amplified target through the two molecular weights assigned to the forward and reverse primer. Primer panels focus on groups of infectious pathogens that are related to differential diagnosis of respiratory disease, encephalitis, or hemorrhagic fevers; screening of blood products; biodefense; food safety; environmental contamination; or forensics.

### Example 5

Background and Significance

The advent of SARS in 2003 poignantly demonstrated the urgency of establishing rapid, sensitive, specific, inexpensive tools for differential laboratory diagnosis of infectious diseases. Through unprecedented global collaborative efforts, the causative agent was rapidly implicated and characterized, facilitating development of serologic and molecular assays for infection, and containment of the outbreak. Nonetheless, as the northern hemisphere entered the winter season of 2004, the diagnosis of SARS still rests on clinical and epidemiological as well as laboratory criteria. The WHO SARS International Reference and Verification Laboratory Network met on Oct. 22, 2003 to review the status of laboratory diagnostics in acute severe pulmonary disease. Quality assurance testing indicated that false positive SARS CoV PCR results were infrequent in network labs. However, participants registered concern that current assays did not allow simultaneous detection of a wide range of pathogens that could aggravate disease or themselves result in clinical presentations similar to SARS.

Methods for cloning nucleic acids of microbial pathogens directly from clinical specimens offer new opportunities to investigate microbial associations in diseases. The power of these methods is not only sensitivity and speed but also the potential to succeed where methods for pathogen identification through serology or cultivation may fail due to absence of specific reagents or fastidious requirements for agent replication.

Various methods are employed or proposed for cultivation-independent characterization of infectious agents. These can be broadly segregated into methods based on direct analysis of microbial nucleic acid sequences, direct analysis of microbial protein sequences, immunological systems for microbe detection, and host response profiling. Any comprehensive armamentarium should include most, if not all, of these tools. Nonetheless, classical methods for microbiology remain important. Indeed, the critical breakthrough during the SARS outbreak was the cultivation of the agent in tissue culture.

Real-time PCR methods have significantly changed diagnostic molecular microbiology by providing rapid, sensitive, specific tools for detecting and quantitating genetic targets. Because closed systems are employed, real-time PCR is less likely than nested PCR to be confounded by assay contamination due to inadvertent aerosol introduction of amplicon/positive control/cDNA templates that can accumulate in diagnostic laboratories. The specificity of real time PCR is both, a strength and a limitation. Although the potential for false positive signal is low so is the utility of the method for screening to detect related but not identical genetic targets. Specificity in real-time PCR is provided by two primers (each approximately 20 matching nucleotides (nt) in length) combined with a specific reporter probe of about 27 nt. The constraints of achieving hybridization at all three sites may confound detection of diverse, rapidly evolving microbial genomes such as those of single-stranded RNA viruses. These constraints can be compensated in part by increasing numbers of primer sets accommodating various templates. However, because real-time PCR relies on fluorescent reporter dyes, the capacity for multiplexing is limited to the number of emission peaks that can be unequivocally separated. At present up to four dyes can be identified simultaneously. Although the repertoire may increase, it will unlikely to change dramatically.

To address the need for enhanced multiplex capacity in diagnostic molecular microbiology we have established a PCR platform based on mass tag reporters that are easily distinguished in MS as discrete signal peaks. Major advantages of the PCR/MS system include: (1) hybridization to only two sites is required (forward and reverse primer binding sites) vs real time PCR where an intermediate third oligonucleotide is used (probe binding site); this enhances flexibility in primer design; (2) tried and proven consensus PCR primers can be adapted to PCR/MS; this reduces the time and resources that must be invested to create new reagents and assay controls; (3) the current repertoire of 60 tags allows highly multiplexed assays; additional tags can be easily synthesized to allow further complexity; and (4) sensitivity of real time PCR is maintained. A limitation of PCR/MS is that it is unlikely to provide more than a semi-quantitative index of microbe burden. Thus, we view PCR/MS as a tool with which to rapidly screen clinical materials for the presence of candidate pathogens. Thereafter, targeted secondary tests, including real time PCR, should be used (to quantitate microbe burden and pursue epidemiologic studies.

Selection of APCI LCMS Platform

Mass spectrometry is a rapid, sensitive method for detection of small molecules. With the development of Ionization techniques such as matrix assisted laser desorption ionization (MALDI) and electrospray ionization (ESI), MS has become a indispensable tool in many areas of biomedical research. Although these ionization methods are suitable for the analysis of bioorganic molecules, such as peptides and proteins, improvements in both detection and sample preparation will be required before mass spectrometry can be used to directly detect long DNA fragments. A major confound in exploiting MS for genetic investigation has been that long DNA molecules are fragmented during the analytic process. The mass tag approach we have developed overcomes this limitation by detecting small stable mass tags that serve as signatures for specific DNA sequences rather than the DNA sequences themselves.

Ionization and detection of the photocleaved mass tags have been extensively characterized using atmospheric pressure chemical ionization (APCI) as the ionization source while using a single quadrupole mass spectrometer as the detector (Jingyue et al., Kim et al. 2003; Kokoris et al. 2000). Because buffer and inorganic salts impact ionization efficiency, performance in ESI was determined to be critically dependent upon sample preparation conditions. In MALDI, matrix must be added prior to sample introduction into the mass spectrometer, which is a time consuming step that requires costly sample spotting instrumentation. Similarly, speed is often limited by the need to search for an ideal irradiation spot to obtain interpretable mass spectra. In contrast, APCI is much more tolerant of residual inorganic salts (than ESI) and does not require mixing with matrix to prepare crystals on a target plate. Thus, mass tag solutions can be injected directly into the MS via a Liquid Chromatography (LC) delivery system. Since mass tags ionize well under APCI conditions and have small mass values (less that 800 amu), they are detected with high sensitivity (<5 femtomolar limit of detection) with the APCI-Quadrupole LCMS platform.

Methods for synthesis and APCI-MS analysis of mass tags coupled to DNA fragments are illustrated in FIG. 1 where precursors are (a) acetophenone; (b) 4-fluoroacetophenone; (c) 3-methoxyacetophenone; and (d) 3,4-dimethoxyacetophenone.

Upon nitration and reduction, the photoactive tags are produced and used to code for the identity of different primer pairs. An example for photocleavage and detection of four tags is shown in FIG. 9. APCI mass spectra for four mass tags after from the corresponding primers (mass tag # 1,2-nitrosoacetophenone, m/z 150; mass tag # 2, 4-fluoro-2-nitrosoacetophenone, m/z 168; mass tag # 3, 5-methoxy-2-nitrosoacetophenone, m/z 180; mass tag # 4, 4,5-dimethoxy-2-nitrosoacetopheone, m/z 210). The four mass tag-labeled primers were mixed together and the mixture was irradiated under UV light (λ˜340 nm) for 5 seconds, introduced into an APCI mass spectrometer and analyzed for the four masses to produce the spectrum. The peak with m/z of 150 is mass-tag 1, 168 is mass-tag 2, 180 is mass-tag 3 and 210 is mass-tag 4.

The mechanism for release of these tags from DNA is shown in FIG. 10. Four mass tag-labeled DNA molecules (Bottom) Chemical structures of the corresponding photocleaved mass tags (2-nitrosoacetophenone, 4-fluoro-2-nitrosoacetophenone, 5-methoxy-2-nitrosoacetophenone and 4,5-dimethoxy-2-nitrosoacetophenone) after UV irradiation at 340 nm.

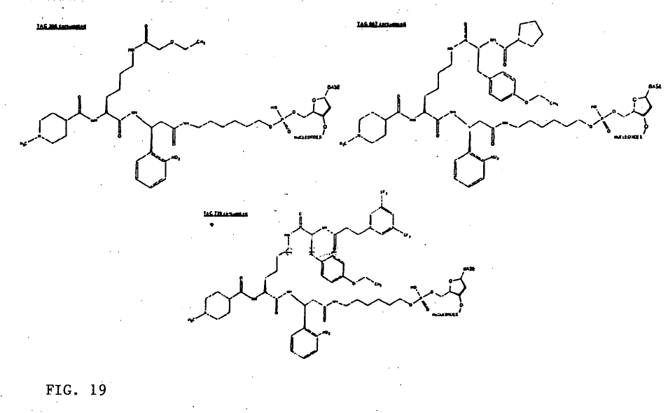

This result indicates that the 4 compounds designed as mass tags are stable and produce discrete high-resolution digital data in an APCI mass spectrometer. In the research plan described below, the unique m/z from each mass tag will translate to the identity of a viral sequence. Qiagen has developed a large library of more than 80 proprietary masscode tags (Kokoris et al. 2000). Examples are shown in FIG. 19.

Establishment of a PCR/MS Assay for Respiratory Pathogens

During the SARS 2003 Beijing outbreak we established a specific and sensitive real time PCR assay for SARS-CoV (Zhai et al, 2004). The assay was extended to allow simultaneous detection of SARS-CoV as well as human coronaviruses OC43 and 229E in light of recent data from China suggesting the potential for coinfection and increased morbidity (FIG. 11). This human coronavirus assay (3 viral genes and 1 housekeeping gene) exhausted the repertoire of fluorescent tags with which to pursue multiplex real time PCR analysis of clinical materials. The importance of extending rapid molecular assays to include other respiratory pathogens is reinforced by the reappearance of SARS in China and reports of a new highly virulent influenza virus strain in Vietnam.

To build a more comprehensive respiratory pathogen surveillance assay we adapted the human coronavirus primers to the PCR/MS platform, and added reagents required to detect other relevant microbes. Influenza A virus was included through a set of established primer sequences obtained through Georg Pauli (Robert Koch Institute, Germany; Schwaiger et al 2000). For the bacterial pathogen M. pneumoniae we also used unmodified primer sequences published for real time PCR (Welti et al 2003) to evaluate their use on the PCR/MS platform. Using a panel of mass tags developed by QIAGEN, pilot experiments were performed, demonstrating the feasibility of detecting several respiratory pathogens in a single multiplexed assay on the PCR/MS platform.

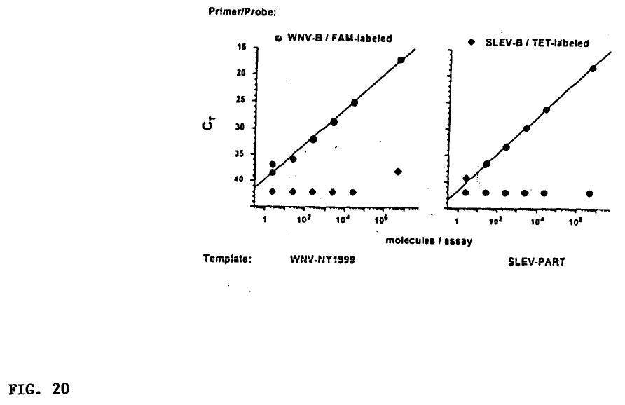

Subsequent to the 1999 West Nile Virus (WNV) outbreak in the U.S. we also built a real time PCR assay for differential diagnosis of flaviviruses WNV and St. Louis encephalitis virus—see FIG. 20. Other validated tools for broad range detection of NIAID priority agents include universal primer stes for detection of Dengue type 1, 2, 3, and 4; various primer sets detecting all members of the bunyamwera and California encephalitis serogroups of the bunyaviruses, see table 13, and not yet validated primer sets for detection of all six Venezuelan equine encephailitis virus serotypoes developed for Molecular Epidemiology, AFEIRA/SDE. Brooks, Tex.

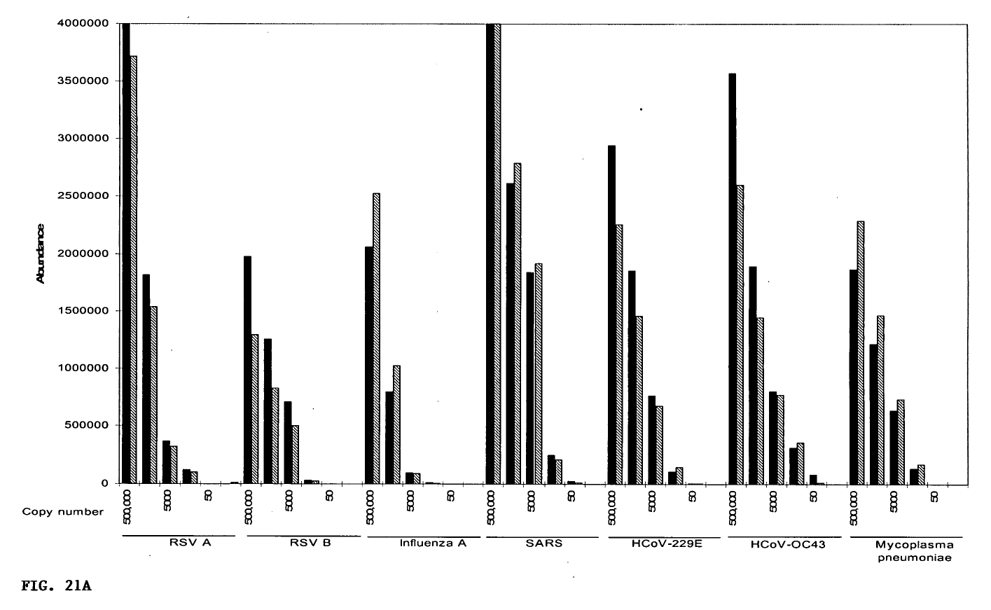

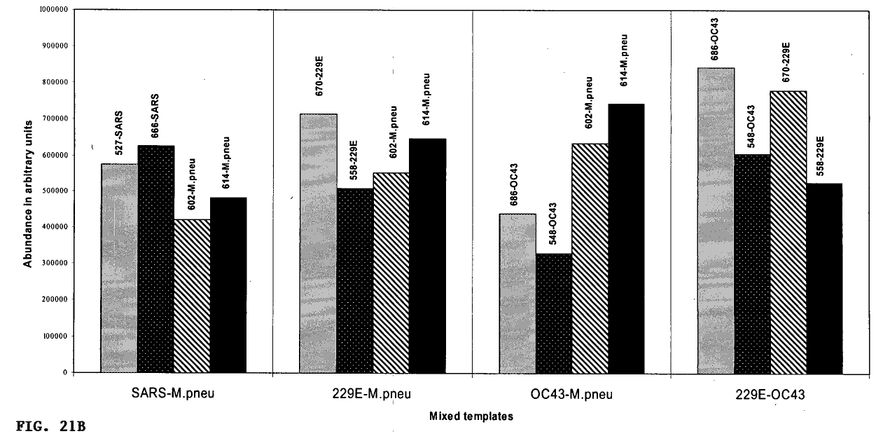

The current Masscode photocleavable mass tag repertoire comprises over 80 tags. FIG. 12 demonstrates the specificity of the mass tag detection approach in an example where 58 different mass tags conjugated to oligonucleotides via a photocleavable linkage were identified after UV cleavage and MS. Each of the 10 primers for the 5-plex assay (SARS-CoV, CoV-229E, CoV-OC43, Influenza A virus, and M. pneumoniae) was conjugated to a different mass tag such that the identity of a given pathogen was encoded by a specific binary signal (e.g. SARS-CoV, forward primer, 527 amu; reverse primer 666 amu; see FIG. 13B). The presence of mass tags did not impair performance of primers in PCR and yielded clear signals for all 5 agents (FIGS. 13A, 13B). No noise was observed using unmodified or mass tag-modified primer sets in a background of 125 ng of normal total human DNA per assay (FIG. 13C). In general, FIG. 13 shows singleplex mass tag PCR for (1) Influenza A virus matrix protein (618 amu fwd-primer, 690 amu rev-primer), human coronaviruses (2) SARS (527/666), (3) 229E (670/558), (4) OC43 (686/548), and the bacterial agent (5) M. pneumoniae (602/614). (6) 100 bp ladder. In subsequent experiments we extended the respiratory pathogen panel to include respiratory syncytial virus groups A and B. Non-optimized pilot studies in this 7-plex system indicated a detection threshold of <500 molecules (FIG. 21). As a test of feasibility for PCR/MS detection of coinfection, mixtures of DNA templates representing two different pathogens were analyzed successful detection of two targets (FIG. 21) confirmed the suitability of this technology for clinical applications where coinfection may be critical to pathogenesis and epidemiology.

Establishment of a Platform for Portable MS

Griffin has developed a portable mass spectrometer that is roughly the size of a tower computer (including vacuum system), weighs less than 50 lbs, and consumes ˜150 W depending on operating conditions. This system has a mass range of 400 Da with unit mass resolution. It has been used to detect part-per-trillion level atmospheric constituents. Included below is a representative spectrum of methyl salicylate collected on a miniature cylindrical ion trap mass analyzer coupled to a corona discharge ionization source (data collected in Prof. R. G. Cooks research laboratory at Purdue University). This data demonstrates the feasibility of using this type of instrumentation to detect the mass tags of interest as well as the specificity of the ionization source. FIG. 14 shows mass spectrum data representative of data collected using a miniature cylindrical ion trap mass analyzer coupled with a corona discharge ionization source. FIG. 15 shows a mass spectrum of perflouro-dimethclcyclohexane collected on a prototype atmospheric sampling glow discharge ionization (ASGDI) source. ASGDI is an external ionization source related to the APCI source proposed here.

Griffin has developed a mass spectrometer for field transportable use. Power consumption, weight, size, and ease of use have been focus design points in the development of this instrument. It has not been designed specifically for interface to an atmospheric pressure ionization (API) source like the one proposed here for pathogen surveillance and discovery. Thus, our focus in this proposal is directed toward the integration of an atmospheric pressure chemical ionization (APCI) source and the required vacuum, engineering, and software considerations associated with this integration.

Experimental Design

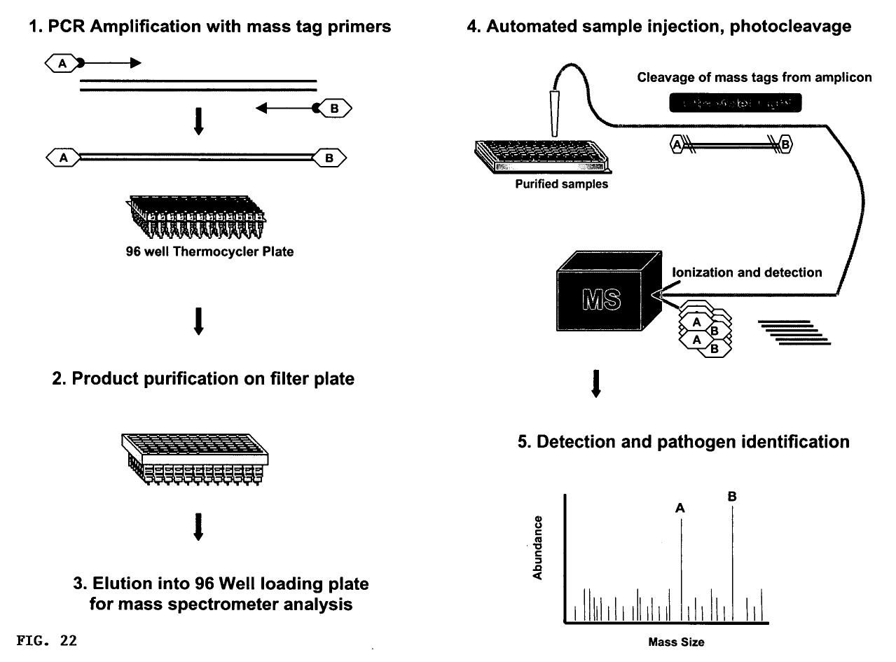

A cartoon of the assay procedure is shown in FIG. 22. Labeled amplification products will be generated during PCR amplification with mass tagged primers. After isolation from non-incorporated primers by binding to silica in Qiagen 96-well or 384-well PCR purification modules, products will be eluted into the injection module of the mass-spectrometer. The products traverse the path of a UV light source prior to entering the nebulizer, releasing photocleavable tags (one each from the forward and reverse primer). Mass tags are then ionized. Analysis of the mass code spectrum defines the pathogen composition of the specimen.

The repertoire of potential pathogens to be targeted during this project is listed in Table 13. Forward and reverse primer pairs for pathogens listed in Table 13 are (reading from top to bottom starting with RSV-A and ending with M. Pneumoniae), SEQ ID NOS:1 and 2, 3 and 4, 9 and 10, 21 and 22, 23 and 24, 26 and 27, and 49 and 50.

TABLE 13

Respiratory Panel Mass-Tag Primers

| | Forward | | Reverse | |
|---|---|---|---|---|
| Pathogen | primer | Sequence | primer | Sequence |
| RSV A | RSA-U1137 | AgATCAACTTCTgTCATCCA | RSV-L1192 | gCACATCATAATTAggAgTATCAAT |
| | | gCAA | | |
| RSV B | RSB-U1248 | AAgATgCAAATCATAAATTC | RSV-1318 | TgATATCCAgCATCTTTAAgTATCT |
| | | ACAggA | | TTATAgTg |
| Influenza A | | | | |
| (N1) | | | | |
| Influenza A | | | | |
| (N2) | | | | |
| Influenza A | AM-U151 | CATggAATggCTAAAgACAAg | AM-L397 | AAgTgCACCAgCAgAATAACTgAg |
| (M) | | ACC | | |
| Influenza B | | | | |
| SARS-CoV | CIID-28891F | AAg CCT CgC CAA AAA CgT | CIID-29100R | AAg TCA gCC ATg TTC CCg AA |
| | | AC | | |
| 229E-CoV | Taq-Co22- | ggC gCA AgA ATT CAg AAC | Taq-Co22- | TAA gAg CCg CAg CAA CTg C |
| | 418F | CA | 636R | |
| OC43-CoV | Taq-Co43- | TgT gCC TAT TgC ACC Agg | Taq-Co43- | CCC gAT CgA CAA TgT CAg C |
| | 270F | AgT | 508R | |
| Metapneumovirus | | | | |
| Parainfluenza | | | | |
| 1 | | | | |
| Parainfluenza | | | | |
| 2 | | | | |
| Parainfluenza | | | | |
| 3 | | | | |
| Parainfluenza | | | | |
| 4 | | | | |
| M. | MTPM1 | CCAACCAAACAACAACgTTC | MTPM2 | ACCTTgACTggAggCCgTTA |
| pneumoniae | | A | | |
| L. | | | | |
| pneumophilae | | | | |
| C. | | | | |
| pneumoniae | | | | |

Design and Synthesize Primers

Missing primers will be designed using the same approach as employed for the 7-plex assay. Available sequences will be extracted from GenBank. Conserved regions suitable for primer design will be identified using standard software programs as well as custom software (patent application XYZ). Primer properties will be assessed by commercial primer selection software including OLIGO (Molecular Biology Insights), Primer Express (PE Applied Biosystems), and Primer Premiere (Premiere Biosoft International). Non-tagged primers will be synthesized, and performance assessed using cloned target sequences as described in preliminary data. Primers will be evaluated for signal strength and specificity against a background of total human DNA. Currently, 80% of primers perform as predicted by our algorithms. Thus, to minimize delay we typically synthesize multiple primer sets for similar genetic targets and evaluate their performance in parallel.

Inherent in the exquisite sensitivity of PCR is the risk of false positive results due to inadvertent introduction of synthetic templates such as those comprising positive control and calibration reagents. Calibration reagents will be components of kits distributed to network laboratories and customers. Thus, to allow recognition of control vs authentic, natural amplification products, we will modify calibration reagents by introducing a restriction enzyme cleavage site in between the primer binding sites through site directed mutagenesis. We have used this approach in projects concerned with epidemiology of viral infection in various chronic diseases including Bornaviruses in neuropsychiatric disease (NIH/MH57467), measles virus in autism (CDC/American Academy of Pediatrics), and enteroviruses in type I diabetes mellitus (NIH/AI55466).

Establish Multiplex Assay Using Cloned Template Standards

Before committing resources to generating mass tagged primers we will test the performance of individual primer sets with unmodified primers. Amplification products in these single assays will be detected by gel electrophoresis. This strategy will not serve for multiplex assays because products of individual primer sets will be similar in size i.e., all will be <300 bp. Although individual products in multiplex assays could be resolved by sequence analysis our experience suggests it will be more cost effective to proceed directly to PCR/MS analysis. Thus, after-performance is confirmed in single assays we will generate mass tagged primers for multiplex analyses. All assays will be optimized first for PCR using serial dilutions of plasmid DNA, and then for RT-PCR using serial dilutions of synthetic transcripts. A multiplex assay will be considered successful if it detects all target sequences at a sensitivity of 50 copies plasmid DNA per assay and 100 copies RNA per assay. Successful multiplex assay performance will also include detection of all permutative combinations of two agents to ensure the feasibility of diagnosing simultaneous infection.

Optimize Multiplex Assay Using Cell Culture Extracts

After establishing performance parameters with calibrated synthetic reagents, cell culture extracts of authentic pathogens will be used. We will recommend specific kits for nucleic acid extraction and RT-PCR. Nonetheless, we recognize that some investigators may choose to use other reagents. Thus, we will assess performance of assays with RNA extracted using readily available commercial systems that do or do not include organic solvents (e.g, Tri-Reagent vs RNeasy). Our current protocol employs Tri-Reagent. Similarly, although we use Superscript reverse transcriptase (Invitrogen) and HotStart polymerase (QIAGEN), we will also assess the performance of ThermoScript RT (Invitrogen) at elevated temperature, and of single-step RT-PCR systems like the Access Kit (Promega). To optimize efficiency where clinical material mass is limited and to reduce the complexity of sample preparation, both viral and bacterial agents will be identified using RT-PCR. In the event network collaborators agree an agent is characterized by substantive phylogenetic diversity, cell culture systems will include at least three divergent isolates of each pathogen. Nasal swabs, sputum and lavage specimens will be spiked with culture material to optimize recovery methods for viral as well as bacterial agents. Assays are validated using banked specimens from naturally infected humans, and naturally infected animals.

### REFERENCES FOR EXAMPLE 5

Briese, T., Jia, X. Y., Huang, C., Grady, L. J., and Lipkin, W. I. (1999). Identification of a Kunjin/West Nile-like flavivirus in brains of patients with New York encephalitis. Lancet 354, 1261-1262.
Briese, T., Rambaut, A., Pathmajeyan, M., Bishara, J., Weinberger, M., Pitlik, S., and Lipkin, W. I. (2002). Phylogenetic analysis of a human isolate from the 2000 Israel West Nile virus epidemic. Emerg Infect Dis 8(5), 528-31.
Briese, T., Schneemann, A., Lewis, A. J., Park, Y. S., Kim, S., Ludwig, H., and Lipkin, W. I. (1994). Genomic organization of Borna disease virus. Proc Natl Acad Sci USA 91(10), 4362-6.
Ju, J., Li, Z., and Itagaki, Y. (2003). Massive parallel method for decoding DNA and RNA. U.S. Pat. No. 6,664,079.
Kim, S., Edwards, J. R., Deng, L., Chung, W., and Ju, J. (2002). Solid phase capturable dideoxynucleotides for multiplex genotyping using mass spectrometry. Nucleic Acids Res 30(16), e85.
Kim, S., Ruparel, H. T., Gilliam, T. C., and Ju, J. (2003). Digital genotyping using molecular affinity and mass spectrometry. Nat Rev Genet 4, 1001-1008.
Kokoris, M., Dix, K., Moynihan, K., Mathis, J., Erwin, B., Grass, P., Hines, B., and Duesterhoeft, A. (2000). High-throughput SNP genotyping with the Masscode system. Mol. Diagn. 5, 329-340.
Li, Z., Bai, X., Ruparel, H., Kim, S., Turro, N. J., and Ju, J. (2003). A photocleavable fluorescent nucleotide for DNA sequencing and analysis. Proc Natl Acad Sci USA 100(2), 414-9.
Lipkin, W. I., Travis, G. H., Carbone, K. M., and Wilson, M. C. (1990). Isolation and characterization of Borna disease agent cDNA clones. Proc Natl Acad Sci USA 87(11), 4184-8.
Schweiger, B., Zadow, I., Heckler, R., Timm, H., and Pauli, G. (2000). Application of a fluorogenic PCR assay for typing and subtyping of influenza viruses in respiratory samples. J Clin Microbiol 38(4), 1552-8.
Walker, M. P., Schlaberg, R., Hays, A. P., Bowser, R., and Lipkin, W. I. (2001). Absence of echovirus sequences in brain and spinal cord of amyotrophic lateral sclerosis patients. Ann Neurol 49(2), 249-53.
Welti, M., Jaton, K., Altwegg, M., Sahli, R., Wenger, A., and Bille, J. (2003). Development of a multiplex real-time quantitative PCR assay to detect Chlamydia pneumoniae, Legionella pneumophila and Mycoplasma pneumoniae in respiratory tract secretions. Diagn Microbiol Infect Dis 45(2), 85-95.
Zhai, J., Briese, T., Dai, E., Wang, X., Pang, X., Du, Z., Liu, H., Wang, J., Wang, H., Guo, Z., Chen, Z., Jiang, L., Zhou, D., Han, Y., Jabado, O., Palacios, G., Lipkin, W. I., and Yang, R. (2004). Real-time polymerase chain reaction for detecting SARS coronavirus, Beijing 2003. Emerg Infect Dis 10, 300-303.

### Example 6

Primer Design and Synthesis, Template Design and Synthesis

Respiratory Panel includes 27 gene targets with validated primer sets as shown below in Table 5. Forward and reverse primer pairs (SEQ ID NOs:1-54) are given for each pathogen (reading from top to bottom starting with RSV-A and ending with C. Pneumoniae). For example, forward primer for RSV-A is SEQ ID NO:1, reverse primer for RSV-A is SEQ ID NO:2. Forward primer for RSV-B is SEQ ID NO:3, reverse primer for RSV-B is SEQ ID NO:4, etcetera.

TABLE 5

Respiratory Panel Mass-Tag Primers

| | Forward | | Reverse | |
|---|---|---|---|---|
| Pathogen | primer | Sequence | primer | Sequence |
| RSV A | RSA-U1137 | AgATCAACTTCTgTCATCCAgC | RSV-L1192 | gCACATCATAATTAggAgTATCAAT |
| | | AA | | |
| RSV B | RSB-U1248 | AAgATgCAAATCATAAATTCAC | RSV-1318 | TgATATCCAgCATCTTTAAgTATCT |
| | | AggA | | TTATAgTg |
| Influenza A | NA1-U1078 | ATggTAATggTgTTTggATAggA | NA1-L1352 | AATgCTgCTCCCACTAgTCCAg |
| (N1) | | Ag | | |
| Influenza A | NA2-U560 | AAgCATggCTgCATgTTTgTg | NA2-L858 | ACCAggATATCgAggATAACAggA |
| (N2) | | | | |
| Influenza A | AM-U151 | CATggAATggCTAAAgACAAgA | AM-L397 | AAgTgCACCAgCAgAATAACTgAg |
| (M) | | CC | | |
| Influenza A | HA1-U583 | ggTgTTCATCACCCgTCTAACA | HA1-L895 | gTgTTTgACACTTCgCgTCACAT |
| (H1) | | T | | |
| Influenza A | H2A208U27 | gCTATgCAAACTAAACggAATY | H2A559L26 | TATTgTTgTACgATCCTTTggCAAC |
| (H2) | | CCTCC | | C |
| Influenza A | HA3-U115 | gCTACTgAgCTggTTCAgAgTT | HA3-L375 | gAAgTCTTCATTgATAAACTCCAg |
| (H3) | | C | | |
| Influenza A | HA5human- | TTACTgTTACACATgCCCAAgA | HA5human- | AggYTTCACTCCATTTAgATCgCA |
| (H5) | u71 | CA | L147 | |
| Influenza B | BHA-U188 | AgACCAgAgggAAACTATgCCC | BHA-L347 | CTgTCgTgCATTATAggAAAgCAC |
| SARS-CoV | CIID-28891F | AAgCCTCgCCAAAAACgTAC | CIID- | AAgTCAgCCATgTTCCCgAA |
| | | | 29100R | |
| 229E-CoV | Taq-Co22- | ggCgCAAgAATTCAgAACCA | Taq-Co22- | TAAgAgCCgCAgCAACTgC |
| | 418F | | 636R | |
| OC43-CoV | Taq-Co43- | TgTgCCTATTgCACCAggAgT | Taq-Co43- | CCCgATCgACAATgTCAgC |
| | 270F | | 508R | |
| Metapneumovirus | MPV01.2 | AACCgTgTACTAAgTgATgCAC | MPV02.2 | CATTgTTTgACCggCCCCATAA |
| European | | TC | | |
| Metapneumovirus | MV-Can-U918 | AAgTCCAAAggCAggRCTgTTA | MV-Can- | CCTgAAgCATTRCCAAgAACAACA |
| Canadian | | TC | L992 | C |
| Parainfluenza | HPIV1-U82 | TACTTTTgACACATTTAgTTCC | HPIV1-L167 | CggTACTTCTTTgACCAggTATAAT |
| 1 | | AggAg | | Tg |
| Parainfluenza | HPIV2-U908 | ggACTTggAACAAgATggCCT | HPIV2-L984 | AgCATgAgAgCYTTTAATTTCTggA |
| 2 | | | | |
| Parainfluenza | HPIV3-U590 | gCTTTCAgACAAgATggAACAg | HPIV3-L668 | gCATKATTgACCCAATCTgATCC |
| 3 | | Tg | | |
| Parainfluenza | HPIV4A-U191 | AACAgAAggAAATgATggTggAA | HPIV4A- | TgCTgTggATgTATgggCAg |
| 4A | | C | L269 | |
| Parainfluenza | HPIV4B-U194 | AgAAgAAAACAACgATgAgACA | HPIV4B- | gTTTCCCTggTTCACTCTCTTCA |
| 4B | | Agg | L306 | |
| Cytomegalovirus | CMV-U421 | TACAgCACgCTCAACACCAAC | CMV-L501 | CCCggCCTTCACCACCAACCgAAA |
| | | gCCT | | A |
| Measles virus | MEA-U1103 | CAAgCATCATgATYgCCATTC | MEA-L1183 | CCTgAATCYCTgCCTATgATgggTT |
| | | CTgg | | T |
| Adenovirus | ADV2F-A | CCCMTTYAACCACCACCg | ADV1R-A | ACATCCTTBCKgAAgTTCCA |
| Enterovirus | 5UTR-U447 | TCCTCCggCCCCTgAATgCggC | 5UTR-L541 | gAAACACggWCACCCAAAgTASTC |
| | | TAATCC | | g |
| M. | MTPM1 | CCAACCAAACAACAACgTTCA | MTPM2 | ACCTTgACTggAggCCgTTA |
| pneumoniae | | | | |
| L. | Legpneu- | gCATWgATgTTARTCCggAAgC | LegPneu- | CggTTAAAgCCAATTgAgCg |
| pneumophilae | U149 | A | L223 | |
| C. | CLPM1 | CATggTgTCATTCgCCAAgT | CLPM2 | CgTgTCgTCCAgCCATTTTA |
| pneumoniae | | | | |

Table 6, NIAID Priority Agent Panel.

Assays have been designed using 4 primer sets and their cognate synthetic Rift Valley Fever, Crimean Congo Hemorrhagic Fever, Ebola Zaire and Marburg virus templates created via PCR using overlapping polynucleotides, as shown in Table 6. Forward and reverse primer pairs (SEQ ID NOs:55-62) are given for four of the listed pathogens (reading from top to bottom starting with Rift Valley Fever virus and ending with Marburg virus). For example, forward primer for Rift Valley Fever virus is SEQ ID NO:55, reverse primer for Rift Valley Fever virus is SEQ ID NO:56. Forward primer for CCHF virus is SEQ ID NO:57, reverse primer for CCHF virus is SEQ ID NO:58, etcetera.

TABLE 6

NIAID Priority Agents Panel Mass-Tag Primers

| | Forward | | Reverse | |
|---|---|---|---|---|
| Pathogen | primer | Sequence | primer | Sequence |
| B. anthracis | | | | |
| Dengue viruses | | | | |
| West Nile virus | | | | |
| Japanese enc. | | | | |
| virus | | | | |
| St. Louis enc. | | | | |
| virus | | | | |
| Yellow Fever | | | | |
| virus | | | | |
| La Crosse virus | | | | |
| California enc. | | | | |
| virus | | | | |
| Rift Valley | RVF-L660 | ggATTgACCTgTgCCTgTTg | RVF-L660 | gCATTAgAAATgTCCTCTTT |
| Fever virus | | C | | TgCTgC |
| CCHF virus | CCHV- | AgAACACgTgCCgCTTACg | CCHV- | CCATTCYTTYTTRAACTCYT |
| | L120 | CCCA | L120 | CAAACCA |
| VEE virus | | | | |
| EEE virus | | | | |
| WEE virus | | | | |
| Ebola virus | EboZA- | AACACCgggTCTTAATTCT | EboZA- | ggTggTAAAATTCCCATAgT |
| | L319 | TATATCAA | L319 | AgTTCTTT |
| Marburg virus | Mar-L372 | TTCCgTCACAAgCCgAAAT | Mar-L372 | TTATTTTAgTTgAgAAAAgAg |
| | | T | | gTTCATgC |
| LCMV | | | | |
| Junin virus | | | | |
| Machupo virus | | | | |
| Variola virus | | | | |

Encephalitis Agent Panel

Table 7 shows primer sets for encephalitis-inducing agents. Forward and reverse primer pairs (SEQ ID NOs:63-96) are given for each pathogen (reading from top to bottom starting with West Nile virus and ending with Enterovirus). For example, forward primer for West Nile virus is SEQ ID NO:63, reverse primer for West Nile virus is SEQ ID NO:64. Forward primer for St. Louis Encephalitis virus is SEQ ID NO:65, reverse primer for St. Louis Encephalitis virus is SEQ ID NO:66, etcetera.

TABLE 7

Encephalitis Agent Panel Mass-Tag Primers

| | Forward | | Reverse | |
|---|---|---|---|---|
| Pathogen | primer | Sequence | primer | Sequence |
| West Nile | DF3-87F | gCTCCgCTgTCCCTgTgA | DF3-156R | CACTCTCCTCCTgCATggATg |
| virus | | | | |
| St. Louis | SLE-D- | CATTTgTTCAgCTgTCCCAgTC | SLE-D- | CTCACCCTTCCCATgAATTg |
| enc. virus | 73F | | 145R | AC |
| Herpes | HSV-U27 | CCCggATgCggTCCAgACgATT | HSV-L121 | CCCgCggAggTTgTACAAAAAA |
| Simplex | | AT | | gCT |
| virus | | | | |
| HIV 1 | SK68i | TTCTTIggAgCAgCIggAAgCACI | SK69i | TTMATgCCCCAgACIgTIAgTT |
| | | ATgg | | ICAACA |
| HIV 2 | HIV2TMF | ggCTgCACgCCCTATgATA | HIV2TMR | TCTgCATggCTgCTTgATg |
| | PR2 | | PR2 | |
| N. | Nmen- | TCTgAAgCCATTggCCgT | Nmen- | CCAAACACACCACgCgCAT |
| meningitidis | U829 | | L892 | |
| S. | SPPLY- | AgCgATAgCTTTCTCCAAgTgg | SPPLY- | CTTAgCCAACAAATCgTTTA |
| pneumoniae | U532 | | L606 | CCg |
| H. influenzae | HINF-U82 | AAgCTCCTTgMATTTTTTgTAT | Hinf-L158 | gCTgAATTggCTTRgATACCg |
| | | TAgAA | | Ag |
| Influenza B | BHA-U188 | AgACCAgAgggAAACTATgCCC | BHA-L347 | CTgTCgTgCATTATAggAAAg |
| | | | | CAC |
| SARS-CoV | CIID- | AAgCCTCgCCAAAAACgTAC | CIID- | AAgTCAgCCATgTTCCCgAA |
| | 28891F | | 29100R | |
| 229E-CoV | Taq-Co22- | ggCgCAAgAATTCAgAACCA | Taq-Co22- | TAAgAgCCgCAgCAACTgC |
| | 418F | | 636R | |
| OC43-CoV | Taq-Co43- | TgTgCCTATTgCACCAggAgT | Taq-Co43- | CCCgATCgACAATgTCAgC |
| | 270F | | 508R | |
| Cytomegalovirus | CMV- | TACAgCACgCTCAACACCAAC | CMV-L501 | CCCggCCTTCACCACCAACC |
| | U421 | gCCT | | gAAAA |
| Varicella | VZV-U138 | ACgTggATCgTCggATCAgTTgT | VZV-L196 | TCgCTATgTgCTAAAACACgC |
| Zoster virus | | | | gg |
| Measles | MEA- | CAAgCATCATgATYgCCATTCC | MEA- | CCTgAATCYCTgCCTATgATg |
| virus | U1103 | Tgg | L1183 | ggTTT |
| Adenovirus | ADV2F-A | CCCMTTYAACCACCACCg | ADV1R-A | ACATCCTTBCKgAAgTTCCA |
| Enterovirus | 5UTR- | TCCTCCggCCCCTgAATgCggC | 5UTR- | gAAACACggWCACCCAAAgT |
| | U447 | TAATCC | L541 | ASTCg |

Improvements in Multiplexing

Initially, multiplex detection of 7 respiratory pathogen targets at 500 copy sensitivity: RSV group A, RSV group B, Influenza A, HCoV-SARS, HCoV-229E, HCoV-OC43, and M. pneumoniae was determined. Subsequently, sensitivity was improved. Detection at 100 copy sensitivity has been confirmed for 18 respiratory pathogen targets in a 20-plex assay (Table 8). Two of 20 targets, the influenza A M gene and influenza H1 gene, were detected at 500 copies. This typically corresponds in our laboratory to <0.001 TCID₅₀ per assay, a threshold comparable to many useful microbiological assays.

TABLE 8
Sensitivity of respiratory panel

| | | | Influenza | Influenza A | Influenza A | Influenza | Influenza | Influenza A | Influenza A | Influenza |
|---|---|---|---|---|---|---|---|---|---|---|
| | RSV A | RSV B | A (N1) | (N2) | (matrix) | A (H1) | A (H2) | (H3) | (H5) | B |
| 500 | + | + | + | + | + | + | + | + | + | + |
| copies | | | | | | | | | | |
| 100 | + | + | + | + | − | − | + | + | + | + |
| copies | | | | | | | | | | |
| | HCoV- | HCoV- | HCoV- | Metapneumo- | | | | | | |
| | SARS | 229E | OC43 | virus (Eur.) | HPIV-1 | HPIV-2 | HPIV-3 | M. pneumoniae | C. pneumoniae | L. pneumophilae |
| 500 | + | + | + | + | + | + | + | + | + | + |
| copies | | | | | | | | | | |
| 100 | + | + | + | + | + | + | + | + | + | + |
| copies | | | | | | | | | | |

Clinical Samples

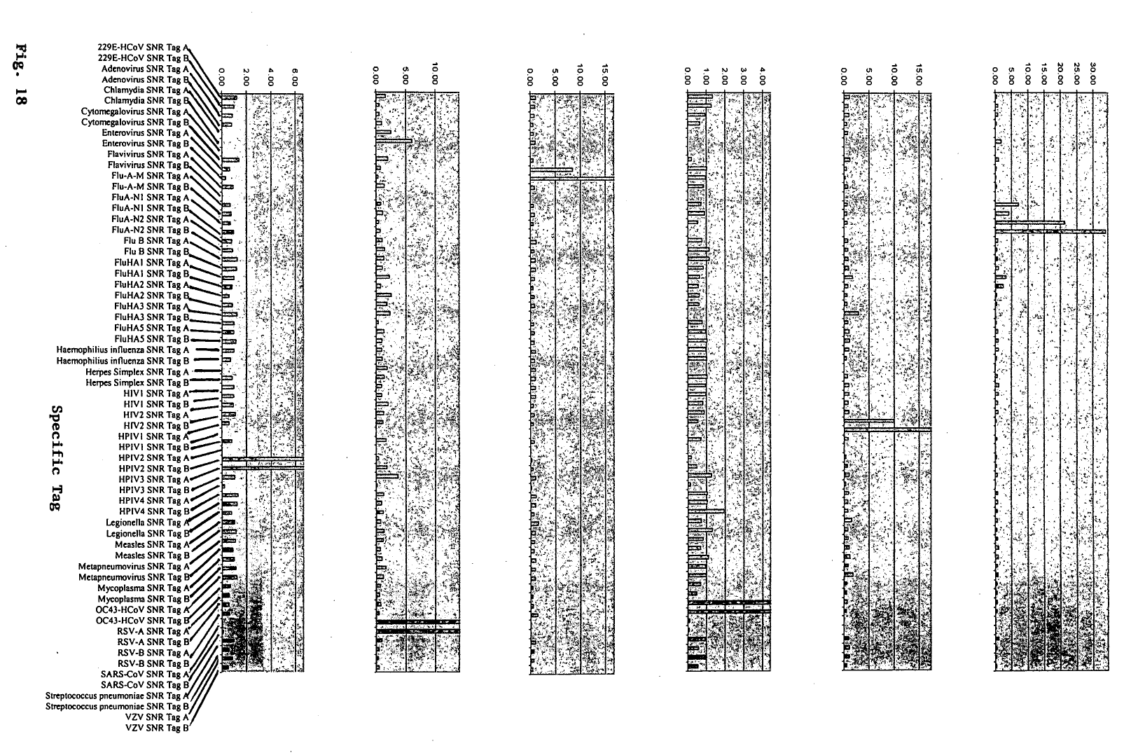

Although assays of synthetic targets were optimized in a complex background of normal tissue nucleic acids, analysis of clinical materials was performed. Banked clinical respiratory specimens were obtained from Cinnia Huang of the Wadsworth Laboratory of the New York State Department of Health and Pilar Perez-Brena of the National Center for Microbiology of Spain. Organisms included: metapneumovirus (n=3), RSV-B (n=3), RSV-A (n=2), adenovirus (n=2), HPIV-1 (n=1), HPIV-3 (n=2), HPIV-4 (n=2), enterovirus (n=2), SARS-CoV (n=4), influenza A (n=2). Six representative results are shown in FIG. 18; Multiplex Mass Tag PCR analysis of six human respiratory specimens. Signal to noise ratio is on the ordinate and primer sets are listed on the abscissa. Mass Tag primer sets employed in a single tube assay are indicated at the bottom of the figure. FIG. 18A—Influenza A (N1, M, H1) H1); 18B—Human Parainfluenza Type 1; 18C—Respiratory Syncytial Group B; 18D—Enterovirus; 18E—SARS CoV; and 18F—Human Parainfluenza Type 3.

Pathogens

Tables 9-12 show a non-comprehenisve list of various target pathogens and corresponding primer sequences. In Table 10, the forward and reverse primer pairs for Cytomegalovirus, SEQ ID NOS: 87 and 88; for HPIV-4A, SEQ ID NOS: 37 and 38; for HPIV-4B, SEQ ID NOS: 39 and 40; for Measles, SEQ ID NOS: 91 and 92; for Varicella Zoster virus, SEQ ID NOS: 89 and 90; for HIV-1, SEQ ID NOS: 69 and 70; for HIV-2, SEQ ID NOS: 71 and 72; for S. Pneumoniae, SEQ ID NOS: 100 and 101; for Haemophilus Influenzae, SEQ ID NOS: 77 and 78; for Herpes Simplex, SEQ ID NOS: 67 and 68; for MV Canadian isolates, SEQ ID NOS: 29 and 30; for Adenovirus 2 A/B 505/630, SEQ ID NOS: 93 and 94; for Enterovirus A/B 702/495, SEQ ID NOS: 95 and 96; and forward primers for Enterovirus A/B 702/495, SEQ ID NOS: 98 and 99.

TABLE 9

| Primer sequence | Name | Target | Previous Masscode | Panel | |
|---|---|---|---|---|---|
| HIV2 | HIV2TMFPR2 | | 586 | Respiratory/Enc | 30 |
| HIV2 | HIV2TMRPR2 | | 570 | Respiratory/Enc | |
| Streptococcus pneumoniae | SPPLY-U532 | Forward A | 714 | Respiratory/Enc | 31 |
| Streptococcus pneumoniae | SPPLY-L606 | Reverse B | 694 | Respiratory/Enc | |
| Haemophilus influenza | HINF-U82 | Forward A | 734 | Respiratory/Enc | 32 |
| Haemophilus influenza | Hinf-L158 | Reverse B | 726 | Respiratory/Enc | |
| Herpes Simplex | HSV-U27 | Forward A | 722 | Respiratory/Enc | 33 |
| Herpes Simplex | HSV-L121 | Reverse B | 706 | Respiratory/Enc | |
| Metaneumovirus Canadian | MV-Can-U918 | Forward A | 718 | Respiratory | 34 |
| Metaneumovirus Canadian | MV-Can-L992 | Reverse B | 654 | Respiratory | |
| Adenovirus | ADV2F-A | Forward A | 503 | Respiratory/Enc | 12 |
| Adenovirus | ADV1R-A | Reverse B | 630 | Respiratory/Enc | |
| Enterovirus | 5UTR-U447 | Forward A | 702 | Respiratory/Enc | 14 |
| Enterovirus | 5UTR-U450 | Forward A | 702 | Respiratory/Enc | |
| Enterovirus | 5UTR-u457 | Forward A | 702 | Respiratory/Enc | 14 |
| Enterovirus | 5UTR-L541 | Reverse B | 495 | Respiratory/Enc | |
| Neisseria meningitidis | Nmen-U829 | Forward A | 730 | Encephalitis/Resp | |
| Neisseria meningitidis | Nmen-L892 | Reverse B | 439 | Encephalitis/Resp | |
| WNV1 | DF3-87F | Forward A | 539 | Encephalitis | |
| WNV1 | DF3-156R | Reverse B | 499 | Encephalitis | |
| WNV2 | WN-Ax-FWD | Forward A | 539 | Encephalitis | |
| WNV2 | WN-Ax-REV | Reverse B | 499 | Encephalitis | |
| SLE | SLE-D-73F | Forward A | 658 | Encephalitis | |
| SLE | SLE-D-145R | Reverse B | 642 | Encephalitis | |
| Cytomegalovirus | CMV-U421 | Forward A | 626 | Respiratory/Enc | 24 |
| Cytomegalovirus | CMV-L501 | Reverse B | 610 | Respiratory/Enc | |
| HPIV4A | HPIV4A-U191 | Forward A | 622 | Respiratory | 25 |
| HPIV4a | HPIV4A-L269 | Reverse B | 606 | Respiratory | |
| HPIV4B | HPIV4B-U194 | Forward A | 622 | Respiratory | 26 |
| HPIV4b | HPIV4B-L306 | Reverse B | 606 | Respiratory | |
| Measles | MEA-U1103 | Forward A | 578 | Respiratory/Enc | 27 |
| Measles | MEA-L1183 | Reverse B | 562 | Respiratory/Enc | |
| VZV | VZV-U138 | Forward A | 515 | Respiratory/Enc | 28 |
| VZV | VZV-L196 | Reverse B | 471 | Respiratory/Enc | |
| HIV1 | SK68i | | 574 | Respiratory/Enc | 29 |
| HIV1 | SK69i | | 383 | Respiratory/Enc | |
| RSV A gen N | RSA-U1137 | Forward A | 467 | Respiratory | 1 |
| RSV A gen N | RSV-L1192 | Reverse B | 455 | Respiratory | |
| RSV B gen N | RSB-U1248 | Forward A | 483 | Respiratory | 2 |
| RSV B gen N | RSV-1318 | Reverse B | 479 | Respiratory | |
| Flu A - N1 | NA1-U1078 | Forward A | 499 | Respiratory | 3 |
| Flu A - N1 | NA1-L1352 | Reverse B | 439 | Respiratory | |
| Flu A - N2 | NA2-U560 | Forward A | 658 | Respiratory | 4 |
| Flu A - N2 | NA2-L858 | Reverse B | 730 | Respiratory | |
| Flu A (MATRIX) | AM-U151 | Forward A | 618 | Respiratory/Enc | 5 |
| Flu A (MATRIX) | AM-L397 | Reverse B | 690 | Respiratory/Enc | |
| Flu B | BHA-U188 | Forward A | 698 | Respiratory/Enc | 6 |
| Flu B | BHA-L347 | Reverse B | 598 | Respiratory/Enc | |
| SARS-Coronavirus | CIID-28891F | Forward A | 527 | Respiratory/Enc | 7 |
| SARS-Coronavirus | CIID-29100R | Reverse B | 666 | Respiratory/Enc | |
| 229E-Coronavirus | Taq-Co22-418F | ForwardA | 670 | Respiratory/Enc | 8 |
| 229E-Coronavirus | Taq-Co22-636R | Reverse B | 558 | Respiratory/Enc | |
| OC43-Coronavirus | Taq-Co43-270F | ForwardA | 686 | Respiratory/Enc | 9 |
| OC43-Coronavirus | Taq-Co43-508R | Reverse B | 548 | Respiratory/Enc | |
| Metapneumovirus | MPV01.2 | ForwardA | 718 | Respiratory | 10 |
| Metapneumovirus | MPV02.2 | Reverse B | 654 | Respiratory | |
| Mycoplasma pneumoniae | MTPM1 | Forward A | 602 | Respiratory | 11 |
| Mycoplasma pneumoniae | MTPM2 | Reverse B | 614 | Respiratory | |
| adenovirus | ADV1F-A | Forward A | 503 | Respiratory/Enc | 12 |
| adenovirus | ADV2R-A | Reverse B | 630 | Respiratory/Enc | |
| Chlamydia | CLPM1 | Forward A | 519 | Respiratory | 13 |
| Chlamydia | CLPM2 | Reverse B | 371 | Respiratory | |
| enterovirus | EV1f | Forward A | 702 | Respiratory/Enc | 14 |
| enterovirus | EV1r | Reverse B | 495 | Respiratory/Enc | |
| flavivirus1 | Fla-U9093 | Forward A | 710 | Encephalitis | 15 |
| flavivirus1 | Fla-L9279 | Reverse B | 594 | Encephalitis | |
| flavivirus2 | Fla-U9954 | Forward A | 710 | Encephalitis | 15 |
| flavivirus2 | Fla-L10098 | Reverse B | 594 | Encephalitis | |
| fluHA1 | HA1-U583 | Forward A | 650 | Respiratory | 16 |
| fluHA1 | HA1-L895 | Reverse B | 634 | Respiratory | |
| fluHA2 | H2A208U27 | Forward A | 662 | Respiratory | 17 |
| fluHA2 | H2A559L26 | Reverse B | 638 | Respiratory | |
| fluHA3 | HA3-U115 | Forward A | 375 | Respiratory | 18 |
| fluHA3 | HA3-L380 | Reverse B | 475 | Respiratory | |
| fluHA5 | HA5-u71 | Forward A | 646 | Respiratory | 19 |
| fluHA5 | HA5-L147 | Reverse B | 395 | Respiratory | |
| HPIV1 | HPIV1-U82 | Forward A | 566 | Respiratory | 20 |
| HPIV1 | HPIV1-L167 | Reverse B | 357 | Respiratory | |
| HPIV2 | HPIV2-U908 | Forward A | 483 | Respiratory | 21 |
| HPIV2 | HPIV2-L984 | Reverse B | 590 | Respiratory | |
| HPIV3 | HPIV3-U590 | Forward A | 642 | Respiratory | 22 |
| HPIV3 | HPIV3-L668 | Reverse B | 539 | Respiratory | |
| Legionella1 | Legpneu-U149 | Forward A | 678 | Respiratory | 23 |
| Legionella1 | LegPneu-L223 | Reverse B | 582 | Respiratory | |

TABLE 10

Respiratory Panel
Mass-Tag Primers

| | | | | | | | |
|---|---|---|---|---|---|---|---|
| Tagged | | Stand- | Primer | | | | |
| Pairs | Tier | ards | Name | Start | Length | Tm | Primer forward |

| | | | | | | | |
|---|---|---|---|---|---|---|---|
| CYTO- | 1 | YES | CMV-U421 | 421 | 25 | 64.51 | TACAGCACGCTCAACACCAACGCCT |
| MEGALO- | | | | | | | |
| VIRUS | | | | | | | |
| HPIV-4A | 1 | clon- | HPIV4A-U191 | 191 | 24 | 59 | AACAGAAGGAAATGATGGTGGAAC |
| | | ing | | | | | |
| HPIV-4B | 1 | clon- | HPIV4B-U194 | 194 | 25 | 59 | AGAAGAAAACAACGATGAGACAAGG |
| | | ing | | | | | |
| MEASLES | 1 | syn- | MEA-U1103 | 1103 | 25 | 59.33 | CAAGCATCATGATYGCCATTCCTGG |
| | | thetic | | | | | |
| VARI- | 1 | YES | VZV-U138 | 138 | 23 | 59.84 | ACGTGGATCGTCGGATCAGTTGT |
| CELLA | | | | | | | |
| ZOSTER | | | | | | | |
| VIRUS | | | | | | | |
| HIV1 | 1 | Thomas | SK68i | SK68i | 28 | 70 to 75 | TTC TTI GGA GCA GCI GGA AGC |
| | | | | | | | ACI ATG G |
| HIV2 | 1 | syn- | HIV2TMFPR2 | hiv2tmfpr2 | 18 | | GGCTGCACGCCCTATGATA |
| | | thetic | | | | | |
| STREPTO- | 1 | syn- | SPPLY-U532 | 532 | 22 | 59 | AGCGATAGCTTTCTCCAAGTGG |
| COCCUS | | thetic | | | | | |
| PNEUMON- | | | | | | | |
| IAE | | | | | | | |
| HAEMO- | 1 | syn- | HINF-U82 | 82 | 27 | 59 | AAGCTCCTTGMATTTTTTGTATTAGAA |
| PHILUS | | thetic | | | | | |
| INFLUEN- | | | | | | | |
| ZAE | | | | | | | |
| HERPES | 1 | YES | HSV-U27 | 27 | 24 | 62.09 | CCCGGATGCGGTCCAGACGATTAT |
| SIMPLEX | | | | | | | |
| MV-Cana- | 1 | syn- | MV-Can-U918 | 918 | 24 | 59 | AAGTCCAAAGGCAGGRCTGTTATC |
| dian | | thetic | | | | | |
| isolates | | | | | | | |
| Adeno- | 1 | YES | ADV2F-A | ADV2F-A | | 58 TO 81 | CCCMTTYAACCACCACCG |
| virus2 | | | | | | | |
| A/B 503/ | | | | | | | |
| 630 | | | | | | | |
| Entero- | 1 | YES | 5UTR-U447 | 447 | | 76 | TCCTCCGGCCCCTGAATGCGGCTAATCC |
| virus | | | | | | | |
| A/B 702/ | | | | | | | |
| 495 | | | | | | | |
| Entero- | 1 | YES | 5UTR-U450 | 450 | | 72 | TCCGGCCCCTGAATGCGGCTAATCC |
| virus | | | | | | | |
| A/B 702/ | | | | | | | |
| 495 | | | | | | | |
| Entero- | 1 | YES | 5UTR-457 | 457 | | 83 | CCCCTGAATGCGGCTAATCC |
| virus | | | | | | | |
| A/B 702/ | | | | | | | |
| 495 | | | | | | | |

| | | | | |
|---|---|---|---|---|
| Tagged | | | | |
| Pairs | Start | Length | Tm | Primer reverse |

| | | | | | | | |
|---|---|---|---|---|---|---|---|
| CYTO- | CMV-L501 | 501 | 25 | 65.08 | CCC GGC CTT CAC CAC CAA CCG | | GIDL |
| MEGALO- | | | | | AAA A | | |
| VIRUS | | | | | | | |
| HPIV-4A | HPIV4A-L269 | 269 | 20 | 59 | TGCTGTGGATGTATGGGCAG | GIDL | |
| HPIV-4B | HPIV4B-L306 | 306 | 23 | 58 | GTTTCCCTGGTTCACTCTCTTCA | GIDL | |
| MEASLES | MEA-L1183 | 1183 | 28 | 58.98 | CCT GAA TCY CTG CCT ATG ATG | GIDL | |
| | | | | | GGT TT | | |
| VARI- | VZV-L196 | 196 | 23 | 59.97 | TCG CTA TGT GCT AAA ACA CGC | GIDL | |
| CELLA | | | | | GG | | |
| ZOSTER | | | | | | | |
| VIRUS | | | | | | | |
| HIV1 | SK69i | SK69i | 26 | | TTMATGCCCCAGACIGTIAGTTICAACA | H | Robert Koch |
| | | | | | | Etterbrok | Institute |
| HIV2 | HIV2TMRPR2 | | | | TCTGCATGGCTGCTTGATG | Schulten, | JVM 88 |
| | | | | | | M | (2000) |
| | | | | | | | 81-87 |
| STREPTO- | SPPLY-L606 | 606 | 23 | 59 | CTTAGCCAACAAATCGTTTACCG | GIDL | |
| CCUS | | | | | | | |
| PHEUMON- | | | | | | | |
| IAE | | | | | | | |
| HAEMO- | Hin1-L158 | 158 | 23 | 58 | GCTGAATTGGCTTRGATACCGAG | GIDL | |
| PHILUS | | | | | | | |
| INFLUEN- | | | | | | | |
| ZAE | | | | | | | |
| HERPES | HSV-L121 | 121 | 24 | 61.55 | CCC GCG GAG GTT GTA CAA AAA | GIDL | |
| SIMPLEX | | | | | GCT | | |
| MV- | MV-Can-L992 | 992 | 25 | 60 | CCTGAAGCATTRCCAAGAACAACAC | GIDL | |
| Canadian | | | | | | | |
| isolates | | | | | | | |
| Adeno- | ADV1R-A | ADV1R-A | | 54 TO 58 | ACATCCTTBCKGAAGTTCCA | Ana | VM 92 |
| virus2 | | | | | | Avetton | (2001) 113- |
| A/B 503/ | | | | | | | 120 |
| 630 | | | | | | | |
| Entero- | 5UTR-L541 | 5UTR-L541 | | 67 T0 87 | GAAACACGGWCACCCAAAGTASTCG | | |
| Virus | | | | | | | |
| A/B 702/ | | | | | | | |
| 495 | | | | | | | |
| Entero- | | | | | | | |
| Virus | | | | | | | |
| A/B 702/ | | | | | | | |
| 495 | | | | | | | |
| Entero- | | | | | | | |
| Virus | | | | | | | |
| A/B 702/ | | | | | | | |
| 495 | | | | | | | |

TABLE 11

| | | | | |
|---|---|---|---|---|
| Tagged Pairs | Standards | LIST OF PRIMERS | Name FWD | Forward-A |

| | | | | |
|---|---|---|---|---|
| RSVA-1A/B 467/455 | YES | RSV A gen N | RSA-U1137 | AGATAACTTCTGTCATCCAGCAA |
| | | RSV A gen N | rsh1ce.fa-777F | GGTGCAGGGCAAGTGATGTTA |
| | | RSV A gen P | RSHP1.fa-235F | CAGGGAACAAGCCCAATTATCA |
| RSVB-1A/B 483/479 | YES | RSV B gen N | RSB-U1248 | AAGATGCAAATCATAAATTCACAGGA |
| | YES | RSV B gen N | rshbcnp.fa-775F | ATGGTTCAGGGCAAGTAATGCT |
| | YES | RSV B gen P | RSHPQ.fa-189F | TCTGGCACCAACATCATCAATC |
| FluA-N1 A/B 499/439 | YES | N1 | NA1-U1078 | ATGGTAATGGTGTTTGGATAGGAAG |
| FluA-N2 A/B 658/730 | YES | N2 | NA2-U560 | AAGCATGGCTGCATGTTTGTG |
| FLuA-M A/B 618/690 | YES | A (MATRIX) | AM-U151 | CATGGAATGGCTAAAGACAAGACC |
| FluB A/B 698/598 | YES | B | BHA-U188 | AGACCAGAGGGAAACTATGCCC |
| | YES | B | | |
| SARS A/B 527/666 | YES | SARS-Coronavirus | CIID-28891F | AAg CCT CgC CAA AAA CgT AC |
| 229E A/B 670/558 | YES | 229E-Coronavirus | Taq-Co22-418F | ggC gCA AgA ATT CAg AAC CA |
| OC43 A/B 686/548 | YES | OC43-Coronavirus | Taq-Co43-270F | TgT gCC TAT TgC ACC Agg AgT |
| Melapnuemo A/B 718/654 | YES | Melapneumovirus | MPV01.2 | AACCGTGTACTAAGTGATGCACTC |
| Mycoplasma - 1 A/B 602/614 | YES | Mycoplasma1 | MTPM1 | CCAACCAAACAACAACGTTCA |
| | | Mycoplasma2 | MpnA | CCGCGAAGAGCAATGAAAAACTCC |
| HPIV1 A/B 566/357 | YES | Parainfluenza 1 | HPIV1-U82 | TACTTTTGACACATTTAGTTCCAGGAG |
| HPIV2 A/B 566/357 | YES | Parainfluenza 2 | HPIV2-U908 | GGACTTGGAACAAGATGGCCT |
| HPIV3 A/B 566/357 | YES | Parainfluenza 3 | HPIV3-U590 | GCTTTCAGACAAGATGGAACAGTG |
| Legionella 1 A/B 678/582 | YES | Legionella1 | Legpneu-U149 | GCATWGATGTTARTCCGGAAGCA |
| | YES | Legionella2 | LGPM1 | AAA GGC ATG CAA GAC GCT ATG |
| | | Legionella3 | LgnA | GGCGACTATAGCGATTTGGAA |
| Chlamydia A/B 519/383 | YES | Chlamydia pneumoniae | CLPM1 | CAT GGT GTC ATT CGC CAA GT |
| FluHA1 A/B 650/590 | YES | HA1 | HA1-U583 | GGTGTTCATCACCCGTCTAACAT |
| FluHA2 A/B 662/539 | YES | HA2 | H2A208U27 | GCTATGCAAACTAAACGGAATYCCTCC |
| FluHA3-1 A/B 586/475 | YES | HA3 | HA3-U115 | GCTACTGAGCTGGTTCAGAGTTC |
| FluHA3-2 A/B 586/475 | YES | HA3 | HA3-U115 | GCTACTGAGCTGGTTCAGAGTTC |
| FluHA5 A/B 646/395 | YES | HA5-human | HA5human-u71 | TTACTGTTACACATGCCCAAGACA |

| | | | | | |
|---|---|---|---|---|---|
| | | | | Tm | Product |
| Tagged Pairs | Tm primer | Name REV | Reverse-B | primer | Size |

| | | | | | |
|---|---|---|---|---|---|
| RSVA-1A/B 467/455 | 62 | RSV-L1192 | GCACATCATAATTAGGAGTATCAAT | 56 | 80 |
| | 63 | rsh1ce.la-1013R | GCCAGCAGCATTGCCTAATAC | 62 | 240 |
| | 63 | RSHP1.la-540R | CTCTTAAACCAACCATGGCATCTC | 63 | 320 |
| RSVB-1A/B 483/479 | 62 | RSV-1318 | TGATATCCAGCATCTTTAAGTATCTTTATAGTG | 62 | 105 |
| | 62 | rshbcnp.fa-913R | TCTCCTCCCAACTTCTGTGCA | 63 | 180 |
| | 63 | RSHPQ.fa-295R | GGGGTGAGATCTTCTTTGAAGCT | 62 | 120 |
| FluA-N1 A/B 499/439 | 61 | NA1-L1352 | AATGCTGCTCCCACTAGTCCAG | 63 | 274 |
| FluA-N2 A/B 658/730 | 64 | NA2-L858 | ACCAGGATATCGAGGATAACAGGA | 62 | 298 |
| FLuA-M A/B 618/690 | 63 | AM-L397 | AAGTGCACCAGCAGAATAACTCAG | 62 | 246 |
| FluB A/B 698/598 | 63 | BHA-L347 | CTGTCGTGCATTATAGGAAAGCAC | 62 | 159 |
| SARS A/B 527/666 | 62 | CIID-2910R | AAg TCA gCC ATg TTC CCg AA | 63 | 130 |
| 229E A/B 670/558 | 64 | Taq-Co22.636R | TAA gAg CCg CAg CAA CTg C | 63 | 240 |
| OC43 A/B 686/548 | 63 | Taq-Co43-508R | CCC gAT CgA CAA TgY CAg C | 63 | 260 |
| Melapnuemo A/B 718/654 | 60 | MPV02.2 | CATTGTTTGACCGGCCCCCATAA | 68 | 205 |
| Mycoplasma - 1 A/B 602/614 | 62 | MTPM2 | ACCTTGACTGGAGGCCGTTA | 62 | 76 |
| | 60 | MpnB | TCGAGGCGGATCATTTGGGGAGGT | 63 | 380 |
| HPIV1 A/B 566/357 | 61 | HPIV1-L167 | CGGTACTTCTTTGACCAGGTATAATTG | 62 | 110 |
| HPIV2 A/B 566/357 | 63 | HPIV2-L964 | AGCATGAGAGCYTTTAATTTCTGGA | 63 | 102 |
| HPIV3 A/B 566/357 | 62 | HPIV3-L668 | GCATKATTGACCCAATCTGATCC | 63 | 103 |
| Legionella 1 A/B 678/582 | 66 | LegPneu-L223 | CGGTTAAAGCCAATTGAGCG | 63 | 79 |
| | 63 | LGPM2 | TGT TAA GAA CGT CTT TCA TTT GCT G | 62 | 75 |
| | 56 | LgnB | GCGATGACCTACTTTCGCATGA | 56 | 100 |
| Chlamydia A/B 519/383 | 62 | CLPM2 | CGT GTC GTC CAG CCA TTT TA | 62 | 85 |
| FluHA1 A/B 650/590 | 62 | HA1-L895 | GTGTTGACACTTCGCGTCACAT | 65 | 312 |
| FluHA2 A/B 662/539 | 67 | H2A559L26 | TATTGTTGTACGATCCTTTGGCAACC | 66 | 377 |
| FluHA3-1 A/B 586/475 | 60 | HA3-L375 | GAAGTCTTCATTGATAAACTCCAG | 56 | 260 |
| FluHA3-2 A/B 586/475 | 60 | HA3-L380 | ATGCTGAGCCGACTCCAGTCC | 60 | 265 |
| FluHA5 A/B 646/395 | 62 | HA5human-L147 | AGGyTTCACTCCATTTAGATCGCA | 64 | 105 |

TABLE 12

| | | | | |
|---|---|---|---|---|
| | | | Previous | |
| Primer sequence | Name | Target | Masscode | Panel |

| | | | | | |
|---|---|---|---|---|---|
| TACAGCACGCTCAACACCAACGCCT | 25 | CMV-U421 | Citomegalovirus | | Respiratory |
| AACAGAAGGAAATGATGGTGGAAC | 24 | HPIV4A-U191 | HPIV4A | | Respiratory |
| AGAAGAAAACAACGATGAGACAAGG | 25 | HPIV4B-U194 | HPIV4B | | Respiratory |
| CAAGCATCATGATYGCCATTCCTGG | 25 | MEA-U1103 | Measles | | Respiratory |
| ACGTGGATCGTCGGATCAGTTGT | 23 | VZV-U138 | VZV | | Respiratory |
| TTCTTIGGAGCAGCIGGAAGCACIATGG | 28 | SK68i | HIV1 | | Respiratory |
| GGCTGCACGCCCTATGATA | 18 | HIV2TMFPR2 | HIV2 | | Respiratory |
| AGCGATAGCTTTCTCCAAGTGG | 22 | SPPLY-U532 | Streptococcus pneumonie | | Respiratory |
| AAGCTCCTTGMATTTTTTGTATTAGAA | 27 | HINF-U82 | Haemophilus influenza | | Respiratory |
| CCCGGATGCGGTCCAGACGATTAT | 24 | HSV-U27 | Herpes Simplex | | Respiratory |
| AAGTCCAAAGGCAGGRCTGTTATC | 24 | Mv-Can-U918 | Metaneumovirus Canadian | | Respiratory |
| CCCMTTYAACCACCACCG | 18 | ADV2F-A | Adenovirus | Adenovirus2 | Respiratory |
| | | | | 503 | |
| TCCTCCGGCCCCTGAATGCGGCTAATCC | 28 | 5UTR-U447 | Enterovirus | EnteroVirus | Respiratory |
| | | | | 702 | |
| TCCGGCCCCTGAATGCGGCTAATCC | 25 | 5UTR-U450 | Enterovirus | EnteroVirus | Respiratory |
| | | | | 702 | |
| CCCCTGAATGCGGCTAATCC | 20 | 5UTR-u457 | Enterovirus | EnteroVirus | Respiratory |
| | | | | 702 | |
| CCCGGCCTTCACCACCAACCGAAAA | 25 | CMV-L501 | Citomegalovirus | | Respiratory |
| TGCTGTGGATGTATGGGCAG | 20 | HPIV4A-L269 | HPIV4a | | Respiratory |
| GTTTCCCTGGTTCACTCTCTTCA | 23 | HPIV4B-L306 | HPIV4b | | Respiratory |
| CCTGAATCYCTGCCTATGATGGGTTT | 26 | MEA-L1183 | Measles | | Respiratory |
| TCGCTATGTGCTAAAACACGCGG | 23 | VZV-L196 | VZV | | Respiratory |
| TTMATGCCCCAGACIGTIAGTTICAACA | 28 | SK69i | HIV1 | | Respiratory |
| TCTGCATGGCTGCTTGATG | 18 | HIV2TMRPR2 | HIV2 | | Respiratory |
| CTTAGCCAACAAATCGTTTACCG | 23 | SPPLY-L606 | Streptococcus pneumonie | | Respiratory |
| GCTGAATTGGCTTRGATACCGAG | 23 | Hinf-L158 | Haemophilus influenza | | Respiratory |
| CCCGCGGAGGTTGTACAAAAAGCT | 24 | HSV-L121 | Herpes Simplex | | Respiratory |
| CCTGAAGCATTRCCAAGAACAACAC | 25 | MV-Can-L992 | Metaneumovirus Canadian | | Respiratory |
| ACATCCTTBCKGAAGTTCCA | 20 | ADV1R-A | Adenovirus | Adenovirus2 | Respiratory |
| | | | | 630 | |
| GAAACACGGWCACCCAAAGTASTCG | 25 | 5UTR-L541 | Enterovirus | EnteroVirus | Respiratory |
| | | | | 495 | |
| AACACCGGGTCTTAATTCTTATATCAA | 27 | EboZa-U234 | Ebola Zaire | | Hemorrhagic Fevers |
| TTCCGTCACAAGCCGAAATT | 20 | Mar-U292 | Marburg | | Hemorrhagic Fevers |
| AGAACACGTGCCGCTTACGCCCA | 23 | CCHV-U4 | CCHV | | Hemorrhagic Fevers |
| TCCCAAAGATGTTAGTGCCTGA | 22 | Sabia-U344 | Sabia | | Hemorrhagic Fevers |
| CCACCCGTCACCTGAGAGACACAATT | 28 | Machupo-U212 | Machupo | | Hemorrhagic Fevers |
| GCTGGGAGCGCGGTATC | 17 | YF-U186 | Yellow Fever | | Hemorrhagic Fevers |
| GGATTGACCTGTGCCTGTTGC | 21 | RVF-U578 | Rift Valley fever | | Hemorrhagic Fevers |
| TCTGAAGCCATTGGCCGT | 18 | Nmen-U829 | Neisseria meningitidis | | Hemorrhagic Fevers |
| CRTATTATTAMTGGCTATAAATGTTGC | 27 | RSF-U255 | Rickettsia Spotted fever | | Hemorrhagic Fevers |
| YACAATGACMGATGAGGTTGTRGC | 24 | Bburg-U896 | Borrelia burgdorferi | | Hemorrhagic Fevers |
| GATGGAGGRTGCATCATGG | 18 | OMSK-U171 | OMSK | | Hemorrhagic Fevers |
| AACTTAGGAGCTACCCAAAACAGC | 24 | CHKP-U68 | Chikungunya POL | | Hemorrhagic Fevers |
| CAATGTCYTMGCCTGGACACCT | 23 | CHKE-U223 | Chikungunya ENV | | Hemorrhagic Fevers |
| AYACAGCAGCAGTTAGCCTCCT | 22 | HAN-U179 | Hantaan | | Hemorrhagic Fevers |
| ATGAARGCAGATGARATYACACC | 23 | DOB-U222 | Dobrava | | Hemorrhagic Fevers |
| AAGGTGTTTTTGATCAGGCTAGAGA | 25 | TAC-U114 | Tacaribe | | Hemorrhagic Fevers |
| GCCRTGTGARTGCCTRCTTCCATT | 24 | GUAV-U321 | Guanarito | | Hemorrhagic Fevers |
| CAGGATTGCAGCAGGGAAGA | 20 | SEO-U243 | Seoul | | Hemorrhagic Fevers |
| TGGAAGCCTGGCTGAAAGAG | 20 | KYF-U170 | Kyasanur forest | | Hemorrhagic Fevers |
| TGACCTTYACMAATGAYTCCAT | 22 | LCMV-U47b | LCMV | | Hemorrhagic Fevers |
| GGTGGTAAAATTCCCATAGTAGTTCTTT | 28 | EboZA-L319 | Ebola Zaire | | Hemorrhagic Fevers |
| TTATTTTAGTTGAGAAAAGAGGTTCATGC | 29 | Mar-L372 | Marburg | | Hemorrhagic Fevers |
| CCATTCYTTYTTRAACTCYTCAAACCA | 27 | CCHV-L120 | CCHV | | Hemorrhagic Fevers |
| CCTGCACTGACAATCGCTTG | 20 | SABIA-L424 | Sabia | | Hemorrhagic Fevers |
| TGCAAGTCAAGCGAAAAGAGGGGATG | 26 | Machupo-L290 | Machupo | | Hemorrhagic Fevers |
| GGAAGCCCAATGGTCCTCAT | 20 | YF-L249 | Yellow Fever | | Hemorrhagic Fevers |
| GCATTAGAAATGTCCTCTTTTGCTGC | 26 | RVF-L660 | Rift Valley fever | | Hemorrhagic Fevers |
| CAAACACACCACGCGCAT | 18 | Nmen-L892 | Neisseria meningitidis | | Hemorrhagic Fevers |
| ACKRTTTAAAGTTAARCTTTTGCC | 24 | RSF-L394 | Rickettsia Spotted fever | | Hemorrhagic Fevers |
| GCAATGACAAAACATATTGRGGAASTTGA | 29 | Bburg-L977 | Borrelia burgdorferi | | Hemorrhagic Fevers |
| TGACCACTTGGCCTGATCC | 19 | OMSK-L234 | OMSK | | Hemorrhagic Fevers |
| GGACGGTACAGGCGCTTCTG | 19 | CHKP-L132 | Chikungunya POL | | Hemorrhagic Fevers |
| TCRCCAAATTGTCCTGGTCTTCCTG | 25 | CHKE-L310 | Chikungunya ENV | | Hemorrhagic Fevers |
| GCTGCCGTARGTAGTCCCTGTT | 22 | HAN-L245 | Hantaan | | Hemorrhagic Fevers |
| CCTGRGCTGGRTATARTCCACA | 22 | DOB-L289 | Dobrava | | Hemorrhagic Fevers |
| CCATCCTTGATGGTGGTAACATG | 23 | TAC-L192 | Tacaribe | | Hemorrhagic Fevers |
| TATGTRCACTGYTTCAGAAAACCTCA | 26 | GUA-L265 | Guanarito | | Hemorrhagic Fevers |
| ATGATCACCAGGYTCTACCCC | 21 | SEOUL-L309 | Seoul | | Hemorrhagic Fevers |
| TCATCCCCACTGACCAGCAT | 20 | KYF-L233 | Kyassanur forest | | Hemorrhagic Fevers |
| TATRCTCATGAGTGTGTGGTCAA | 23 | LCMV-L142a | LCMV | Same than | Hemorrhagic Fevers |
| | | | | below | |
| TATRCTCATAAGTGTGTGATCAA | 23 | LCMV-L142b | LCMV | Same than | Hemorrhagic Fevers |
| | 1598 | | | above | |

### Example 7

Efficient laboratory diagnosis of infectious diseases is increasingly important to clinical management and public health. Methods to directly detect nucleic acids of microbial pathogens in clinical specimens are rapid, sensitive, and may succeed when culturing the organism fails. Clinical syndromes are infrequently specific for single pathogens; thus, assays are needed that allow multiple agents to be simultaneously considered. Current multiplex assays employ gel-based formats in which products are distinguished by size, fluorescent reporter dyes that vary in color, or secondary enzyme hybridization assays. Gel-based assays are reported that detect 2-8 different targets with sensitivities of 2-100 PFU or less than 1-5 PFU, depending on whether amplification is carried out in a single or nested format, respectively (1-4). Fluorescence reporter systems achieve quantitative detection with sensitivity similar to that of nested amplification; however, their capacity to simultaneously query multiple targets is limited to the number of fluorescent emission peaks that can be unequivocally resolved. At present, up to 4 fluorescent reporter dyes can be detected simultaneously (5,6). Multiplex detection of up to 9 pathogens has been achieved in hybridization enzyme systems; however, the method requires cumbersome postamplification processing (7).

Experimental Results

To address the need for sensitive multiplex assays in diagnostic molecular microbiology, we created a polymerase chain reaction (PCR) platform in which microbial gene targets are coded by a library of 64 distinct Masscode tags (Qiagen Masscode technology, Qiagen, Hilden, Germany). A schematic representation of this approach is shown in FIG. 22. Microbial nucleic acids (RNA, DNA, or both) are amplified by multiplex reverse transcription (RT)-PCR using primers labeled by a photocleavable link to molecular tags of different molecular weight. After removing unincorporated primers, tags are released by UV irradiation and analyzed by mass spectrometry. The identity of the microbe in the clinical sample is determined by its cognate tags. As a first test of this technology, we focused on respiratory disease because differential diagnosis is a common clinical challenge, with implications for outbreak control and individual case management. Multiplex primer sets were designed to identify up to 22 respiratory pathogens in a single Mass Tag PCR reaction; sensitivity was established by using synthetic DNA and RNA standards as well as titered viral stocks; the utility of Mass Tag PCR was determined in blinded analysis of previously diagnosed clinical specimens. Oligonucleotide primers were designed in conserved genomic regions to detect the broadest number of members for a given pathogen species by efficiently amplifying a 50- to 300-bp product. In some instances, we selected established primer sets; in others, we used a software program designed to cull sequence information from GenBank, perform multiple alignments, and maximize multiplex performance by selecting primers with uniform melting temperatures and minimal cross-hybridization potential (Appendix Table, available at http://www.cdc. gov/ncidod/eid/vol11no02/04-0492_app.htm). Primers, synthesized with a 5′C6 spacer and aminohexyl modification, were covalently conjugated by a photocleavable link to Masscode tags (Qiagen Masscode technology) (8,9). Masscode tags have a modular structure, including a tetrafluorophenyl ester for tag conjugation to primary amines; an o-nitrobenzyl photolabile linker for photoredox cleavage of the tag from the analyte; a mass spectrometry sensitivity enhancer, which improves the efficiency of atmospheric pressure chemical ionization of the cleaved tag; and a variable mass unit for variation of the cleaved tag mass (8,10-12). A library of 64 different tags has been established. Forward and reverse primers in individual primer sets are labeled with distinct molecular weight tags. Thus, amplification of a microbial gene target produces a dual signal that allows assessment of specificity. Gene target standards were cloned by PCR into pCR2.1-TOPO (Invitrogen, Carlsbad, Calif., USA) by using DNA template (bacterial and DNA viral targets) or cDNA template (RNA viral targets) obtained by reverse transcription of extracts from infected cultured cells or by assembly of overlapping synthetic polynucleotides. Assays were initially established by using plasmid standards diluted in 2.5-μg/mL human placenta DNA (Sigma, St. Louis, Mo., USA) and subjected to PCR amplification with a multiplex PCR kit (Qiagen), primers at 0.5 μmol/L each, and the following cycling protocol: an annealing step with a temperature reduction in 1° C. increments from 65° C. to 51° C. during the first 15 cycles and then continuing with a cycling profile of 94° C. for 20 s, 50° C. for 20 s, and 72° C. for 30 s in an MJ PTC200 thermal cycler (MJ Research, Waltham, Mass., USA). Amplification products were separated from unused primers by using QIAquick 96 PCR purification cartridges (Qiagen, with modified binding and wash buffers). Masscode tags were decoupled from amplified products through UV light-induced photolysis in a flow cell and analyzed in a single quadrapole mass spectrometer using positive-mode atmospheric pressure chemical ionization (Agilent Technologies, Palo Alto, Calif., USA). A detection threshold of 100 DNA copies was determined for 19 of 22 cloned targets by using a 22-plex assay (Table 1). Many respiratory pathogens have RNA genomes; thus, where indicated, assay sensitivity was determined by using synthetic RNA standards or RNA extracts of viral stocks. Synthetic RNA standards were generated by using T7 polymerase and linearized plasmid DNA. After quantitation by UV spectrometry, RNA was serially diluted in 2.5-μg/mL yeast tRNA (Sigma), reverse transcribed with random hexamers by using Superscript II (Invitrogen, Carlsbad, Calif., USA), and used as template for Mass Tag PCR. As anticipated, sensitivity was reduced by the use of RNA instead of DNA templates (Table 15).

TABLE 15

| | Detection threshold |
|---|---|
| Pathogen or protein | (DNA copies/RNA copies) |
| Influenza A matrix | 100/1,000 |
| Influenza A N1 | 100/NA |
| Influenza A N2 | 100/NA |
| Influenza A H1 | 100/NA |
| Influenza A H2 | 100/NA |
| Influenza A H3 | 100/NA |
| Influenza A H5 | 100/NA |
| Influenza B H | 500/1,000 |
| RSV group A | 100/1,000 |
| RSV group B | 100/500 |
| Metapneumovirus | 100/1,000 |
| CoV-SARS | 100/500 |
| CoV-OC43 | 100/500 |
| CoV-229E | 100/500 |
| HPIV-1 | 100/1,000 |
| HPIV-2 | 100/1,000 |
| HPIV-3 | 100/500 |
| Chlamydia pneumoniae | 100/NA |
| Mycoplasma pneumoniae | 100/NA |
| Legionella pneumophila | 100/NA |
| Enterovirus (genus) | 500/1,000 |
| Adenovirus (genus) | 5,000/NA |

*NA, not assessed;
RSV, respiratory syncytial virus;
CoV, coronavirus;
SARS, severe acute respiratory syndrome;
HPIV, human parainfluenza virus.

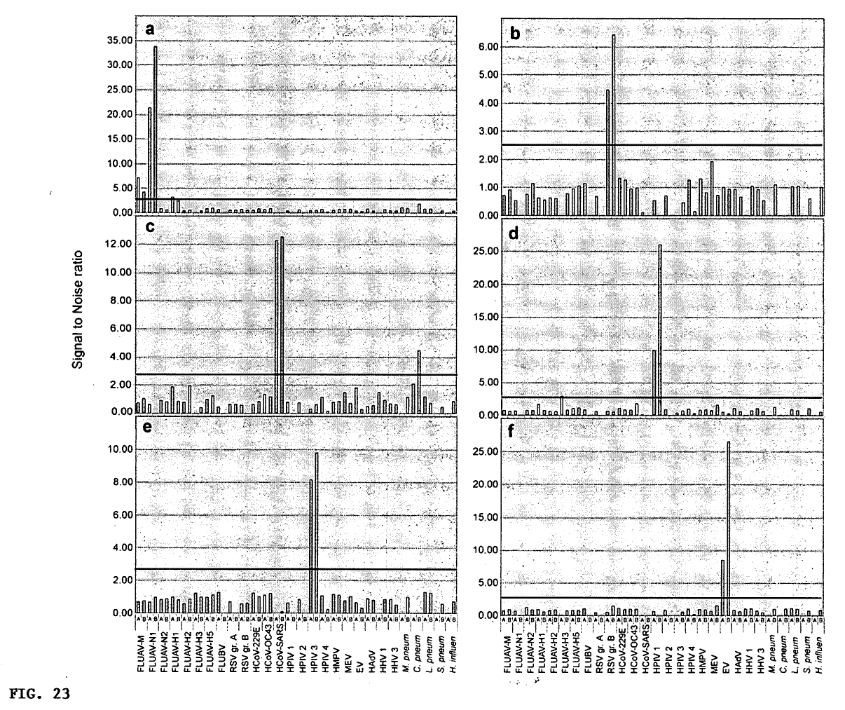

The sensitivity of Mass Tag PCR to detect live virus was tested by using RNA extracted from serial dilutions of titered stocks of coronaviruses (severe acute respiratory syndrome [SARS] and OC43) and parainfluenzaviruses (HPIV 2 and 3). A 100-μL volume of each dilution was analyzed. RNA extracted from a 1-TCID50/mL dilution, representing 0.025 TCID50 per PCR reaction, was consistently positive in Mass Tag PCR. RNA extracted from banked sputum, nasal swabs, and pulmonary washes of persons with respiratory infection was tested by using an assay panel comprising 30 gene targets that represented 22 respiratory pathogens. Infection in each of these persons had been previously diagnosed through virus isolation, conventional nested RT-PCR, or both. Reverse transcription was performed using random hexamers, and Mass Tag PCR results were consistent in all cases with the established diagnosis. Infections with respiratory syncytial virus, human parainfluenza virus, SARS coronavirus, adenovirus, enterovirus, metapneumovirus, and influenza virus were correctly identified (Table 16 and FIG. 23).

TABLE 16

| Pathogen | No. positive/no. tested† |
|---|---|
| RSV A | 2/2 |
| RSV B | 3/3 |
| HPIV-1 | 1/1 |
| HPIV-3 | 2/2 |
| HPIV-4 | 2/2 |
| CoV-SARS | 4/4 |
| Metapneumovirus | 2/3 |
| Influenza B | 1/3 |
| Influenza A | 2/6 |
| Adenovirus | 2/2 |
| Enterovirus | 2/2 |

*RSV, respiratory syncytial virus;
HPIV, human parainfluenza virus;
CoV, coronavirus;
SARS, severe acute respiratory syndrome.
†No. positive and consistent with previous diagnosis/number tested (with respective previous diagnosis).

A panel comprising gene targets representing 17 pathogens related to central nervous system infectious disease (influenza A virus matrix gene; influenza B virus; human coronaviruses 229E, OC43, and SARS; enterovirus; adenovirus; human herpesvirus-1 and -3; West Nile virus; St. Louis encephalitis virus; measles virus; HIV-1 and -2; and Streptococcus pneumoniae, Haemophilus influenzae, and Nesseria meningitidis) was applied to RNA obtained from banked samples of cerebrospinal fluid and brain tissue that had been previously characterized by conventional diagnostic RT-PCR. Two of 3 cases of West Nile virus encephalitis were correctly identified. Eleven of 12 cases of enteroviral meningitis were detected representing serotypes CV-B2, CV-B3, CV-B5, E-6, E-11, E-13, E-18, and E-30 (data not shown).

### CONCLUSIONS

Our results indicate that Mass Tag PCR is a sensitive and specific tool for molecular characterization of microflora. The advantage of Mass Tag PCR is its capacity for multiplex analysis. Although the use of degenerate primers (e.g., enteroviruses and adenoviruses, and Table 16) may reduce sensitivity, the limit of multiplexing to detect specific targets will likely be defined by the maximal primer concentration that can be accommodated in a PCR mix. Analysis requires the purification of product from unincorporated primers and mass spectroscopy. Although these steps are now performed manually, and mass spectrometers are not yet widely distributed in clinical laboratories, the increasing popularity of mass spectrometry in biomedical sciences and the advent of smaller, lower-cost instruments could facilitate wider use additional pathogen panels, our continuing work is focused on optimizing multiplexing, sensitivity, and throughput. Potential applications include differential diagnosis of infectious diseases, blood product surveillance, forensic microbiology, and biodefense.

1. A method for simultaneously detecting in a sample the presence of one or more of a plurality of different target nucleic acids comprising the steps of:
(a) contacting the sample with a plurality of nucleic acid primers simultaneously and under conditions permitting, and for a time sufficient for, primer extension to occur, wherein (i) for each target nucleic acid at least one predetermined primer is used which is specific for that target nucleic acid, (ii) each primer has a mass tag of predetermined size bound thereto via a labile bond, and (iii) the mass tag bound to any primer specific for one target nucleic acid has a different mass than the mass tag bound to any primer specific for any other target nucleic acid;
(b) separating any unextended primers from any extended primers;
(c) simultaneously cleaving the mass tags from any extended primers; and
(d) simultaneously determining the presence and sizes of any mass tags so cleaved,
wherein the presence of a cleaved mass tag having the same size as a mass tag of predetermined size previously bound to a predetermined primer indicates the presence in the sample of the target nucleic acid specifically recognized by that predetermined primer.

2. The method of claim 1, wherein the method detects the presence in the sample of 10 or more different target nucleic acids.

3. The method of claim 1, wherein the method detects the presence in the sample of 50 or more different target nucleic acids.

4. The method of claim 1, wherein the method detects the presence in the sample of 100 or more different target nucleic acids.

5. The method of claim 1, wherein the method detects the presence in the sample of 200 or more different target nucleic acids.

6. The method of claim 1, wherein the sample is contacted with 4 or more different primers.

7. The method of claim 1, wherein the sample is contacted with 10 or more different primers.

8. The method of claim 1, wherein the sample is contacted with 50 or more different primers.

9. The method of claim 1, wherein the sample is contacted with 100 or more different primers.

10. The method of claim 1, wherein the sample is contacted with 200 or more different primers.

11. The method of claim 1, wherein one or more primers comprises the sequence set forth in one of SEQ ID NOs:1-96.

12. The method of claim 1, wherein at least two different primers are specific for the same target nucleic acid.

13. The method of claim 12, wherein a first primer is a forward primer for the target nucleic acid and a second primer is a reverse primer for the same target nucleic acid.

14. The method of claim 13, wherein the mass tags bound to the first and second primers are of the same size.

15. The method of claim 13, wherein the mass tags bound to the first and second primers are of a different size.

16. The method of claim 12, wherein a first primer is directed to a 5′-UTR of the target nucleic acid and a second primer is directed to a 3D polymerase region of the target nucleic acid.

17. The method of claim 1, wherein each primer is from 15 to 30 nucleotides in length.

18. The method of claim 1, wherein each mass tag has a molecular weight of from 100 Da to 2,500 Da.

19. The method of claim 1, wherein the labile bond is a photolabile bond.

20. The method of claim 19, wherein the photolabile bond is cleavable by ultraviolet light.

21. The method of claim 1, wherein at least one target nucleic acid is from a pathogen.

22. The method of claim 21, wherein the pathogen is selected from the group consisting of B. anthracis, a Dengue virus, a West Nile virus, Japanese encephalitis virus, St. Louis encephalitis virus, Yellow Fever virus, La Crosse virus, California encephalitis virus, Rift Valley Fever virus, CCHF virus, VEE virus, EEE virus, WEE virus, Ebola virus, Marburg virus, LCMV, Junin virus, Machupo virus, Variola virus, SARS corona virus, an enterovirus, an influenza virus, a parainfluenza virus, a respiratory syncytial virus, a bunyavirus, a flavivirus, and an alphavirus.

23. The method of claim 21, wherein the pathogen is a respiratory pathogen.

24. The method of claim 23, wherein the respiratory pathogen is selected from the group consisting of respiratory syncytial virus A, respiratory syncytial virus B, Influenza A (N1), Influenza A (N2), Influenza A (M), Influenza A (H1), Influenza A (H2), Influenza A (H3), Influenza A (H5), Influenza B, SARS coronavirus, 229E coronavirus, OC43 coronavirus, Metapneumovirus European, Metapneumovirus Canadian, Parainfluenza 1, Parainfluenza 2, Parainfluenza 3, Parainfluenza 4A, Parainfluenza 4B, Cytomegalovirus, Measles virus, Adenovirus, Enterovirus, M. pneumoniae, L. pneumophilae, and C. pneumoniae.

25. The method of claim 21, wherein the pathogen is an encephalitis-inducing pathogen.

26. The method of claim 25, wherein the encephalitis-inducing pathogen is selected from the group consisting of West Nile virus, St. Louis encephalitis virus, Herpes Simplex virus, HIV 1, HIV 2, N. meningitides, S. pneumoniae, H. influenzae, Influenza B, SARS coronavirus, 229E-CoV, OC43-CoV, Cytomegalovirus, and a Varicella Zoster virus.

27. The method of claim 21, wherein the pathogen is a hemorrhagic fever-inducing pathogen.

28. The method of claim 1, wherein the sample is a forensic sample.

29. The method of claim 1, wherein the sample is a food sample.

30. The method of claim 1, wherein the sample is blood, or a derivative of blood.

31. The method of claim 1, wherein the sample is a biological warfare agent or a suspected biological warfare agent.

32. The method of claim 1, wherein the mass tag is selected from the group consisting of:

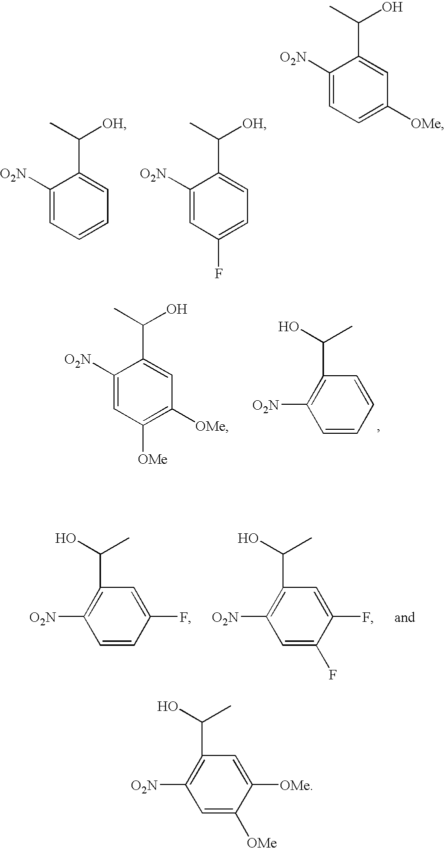

33. The method of claim 1, wherein the presence and size of any cleaved mass tag is determined by mass spectrometry.

34. The method of claim 33, wherein the mass spectrometry is selected from the group consisting of atmospheric pressure chemical ionization mass spectrometry, electrospray ionization mass spectrometry, and matrix assisted laser desorption ionization mass spectrometry.

35. The method of claim 1, wherein the target nucleic acid is a ribonucleic acid.

36. The method of claim 1, wherein the target nucleic acid is a deoxyribonucleic acid.

37. The method of claim 1, wherein the target nucleic acid is from a viral source.

38-41. (canceled)
